SEQUENCE LISTING (123 sequences)

SEQ ID NO: 1 - DNA, 1736 bp, Artificial Sequence
  misc_binding
acaccgtgcgataacactcgatcaatcttcccgtctcaccggtctccctgggtgacagagcaagaccctgtacaccgtgcgataacactcgatcaatcttcccgtctcaccggtctcccgatccaagttgacttggctgagatgtacaccgtgcgataacactcgatcaatcttcccgtctcaccggtctcccgcgtttgtgtgtgcatctgtaagcatgtacaccgtgcgataacactcgatcaatcttcccgtctcaccggtctcccgtgcacacttggacagcatttcctgtacaccgtgcgataacactcgatcaatcttcccgtctcaccggtctcccgaggaagggctgtgtttcagggctgtacaccgtgcgataacactcgatcaatcttcccgtctcaccggtctccccgcttttctggccagaaacctctgtacaccgtgcgataacactcgatcaatcttcccgtctcaccggtctcccccagcttccctgattcttcagcttgtacaccgtgcgataacactcgatcaatcttcccgtctcaccggtctcccaaaattaacttctctggtgtgtggagatgtacaccgtgcgataacactcgatcaatcttcccgtctcaccggtctccccctaacctatcatccatccttatctcttgtacaccgtgcgataacactcgatcaatcttcccgtctcaccggtctccctgcacccaacattctaacaaaaggctgtacaccgtgcgataacactcgatcaatcttcccgtctcaccggtctccccaataggtttttaaggaacaggtggtgtacaccgtgcgataacactcgatcaatcttcccgtctcaccggtctcccgggtgattcccattggcctgtacaccgtgcgataacactcgatcaatcttcccgtctcaccggtctcccgaagtagctgctgagtgatttgtctgtacaccgtgcgataacactcgatcaatcttcccgtctcaccggtctccccctctccaccctatagaccctgtacaccgtgcgataacactcgatcaatcttcccgtctcaccggtctcccctaattaaagtggtgtcccagataatctgtacaccgtgcgataacactcgatcaatcttcccgtctcaccggtctcccctctgacccatctaacgcctatctgtacaccgtgcgataacactcgatcaatcttcccgtctcaccggtctccctgagtagctgggactacaggcatgtacaccgtgcgataacactcgatcaatcttcccgtctcaccggtctccctgacgcggtctccgcggtgtacaccgtgcgataacactcgatcaatcttcccgtctcaccggtctcccctttttattaaatgctttccatgtatcaagttctgtacaccgtgcgataacactcgatcaatcttcccgtctcaccggtctcccctttcccaattctccttcagtcctgtacaccgtgcgataacactcgatcaatcttcccgtctcaccggtctcccgatatcagggaagatgaaaaaagagactgtacaccgtgcgataacactcgatcaatcttcccgtctcaccggtctcccgtcccagaggcccttgtcagtgtacaccgtgcgataacactcgatcaatcttcccgtctcaccggtctccccctgggctctgtaaagaatagtgtacacgaaccgattggtgcagtcttc

SEQ ID NO: 2 - DNA, 1736 bp, Artificial Sequence
  misc_binding
acaccgtgcgataacactcgatcaatctgagggatatgcatcaccttttgggtgacagagcaagaccctgtacaccgtgcgataacactcgatcaatctgagggatatgcatcacctttgatccaagttgacttggctgagatgtacaccgtgcgataacactcgatcaatctgagggatatgcatcacctttgcgtttgtgtgtgcatctgtaagcatgtacaccgtgcgataacactcgatcaatctgagggatatgcatcacctttgtgcacacttggacagcatttcctgtacaccgtgcgataacactcgatcaatctgagggatatgcatcacctttgaggaagggctgtgtttcagggctgtacaccgtgcgataacactcgatcaatctgagggatatgcatcacctttcgcttttctggccagaaacctctgtacaccgtgcgataacactcgatcaatctgagggatatgcatcacctttccagcttccctgattcttcagcttgtacaccgtgcgataacactcgatcaatctgagggatatgcatcacctttaaaattaacttctctggtgtgtggagatgtacaccgtgcgataacactcgatcaatctgagggatatgcatcacctttcctaacctatcatccatccttatctcttgtacaccgtgcgataacactcgatcaatctgagggatatgcatcaccttttgcacccaacattctaacaaaaggctgtacaccgtgcgataacactcgatcaatctgagggatatgcatcacctttcaataggtttttaaggaacaggtggtgtacaccgtgcgataacactcgatcaatctgagggatatgcatcacctttgggtgattcccattggcctgtacaccgtgcgataacactcgatcaatctgagggatatgcatcacctttgaagtagctgctgagtgatttgtctgtacaccgtgcgataacactcgatcaatctgagggatatgcatcacctttcctctccaccctatagaccctgtacaccgtgcgataacactcgatcaatctgagggatatgcatcacctttctaattaaagtggtgtcccagataatctgtacaccgtgcgataacactcgatcaatctgagggatatgcatcacctttctctgacccatctaacgcctatctgtacaccgtgcgataacactcgatcaatctgagggatatgcatcaccttttgagtagctgggactacaggcatgtacaccgtgcgataacactcgatcaatctgagggatatgcatcaccttttgacgcggtctccgcggtgtacaccgtgcgataacactcgatcaatctgagggatatgcatcacctttctttttattaaatgctttccatgtatcaagttctgtacaccgtgcgataacactcgatcaatctgagggatatgcatcacctttctttcccaattctccttcagtcctgtacaccgtgcgataacactcgatcaatctgagggatatgcatcacctttgatatcagggaagatgaaaaaagagactgtacaccgtgcgataacactcgatcaatctgagggatatgcatcacctttgtcccagaggcccttgtcagtgtacaccgtgcgataacactcgatcaatctgagggatatgcatcacctttcctgggctctgtaaagaatagtgtacacgaaccgattggtgcagtcttc

SEQ ID NO: 3 - DNA, 1736 bp, Artificial Sequence
  misc_binding
acaccgtgcgataacactcgatcaatctagcacacgtaatcgtttccgtgggtgacagagcaagaccctgtacaccgtgcgataacactcgatcaatctagcacacgtaatcgtttccggatccaagttgacttggctgagatgtacaccgtgcgataacactcgatcaatctagcacacgtaatcgtttccggcgtttgtgtgtgcatctgtaagcatgtacaccgtgcgataacactcgatcaatctagcacacgtaatcgtttccggtgcacacttggacagcatttcctgtacaccgtgcgataacactcgatcaatctagcacacgtaatcgtttccggaggaagggctgtgtttcagggctgtacaccgtgcgataacactcgatcaatctagcacacgtaatcgtttccgcgcttttctggccagaaacctctgtacaccgtgcgataacactcgatcaatctagcacacgtaatcgtttccgccagcttccctgattcttcagcttgtacaccgtgcgataacactcgatcaatctagcacacgtaatcgtttccgaaaattaacttctctggtgtgtggagatgtacaccgtgcgataacactcgatcaatctagcacacgtaatcgtttccgcctaacctatcatccatccttatctcttgtacaccgtgcgataacactcgatcaatctagcacacgtaatcgtttccgtgcacccaacattctaacaaaaggctgtacaccgtgcgataacactcgatcaatctagcacacgtaatcgtttccgcaataggtttttaaggaacaggtggtgtacaccgtgcgataacactcgatcaatctagcacacgtaatcgtttccggggtgattcccattggcctgtacaccgtgcgataacactcgatcaatctagcacacgtaatcgtttccggaagtagctgctgagtgatttgtctgtacaccgtgcgataacactcgatcaatctagcacacgtaatcgtttccgcctctccaccctatagaccctgtacaccgtgcgataacactcgatcaatctagcacacgtaatcgtttccgctaattaaagtggtgtcccagataatctgtacaccgtgcgataacactcgatcaatctagcacacgtaatcgtttccgctctgacccatctaacgcctatctgtacaccgtgcgataacactcgatcaatctagcacacgtaatcgtttccgtgagtagctgggactacaggcatgtacaccgtgcgataacactcgatcaatctagcacacgtaatcgtttccgtgacgcggtctccgcggtgtacaccgtgcgataacactcgatcaatctagcacacgtaatcgtttccgctttttattaaatgctttccatgtatcaagttctgtacaccgtgcgataacactcgatcaatctagcacacgtaatcgtttccgctttcccaattctccttcagtcctgtacaccgtgcgataacactcgatcaatctagcacacgtaatcgtttccggatatcagggaagatgaaaaaagagactgtacaccgtgcgataacactcgatcaatctagcacacgtaatcgtttccggtcccagaggcccttgtcagtgtacaccgtgcgataacactcgatcaatctagcacacgtaatcgtttccgcctgggctctgtaaagaatagtgtacacgaaccgattggtgcagtcttc

SEQ ID NO: 4 - DNA, 1736 bp, Artificial Sequence
  misc_binding
acaccgtgcgataacactcgatcaatctatgtgggtgaggtccgaccctgggtgacagagcaagaccctgtacaccgtgcgataacactcgatcaatctatgtgggtgaggtccgacccgatccaagttgacttggctgagatgtacaccgtgcgataacactcgatcaatctatgtgggtgaggtccgacccgcgtttgtgtgtgcatctgtaagcatgtacaccgtgcgataacactcgatcaatctatgtgggtgaggtccgacccgtgcacacttggacagcatttcctgtacaccgtgcgataacactcgatcaatctatgtgggtgaggtccgacccgaggaagggctgtgtttcagggctgtacaccgtgcgataacactcgatcaatctatgtgggtgaggtccgaccccgcttttctggccagaaacctctgtacaccgtgcgataacactcgatcaatctatgtgggtgaggtccgacccccagcttccctgattcttcagcttgtacaccgtgcgataacactcgatcaatctatgtgggtgaggtccgacccaaaattaacttctctggtgtgtggagatgtacaccgtgcgataacactcgatcaatctatgtgggtgaggtccgaccccctaacctatcatccatccttatctcttgtacaccgtgcgataacactcgatcaatctatgtgggtgaggtccgaccctgcacccaacattctaacaaaaggctgtacaccgtgcgataacactcgatcaatctatgtgggtgaggtccgaccccaataggtttttaaggaacaggtggtgtacaccgtgcgataacactcgatcaatctatgtgggtgaggtccgacccgggtgattcccattggcctgtacaccgtgcgataacactcgatcaatctatgtgggtgaggtccgacccgaagtagctgctgagtgatttgtctgtacaccgtgcgataacactcgatcaatctatgtgggtgaggtccgaccccctctccaccctatagaccctgtacaccgtgcgataacactcgatcaatctatgtgggtgaggtccgacccctaattaaagtggtgtcccagataatctgtacaccgtgcgataacactcgatcaatctatgtgggtgaggtccgacccctctgacccatctaacgcctatctgtacaccgtgcgataacactcgatcaatctatgtgggtgaggtccgaccctgagtagctgggactacaggcatgtacaccgtgcgataacactcgatcaatctatgtgggtgaggtccgaccctgacgcggtctccgcggtgtacaccgtgcgataacactcgatcaatctatgtgggtgaggtccgacccctttttattaaatgctttccatgtatcaagttctgtacaccgtgcgataacactcgatcaatctatgtgggtgaggtccgacccctttcccaattctccttcagtcctgtacaccgtgcgataacactcgatcaatctatgtgggtgaggtccgacccgatatcagggaagatgaaaaaagagactgtacaccgtgcgataacactcgatcaatctatgtgggtgaggtccgacccgtcccagaggcccttgtcagtgtacaccgtgcgataacactcgatcaatctatgtgggtgaggtccgaccccctgggctctgtaaagaatagtgtacacgaaccgattggtgcagtcttc

SEQ ID NO: 5 - DNA, 1736 bp, Artificial Sequence
  misc_binding
acaccgtgcgataacactcgatcaatctaatattccatgttattcgtctgggtgacagagcaagaccctgtacaccgtgcgataacactcgatcaatctaatattccatgttattcgtcgatccaagttgacttggctgagatgtacaccgtgcgataacactcgatcaatctaatattccatgttattcgtcgcgtttgtgtgtgcatctgtaagcatgtacaccgtgcgataacactcgatcaatctaatattccatgttattcgtcgtgcacacttggacagcatttcctgtacaccgtgcgataacactcgatcaatctaatattccatgttattcgtcgaggaagggctgtgtttcagggctgtacaccgtgcgataacactcgatcaatctaatattccatgttattcgtccgcttttctggccagaaacctctgtacaccgtgcgataacactcgatcaatctaatattccatgttattcgtcccagcttccctgattcttcagcttgtacaccgtgcgataacactcgatcaatctaatattccatgttattcgtcaaaattaacttctctggtgtgtggagatgtacaccgtgcgataacactcgatcaatctaatattccatgttattcgtccctaacctatcatccatccttatctcttgtacaccgtgcgataacactcgatcaatctaatattccatgttattcgtctgcacccaacattctaacaaaaggctgtacaccgtgcgataacactcgatcaatctaatattccatgttattcgtccaataggtttttaaggaacaggtggtgtacaccgtgcgataacactcgatcaatctaatattccatgttattcgtcgggtgattcccattggcctgtacaccgtgcgataacactcgatcaatctaatattccatgttattcgtcgaagtagctgctgagtgatttgtctgtacaccgtgcgataacactcgatcaatctaatattccatgttattcgtccctctccaccctatagaccctgtacaccgtgcgataacactcgatcaatctaatattccatgttattcgtcctaattaaagtggtgtcccagataatctgtacaccgtgcgataacactcgatcaatctaatattccatgttattcgtcctctgacccatctaacgcctatctgtacaccgtgcgataacactcgatcaatctaatattccatgttattcgtctgagtagctgggactacaggcatgtacaccgtgcgataacactcgatcaatctaatattccatgttattcgtctgacgcggtctccgcggtgtacaccgtgcgataacactcgatcaatctaatattccatgttattcgtcctttttattaaatgctttccatgtatcaagttctgtacaccgtgcgataacactcgatcaatctaatattccatgttattcgtcctttcccaattctccttcagtcctgtacaccgtgcgataacactcgatcaatctaatattccatgttattcgtcgatatcagggaagatgaaaaaagagactgtacaccgtgcgataacactcgatcaatctaatattccatgttattcgtcgtcccagaggcccttgtcagtgtacaccgtgcgataacactcgatcaatctaatattccatgttattcgtccctgggctctgtaaagaatagtgtacacgaaccgattggtgcagtcttc

SEQ ID NO: 6 - DNA, 1736 bp, Artificial Sequence
  misc_binding
acaccgtgcgataacactcgatcaatcttttgtagaaatacggggccttgggtgacagagcaagaccctgtacaccgtgcgataacactcgatcaatcttttgtagaaatacggggcctgatccaagttgacttggctgagatgtacaccgtgcgataacactcgatcaatcttttgtagaaatacggggcctgcgtttgtgtgtgcatctgtaagcatgtacaccgtgcgataacactcgatcaatcttttgtagaaatacggggcctgtgcacacttggacagcatttcctgtacaccgtgcgataacactcgatcaatcttttgtagaaatacggggcctgaggaagggctgtgtttcagggctgtacaccgtgcgataacactcgatcaatcttttgtagaaatacggggcctcgcttttctggccagaaacctctgtacaccgtgcgataacactcgatcaatcttttgtagaaatacggggcctccagcttccctgattcttcagcttgtacaccgtgcgataacactcgatcaatcttttgtagaaatacggggcctaaaattaacttctctggtgtgtggagatgtacaccgtgcgataacactcgatcaatcttttgtagaaatacggggcctcctaacctatcatccatccttatctcttgtacaccgtgcgataacactcgatcaatcttttgtagaaatacggggccttgcacccaacattctaacaaaaggctgtacaccgtgcgataacactcgatcaatcttttgtagaaatacggggcctcaataggtttttaaggaacaggtggtgtacaccgtgcgataacactcgatcaatcttttgtagaaatacggggcctgggtgattcccattggcctgtacaccgtgcgataacactcgatcaatcttttgtagaaatacggggcctgaagtagctgctgagtgatttgtctgtacaccgtgcgataacactcgatcaatcttttgtagaaatacggggcctcctctccaccctatagaccctgtacaccgtgcgataacactcgatcaatcttttgtagaaatacggggcctctaattaaagtggtgtcccagataatctgtacaccgtgcgataacactcgatcaatcttttgtagaaatacggggcctctctgacccatctaacgcctatctgtacaccgtgcgataacactcgatcaatcttttgtagaaatacggggccttgagtagctgggactacaggcatgtacaccgtgcgataacactcgatcaatcttttgtagaaatacggggccttgacgcggtctccgcggtgtacaccgtgcgataacactcgatcaatcttttgtagaaatacggggcctctttttattaaatgctttccatgtatcaagttctgtacaccgtgcgataacactcgatcaatcttttgtagaaatacggggcctctttcccaattctccttcagtcctgtacaccgtgcgataacactcgatcaatcttttgtagaaatacggggcctgatatcagggaagatgaaaaaagagactgtacaccgtgcgataacactcgatcaatcttttgtagaaatacggggcctgtcccagaggcccttgtcagtgtacaccgtgcgataacactcgatcaatcttttgtagaaatacggggcctcctgggctctgtaaagaatagtgtacacgaaccgattggtgcagtcttc

SEQ ID NO: 7 - DNA, 1736 bp, Artificial Sequence
  misc_binding
acaccgtgcgataacactcgatcaatctcgagcacaaccctaagtttatgggtgacagagcaagaccctgtacaccgtgcgataacactcgatcaatctcgagcacaaccctaagtttagatccaagttgacttggctgagatgtacaccgtgcgataacactcgatcaatctcgagcacaaccctaagtttagcgtttgtgtgtgcatctgtaagcatgtacaccgtgcgataacactcgatcaatctcgagcacaaccctaagtttagtgcacacttggacagcatttcctgtacaccgtgcgataacactcgatcaatctcgagcacaaccctaagtttagaggaagggctgtgtttcagggctgtacaccgtgcgataacactcgatcaatctcgagcacaaccctaagtttacgcttttctggccagaaacctctgtacaccgtgcgataacactcgatcaatctcgagcacaaccctaagtttaccagcttccctgattcttcagcttgtacaccgtgcgataacactcgatcaatctcgagcacaaccctaagtttaaaaattaacttctctggtgtgtggagatgtacaccgtgcgataacactcgatcaatctcgagcacaaccctaagtttacctaacctatcatccatccttatctcttgtacaccgtgcgataacactcgatcaatctcgagcacaaccctaagtttatgcacccaacattctaacaaaaggctgtacaccgtgcgataacactcgatcaatctcgagcacaaccctaagtttacaataggtttttaaggaacaggtggtgtacaccgtgcgataacactcgatcaatctcgagcacaaccctaagtttagggtgattcccattggcctgtacaccgtgcgataacactcgatcaatctcgagcacaaccctaagtttagaagtagctgctgagtgatttgtctgtacaccgtgcgataacactcgatcaatctcgagcacaaccctaagtttacctctccaccctatagaccctgtacaccgtgcgataacactcgatcaatctcgagcacaaccctaagtttactaattaaagtggtgtcccagataatctgtacaccgtgcgataacactcgatcaatctcgagcacaaccctaagtttactctgacccatctaacgcctatctgtacaccgtgcgataacactcgatcaatctcgagcacaaccctaagtttatgagtagctgggactacaggcatgtacaccgtgcgataacactcgatcaatctcgagcacaaccctaagtttatgacgcggtctccgcggtgtacaccgtgcgataacactcgatcaatctcgagcacaaccctaagtttactttttattaaatgctttccatgtatcaagttctgtacaccgtgcgataacactcgatcaatctcgagcacaaccctaagtttactttcccaattctccttcagtcctgtacaccgtgcgataacactcgatcaatctcgagcacaaccctaagtttagatatcagggaagatgaaaaaagagactgtacaccgtgcgataacactcgatcaatctcgagcacaaccctaagtttagtcccagaggcccttgtcagtgtacaccgtgcgataacactcgatcaatctcgagcacaaccctaagtttacctgggctctgtaaagaatagtgtacacgaaccgattggtgcagtcttc

SEQ ID NO: 8 - DNA, 1736 bp, Artificial Sequence
  misc_binding
acaccgtgcgataacactcgatcaatctaccgccggtttatggtggtgtgggtgacagagcaagaccctgtacaccgtgcgataacactcgatcaatctaccgccggtttatggtggtggatccaagttgacttggctgagatgtacaccgtgcgataacactcgatcaatctaccgccggtttatggtggtggcgtttgtgtgtgcatctgtaagcatgtacaccgtgcgataacactcgatcaatctaccgccggtttatggtggtggtgcacacttggacagcatttcctgtacaccgtgcgataacactcgatcaatctaccgccggtttatggtggtggaggaagggctgtgtttcagggctgtacaccgtgcgataacactcgatcaatctaccgccggtttatggtggtgcgcttttctggccagaaacctctgtacaccgtgcgataacactcgatcaatctaccgccggtttatggtggtgccagcttccctgattcttcagcttgtacaccgtgcgataacactcgatcaatctaccgccggtttatggtggtgaaaattaacttctctggtgtgtggagatgtacaccgtgcgataacactcgatcaatctaccgccggtttatggtggtgcctaacctatcatccatccttatctcttgtacaccgtgcgataacactcgatcaatctaccgccggtttatggtggtgtgcacccaacattctaacaaaaggctgtacaccgtgcgataacactcgatcaatctaccgccggtttatggtggtgcaataggtttttaaggaacaggtggtgtacaccgtgcgataacactcgatcaatctaccgccggtttatggtggtggggtgattcccattggcctgtacaccgtgcgataacactcgatcaatctaccgccggtttatggtggtggaagtagctgctgagtgatttgtctgtacaccgtgcgataacactcgatcaatctaccgccggtttatggtggtgcctctccaccctatagaccctgtacaccgtgcgataacactcgatcaatctaccgccggtttatggtggtgctaattaaagtggtgtcccagataatctgtacaccgtgcgataacactcgatcaatctaccgccggtttatggtggtgctctgacccatctaacgcctatctgtacaccgtgcgataacactcgatcaatctaccgccggtttatggtggtgtgagtagctgggactacaggcatgtacaccgtgcgataacactcgatcaatctaccgccggtttatggtggtgtgacgcggtctccgcggtgtacaccgtgcgataacactcgatcaatctaccgccggtttatggtggtgctttttattaaatgctttccatgtatcaagttctgtacaccgtgcgataacactcgatcaatctaccgccggtttatggtggtgctttcccaattctccttcagtcctgtacaccgtgcgataacactcgatcaatctaccgccggtttatggtggtggatatcagggaagatgaaaaaagagactgtacaccgtgcgataacactcgatcaatctaccgccggtttatggtggtggtcccagaggcccttgtcagtgtacaccgtgcgataacactcgatcaatctaccgccggtttatggtggtgcctgggctctgtaaagaatagtgtacacgaaccgattggtgcagtcttc

SEQ ID NO: 9 - DNA, 1736 bp, Artificial Sequence
  misc_binding
acaccgtgcgataacactcgatcaatctgtcagctccgaagagcagtttgggtgacagagcaagaccctgtacaccgtgcgataacactcgatcaatctgtcagctccgaagagcagttgatccaagttgacttggctgagatgtacaccgtgcgataacactcgatcaatctgtcagctccgaagagcagttgcgtttgtgtgtgcatctgtaagcatgtacaccgtgcgataacactcgatcaatctgtcagctccgaagagcagttgtgcacacttggacagcatttcctgtacaccgtgcgataacactcgatcaatctgtcagctccgaagagcagttgaggaagggctgtgtttcagggctgtacaccgtgcgataacactcgatcaatctgtcagctccgaagagcagttcgcttttctggccagaaacctctgtacaccgtgcgataacactcgatcaatctgtcagctccgaagagcagttccagcttccctgattcttcagcttgtacaccgtgcgataacactcgatcaatctgtcagctccgaagagcagttaaaattaacttctctggtgtgtggagatgtacaccgtgcgataacactcgatcaatctgtcagctccgaagagcagttcctaacctatcatccatccttatctcttgtacaccgtgcgataacactcgatcaatctgtcagctccgaagagcagtttgcacccaacattctaacaaaaggctgtacaccgtgcgataacactcgatcaatctgtcagctccgaagagcagttcaataggtttttaaggaacaggtggtgtacaccgtgcgataacactcgatcaatctgtcagctccgaagagcagttgggtgattcccattggcctgtacaccgtgcgataacactcgatcaatctgtcagctccgaagagcagttgaagtagctgctgagtgatttgtctgtacaccgtgcgataacactcgatcaatctgtcagctccgaagagcagttcctctccaccctatagaccctgtacaccgtgcgataacactcgatcaatctgtcagctccgaagagcagttctaattaaagtggtgtcccagataatctgtacaccgtgcgataacactcgatcaatctgtcagctccgaagagcagttctctgacccatctaacgcctatctgtacaccgtgcgataacactcgatcaatctgtcagctccgaagagcagtttgagtagctgggactacaggcatgtacaccgtgcgataacactcgatcaatctgtcagctccgaagagcagtttgacgcggtctccgcggtgtacaccgtgcgataacactcgatcaatctgtcagctccgaagagcagttctttttattaaatgctttccatgtatcaagttctgtacaccgtgcgataacactcgatcaatctgtcagctccgaagagcagttctttcccaattctccttcagtcctgtacaccgtgcgataacactcgatcaatctgtcagctccgaagagcagttgatatcagggaagatgaaaaaagagactgtacaccgtgcgataacactcgatcaatctgtcagctccgaagagcagttgtcccagaggcccttgtcagtgtacaccgtgcgataacactcgatcaatctgtcagctccgaagagcagttcctgggctctgtaaagaatagtgtacacgaaccgattggtgcagtcttc

SEQ ID NO: 10 - DNA, 1736 bp, Artificial Sequence
  misc_binding
acaccgtgcgataacactcgatcaatcttcgtatgcaggcgtggtcgatgggtgacagagcaagaccctgtacaccgtgcgataacactcgatcaatcttcgtatgcaggcgtggtcgagatccaagttgacttggctgagatgtacaccgtgcgataacactcgatcaatcttcgtatgcaggcgtggtcgagcgtttgtgtgtgcatctgtaagcatgtacaccgtgcgataacactcgatcaatcttcgtatgcaggcgtggtcgagtgcacacttggacagcatttcctgtacaccgtgcgataacactcgatcaatcttcgtatgcaggcgtggtcgagaggaagggctgtgtttcagggctgtacaccgtgcgataacactcgatcaatcttcgtatgcaggcgtggtcgacgcttttctggccagaaacctctgtacaccgtgcgataacactcgatcaatcttcgtatgcaggcgtggtcgaccagcttccctgattcttcagcttgtacaccgtgcgataacactcgatcaatcttcgtatgcaggcgtggtcgaaaaattaacttctctggtgtgtggagatgtacaccgtgcgataacactcgatcaatcttcgtatgcaggcgtggtcgacctaacctatcatccatccttatctcttgtacaccgtgcgataacactcgatcaatcttcgtatgcaggcgtggtcgatgcacccaacattctaacaaaaggctgtacaccgtgcgataacactcgatcaatcttcgtatgcaggcgtggtcgacaataggtttttaaggaacaggtggtgtacaccgtgcgataacactcgatcaatcttcgtatgcaggcgtggtcgagggtgattcccattggcctgtacaccgtgcgataacactcgatcaatcttcgtatgcaggcgtggtcgagaagtagctgctgagtgatttgtctgtacaccgtgcgataacactcgatcaatcttcgtatgcaggcgtggtcgacctctccaccctatagaccctgtacaccgtgcgataacactcgatcaatcttcgtatgcaggcgtggtcgactaattaaagtggtgtcccagataatctgtacaccgtgcgataacactcgatcaatcttcgtatgcaggcgtggtcgactctgacccatctaacgcctatctgtacaccgtgcgataacactcgatcaatcttcgtatgcaggcgtggtcgatgagtagctgggactacaggcatgtacaccgtgcgataacactcgatcaatcttcgtatgcaggcgtggtcgatgacgcggtctccgcggtgtacaccgtgcgataacactcgatcaatcttcgtatgcaggcgtggtcgactttttattaaatgctttccatgtatcaagttctgtacaccgtgcgataacactcgatcaatcttcgtatgcaggcgtggtcgactttcccaattctccttcagtcctgtacaccgtgcgataacactcgatcaatcttcgtatgcaggcgtggtcgagatatcagggaagatgaaaaaagagactgtacaccgtgcgataacactcgatcaatcttcgtatgcaggcgtggtcgagtcccagaggcccttgtcagtgtacaccgtgcgataacactcgatcaatcttcgtatgcaggcgtggtcgacctgggctctgtaaagaatagtgtacacgaaccgattggtgcagtcttc

SEQ ID NO: 11 - DNA, 1736 bp, Artificial Sequence
  misc_binding
acaccgtgcgataacactcgatcaatctgctcacggatatgcggttcatgggtgacagagcaagaccctgtacaccgtgcgataacactcgatcaatctgctcacggatatgcggttcagatccaagttgacttggctgagatgtacaccgtgcgataacactcgatcaatctgctcacggatatgcggttcagcgtttgtgtgtgcatctgtaagcatgtacaccgtgcgataacactcgatcaatctgctcacggatatgcggttcagtgcacacttggacagcatttcctgtacaccgtgcgataacactcgatcaatctgctcacggatatgcggttcagaggaagggctgtgtttcagggctgtacaccgtgcgataacactcgatcaatctgctcacggatatgcggttcacgcttttctggccagaaacctctgtacaccgtgcgataacactcgatcaatctgctcacggatatgcggttcaccagcttccctgattcttcagcttgtacaccgtgcgataacactcgatcaatctgctcacggatatgcggttcaaaaattaacttctctggtgtgtggagatgtacaccgtgcgataacactcgatcaatctgctcacggatatgcggttcacctaacctatcatccatccttatctcttgtacaccgtgcgataacactcgatcaatctgctcacggatatgcggttcatgcacccaacattctaacaaaaggctgtacaccgtgcgataacactcgatcaatctgctcacggatatgcggttcacaataggtttttaaggaacaggtggtgtacaccgtgcgataacactcgatcaatctgctcacggatatgcggttcagggtgattcccattggcctgtacaccgtgcgataacactcgatcaatctgctcacggatatgcggttcagaagtagctgctgagtgatttgtctgtacaccgtgcgataacactcgatcaatctgctcacggatatgcggttcacctctccaccctatagaccctgtacaccgtgcgataacactcgatcaatctgctcacggatatgcggttcactaattaaagtggtgtcccagataatctgtacaccgtgcgataacactcgatcaatctgctcacggatatgcggttcactctgacccatctaacgcctatctgtacaccgtgcgataacactcgatcaatctgctcacggatatgcggttcatgagtagctgggactacaggcatgtacaccgtgcgataacactcgatcaatctgctcacggatatgcggttcatgacgcggtctccgcggtgtacaccgtgcgataacactcgatcaatctgctcacggatatgcggttcactttttattaaatgctttccatgtatcaagttctgtacaccgtgcgataacactcgatcaatctgctcacggatatgcggttcactttcccaattctccttcagtcctgtacaccgtgcgataacactcgatcaatctgctcacggatatgcggttcagatatcagggaagatgaaaaaagagactgtacaccgtgcgataacactcgatcaatctgctcacggatatgcggttcagtcccagaggcccttgtcagtgtacaccgtgcgataacactcgatcaatctgctcacggatatgcggttcacctgggctctgtaaagaatagtgtacacgaaccgattggtgcagtcttc

SEQ ID NO: 12 - DNA, 1736 bp, Artificial Sequence
  misc_binding
acaccgtgcgataacactcgatcaatctatgcgtgacgctaaactgcttgggtgacagagcaagaccctgtacaccgtgcgataacactcgatcaatctatgcgtgacgctaaactgctgatccaagttgacttggctgagatgtacaccgtgcgataacactcgatcaatctatgcgtgacgctaaactgctgcgtttgtgtgtgcatctgtaagcatgtacaccgtgcgataacactcgatcaatctatgcgtgacgctaaactgctgtgcacacttggacagcatttcctgtacaccgtgcgataacactcgatcaatctatgcgtgacgctaaactgctgaggaagggctgtgtttcagggctgtacaccgtgcgataacactcgatcaatctatgcgtgacgctaaactgctcgcttttctggccagaaacctctgtacaccgtgcgataacactcgatcaatctatgcgtgacgctaaactgctccagcttccctgattcttcagcttgtacaccgtgcgataacactcgatcaatctatgcgtgacgctaaactgctaaaattaacttctctggtgtgtggagatgtacaccgtgcgataacactcgatcaatctatgcgtgacgctaaactgctcctaacctatcatccatccttatctcttgtacaccgtgcgataacactcgatcaatctatgcgtgacgctaaactgcttgcacccaacattctaacaaaaggctgtacaccgtgcgataacactcgatcaatctatgcgtgacgctaaactgctcaataggtttttaaggaacaggtggtgtacaccgtgcgataacactcgatcaatctatgcgtgacgctaaactgctgggtgattcccattggcctgtacaccgtgcgataacactcgatcaatctatgcgtgacgctaaactgctgaagtagctgctgagtgatttgtctgtacaccgtgcgataacactcgatcaatctatgcgtgacgctaaactgctcctctccaccctatagaccctgtacaccgtgcgataacactcgatcaatctatgcgtgacgctaaactgctctaattaaagtggtgtcccagataatctgtacaccgtgcgataacactcgatcaatctatgcgtgacgctaaactgctctctgacccatctaacgcctatctgtacaccgtgcgataacactcgatcaatctatgcgtgacgctaaactgcttgagtagctgggactacaggcatgtacaccgtgcgataacactcgatcaatctatgcgtgacgctaaactgcttgacgcggtctccgcggtgtacaccgtgcgataacactcgatcaatctatgcgtgacgctaaactgctctttttattaaatgctttccatgtatcaagttctgtacaccgtgcgataacactcgatcaatctatgcgtgacgctaaactgctctttcccaattctccttcagtcctgtacaccgtgcgataacactcgatcaatctatgcgtgacgctaaactgctgatatcagggaagatgaaaaaagagactgtacaccgtgcgataacactcgatcaatctatgcgtgacgctaaactgctgtcccagaggcccttgtcagtgtacaccgtgcgataacactcgatcaatctatgcgtgacgctaaactgctcctgggctctgtaaagaatagtgtacacgaaccgattggtgcagtcttc

SEQ ID NO: 13 - DNA, 1736 bp, Artificial Sequence
  misc_binding
acaccgtgcgataacactcgatcaatctgtgtcttcgcgtttacggactgggtgacagagcaagaccctgtacaccgtgcgataacactcgatcaatctgtgtcttcgcgtttacggacgatccaagttgacttggctgagatgtacaccgtgcgataacactcgatcaatctgtgtcttcgcgtttacggacgcgtttgtgtgtgcatctgtaagcatgtacaccgtgcgataacactcgatcaatctgtgtcttcgcgtttacggacgtgcacacttggacagcatttcctgtacaccgtgcgataacactcgatcaatctgtgtcttcgcgtttacggacgaggaagggctgtgtttcagggctgtacaccgtgcgataacactcgatcaatctgtgtcttcgcgtttacggaccgcttttctggccagaaacctctgtacaccgtgcgataacactcgatcaatctgtgtcttcgcgtttacggacccagcttccctgattcttcagcttgtacaccgtgcgataacactcgatcaatctgtgtcttcgcgtttacggacaaaattaacttctctggtgtgtggagatgtacaccgtgcgataacactcgatcaatctgtgtcttcgcgtttacggaccctaacctatcatccatccttatctcttgtacaccgtgcgataacactcgatcaatctgtgtcttcgcgtttacggactgcacccaacattctaacaaaaggctgtacaccgtgcgataacactcgatcaatctgtgtcttcgcgtttacggaccaataggtttttaaggaacaggtggtgtacaccgtgcgataacactcgatcaatctgtgtcttcgcgtttacggacgggtgattcccattggcctgtacaccgtgcgataacactcgatcaatctgtgtcttcgcgtttacggacgaagtagctgctgagtgatttgtctgtacaccgtgcgataacactcgatcaatctgtgtcttcgcgtttacggaccctctccaccctatagaccctgtacaccgtgcgataacactcgatcaatctgtgtcttcgcgtttacggacctaattaaagtggtgtcccagataatctgtacaccgtgcgataacactcgatcaatctgtgtcttcgcgtttacggacctctgacccatctaacgcctatctgtacaccgtgcgataacactcgatcaatctgtgtcttcgcgtttacggactgagtagctgggactacaggcatgtacaccgtgcgataacactcgatcaatctgtgtcttcgcgtttacggactgacgcggtctccgcggtgtacaccgtgcgataacactcgatcaatctgtgtcttcgcgtttacggacctttttattaaatgctttccatgtatcaagttctgtacaccgtgcgataacactcgatcaatctgtgtcttcgcgtttacggacctttcccaattctccttcagtcctgtacaccgtgcgataacactcgatcaatctgtgtcttcgcgtttacggacgatatcagggaagatgaaaaaagagactgtacaccgtgcgataacactcgatcaatctgtgtcttcgcgtttacggacgtcccagaggcccttgtcagtgtacaccgtgcgataacactcgatcaatctgtgtcttcgcgtttacggaccctgggctctgtaaagaatagtgtacacgaaccgattggtgcagtcttc

SEQ ID NO: 14 - DNA, 1736 bp, Artificial Sequence
  misc_binding
acaccgtgcgataacactcgatcaatctctgatgcgactaacataatatgggtgacagagcaagaccctgtacaccgtgcgataacactcgatcaatctctgatgcgactaacataatagatccaagttgacttggctgagatgtacaccgtgcgataacactcgatcaatctctgatgcgactaacataatagcgtttgtgtgtgcatctgtaagcatgtacaccgtgcgataacactcgatcaatctctgatgcgactaacataatagtgcacacttggacagcatttcctgtacaccgtgcgataacactcgatcaatctctgatgcgactaacataatagaggaagggctgtgtttcagggctgtacaccgtgcgataacactcgatcaatctctgatgcgactaacataatacgcttttctggccagaaacctctgtacaccgtgcgataacactcgatcaatctctgatgcgactaacataataccagcttccctgattcttcagcttgtacaccgtgcgataacactcgatcaatctctgatgcgactaacataataaaaattaacttctctggtgtgtggagatgtacaccgtgcgataacactcgatcaatctctgatgcgactaacataatacctaacctatcatccatccttatctcttgtacaccgtgcgataacactcgatcaatctctgatgcgactaacataatatgcacccaacattctaacaaaaggctgtacaccgtgcgataacactcgatcaatctctgatgcgactaacataatacaataggtttttaaggaacaggtggtgtacaccgtgcgataacactcgatcaatctctgatgcgactaacataatagggtgattcccattggcctgtacaccgtgcgataacactcgatcaatctctgatgcgactaacataatagaagtagctgctgagtgatttgtctgtacaccgtgcgataacactcgatcaatctctgatgcgactaacataatacctctccaccctatagaccctgtacaccgtgcgataacactcgatcaatctctgatgcgactaacataatactaattaaagtggtgtcccagataatctgtacaccgtgcgataacactcgatcaatctctgatgcgactaacataatactctgacccatctaacgcctatctgtacaccgtgcgataacactcgatcaatctctgatgcgactaacataatatgagtagctgggactacaggcatgtacaccgtgcgataacactcgatcaatctctgatgcgactaacataatatgacgcggtctccgcggtgtacaccgtgcgataacactcgatcaatctctgatgcgactaacataatactttttattaaatgctttccatgtatcaagttctgtacaccgtgcgataacactcgatcaatctctgatgcgactaacataatactttcccaattctccttcagtcctgtacaccgtgcgataacactcgatcaatctctgatgcgactaacataatagatatcagggaagatgaaaaaagagactgtacaccgtgcgataacactcgatcaatctctgatgcgactaacataatagtcccagaggcccttgtcagtgtacaccgtgcgataacactcgatcaatctctgatgcgactaacataatacctgggctctgtaaagaatagtgtacacgaaccgattggtgcagtcttc

SEQ ID NO: 15 - DNA, 1736 bp, Artificial Sequence
  misc_binding
acaccgtgcgataacactcgatcaatctaacgaccccgtccatgggtatgggtgacagagcaagaccctgtacaccgtgcgataacactcgatcaatctaacgaccccgtccatgggtagatccaagttgacttggctgagatgtacaccgtgcgataacactcgatcaatctaacgaccccgtccatgggtagcgtttgtgtgtgcatctgtaagcatgtacaccgtgcgataacactcgatcaatctaacgaccccgtccatgggtagtgcacacttggacagcatttcctgtacaccgtgcgataacactcgatcaatctaacgaccccgtccatgggtagaggaagggctgtgtttcagggctgtacaccgtgcgataacactcgatcaatctaacgaccccgtccatgggtacgcttttctggccagaaacctctgtacaccgtgcgataacactcgatcaatctaacgaccccgtccatgggtaccagcttccctgattcttcagcttgtacaccgtgcgataacactcgatcaatctaacgaccccgtccatgggtaaaaattaacttctctggtgtgtggagatgtacaccgtgcgataacactcgatcaatctaacgaccccgtccatgggtacctaacctatcatccatccttatctcttgtacaccgtgcgataacactcgatcaatctaacgaccccgtccatgggtatgcacccaacattctaacaaaaggctgtacaccgtgcgataacactcgatcaatctaacgaccccgtccatgggtacaataggtttttaaggaacaggtggtgtacaccgtgcgataacactcgatcaatctaacgaccccgtccatgggtagggtgattcccattggcctgtacaccgtgcgataacactcgatcaatctaacgaccccgtccatgggtagaagtagctgctgagtgatttgtctgtacaccgtgcgataacactcgatcaatctaacgaccccgtccatgggtacctctccaccctatagaccctgtacaccgtgcgataacactcgatcaatctaacgaccccgtccatgggtactaattaaagtggtgtcccagataatctgtacaccgtgcgataacactcgatcaatctaacgaccccgtccatgggtactctgacccatctaacgcctatctgtacaccgtgcgataacactcgatcaatctaacgaccccgtccatgggtatgagtagctgggactacaggcatgtacaccgtgcgataacactcgatcaatctaacgaccccgtccatgggtatgacgcggtctccgcggtgtacaccgtgcgataacactcgatcaatctaacgaccccgtccatgggtactttttattaaatgctttccatgtatcaagttctgtacaccgtgcgataacactcgatcaatctaacgaccccgtccatgggtactttcccaattctccttcagtcctgtacaccgtgcgataacactcgatcaatctaacgaccccgtccatgggtagatatcagggaagatgaaaaaagagactgtacaccgtgcgataacactcgatcaatctaacgaccccgtccatgggtagtcccagaggcccttgtcagtgtacaccgtgcgataacactcgatcaatctaacgaccccgtccatgggtacctgggctctgtaaagaatagtgtacacgaaccgattggtgcagtcttc

SEQ ID NO: 16 - DNA, 1736 bp, Artificial Sequence
  misc_binding
acaccgtgcgataacactcgatcaatcttctttacgcgttgattcctatgggtgacagagcaagaccctgtacaccgtgcgataacactcgatcaatcttctttacgcgttgattcctagatccaagttgacttggctgagatgtacaccgtgcgataacactcgatcaatcttctttacgcgttgattcctagcgtttgtgtgtgcatctgtaagcatgtacaccgtgcgataacactcgatcaatcttctttacgcgttgattcctagtgcacacttggacagcatttcctgtacaccgtgcgataacactcgatcaatcttctttacgcgttgattcctagaggaagggctgtgtttcagggctgtacaccgtgcgataacactcgatcaatcttctttacgcgttgattcctacgcttttctggccagaaacctctgtacaccgtgcgataacactcgatcaatcttctttacgcgttgattcctaccagcttccctgattcttcagcttgtacaccgtgcgataacactcgatcaatcttctttacgcgttgattcctaaaaattaacttctctggtgtgtggagatgtacaccgtgcgataacactcgatcaatcttctttacgcgttgattcctacctaacctatcatccatccttatctcttgtacaccgtgcgataacactcgatcaatcttctttacgcgttgattcctatgcacccaacattctaacaaaaggctgtacaccgtgcgataacactcgatcaatcttctttacgcgttgattcctacaataggtttttaaggaacaggtggtgtacaccgtgcgataacactcgatcaatcttctttacgcgttgattcctagggtgattcccattggcctgtacaccgtgcgataacactcgatcaatcttctttacgcgttgattcctagaagtagctgctgagtgatttgtctgtacaccgtgcgataacactcgatcaatcttctttacgcgttgattcctacctctccaccctatagaccctgtacaccgtgcgataacactcgatcaatcttctttacgcgttgattcctactaattaaagtggtgtcccagataatctgtacaccgtgcgataacactcgatcaatcttctttacgcgttgattcctactctgacccatctaacgcctatctgtacaccgtgcgataacactcgatcaatcttctttacgcgttgattcctatgagtagctgggactacaggcatgtacaccgtgcgataacactcgatcaatcttctttacgcgttgattcctatgacgcggtctccgcggtgtacaccgtgcgataacactcgatcaatcttctttacgcgttgattcctactttttattaaatgctttccatgtatcaagttctgtacaccgtgcgataacactcgatcaatcttctttacgcgttgattcctactttcccaattctccttcagtcctgtacaccgtgcgataacactcgatcaatcttctttacgcgttgattcctagatatcagggaagatgaaaaaagagactgtacaccgtgcgataacactcgatcaatcttctttacgcgttgattcctagtcccagaggcccttgtcagtgtacaccgtgcgataacactcgatcaatcttctttacgcgttgattcctacctgggctctgtaaagaatagtgtacacgaaccgattggtgcagtcttc

SEQ ID NO: 17 - DNA, 1736 bp, Artificial Sequence
  misc_binding
acaccgtgcgataacactcgatcaatctcctagtagtgacctcccgtctgggtgacagagcaagaccctgtacaccgtgcgataacactcgatcaatctcctagtagtgacctcccgtcgatccaagttgacttggctgagatgtacaccgtgcgataacactcgatcaatctcctagtagtgacctcccgtcgcgtttgtgtgtgcatctgtaagcatgtacaccgtgcgataacactcgatcaatctcctagtagtgacctcccgtcgtgcacacttggacagcatttcctgtacaccgtgcgataacactcgatcaatctcctagtagtgacctcccgtcgaggaagggctgtgtttcagggctgtacaccgtgcgataacactcgatcaatctcctagtagtgacctcccgtccgcttttctggccagaaacctctgtacaccgtgcgataacactcgatcaatctcctagtagtgacctcccgtcccagcttccctgattcttcagcttgtacaccgtgcgataacactcgatcaatctcctagtagtgacctcccgtcaaaattaacttctctggtgtgtggagatgtacaccgtgcgataacactcgatcaatctcctagtagtgacctcccgtccctaacctatcatccatccttatctcttgtacaccgtgcgataacactcgatcaatctcctagtagtgacctcccgtctgcacccaacattctaacaaaaggctgtacaccgtgcgataacactcgatcaatctcctagtagtgacctcccgtccaataggtttttaaggaacaggtggtgtacaccgtgcgataacactcgatcaatctcctagtagtgacctcccgtcgggtgattcccattggcctgtacaccgtgcgataacactcgatcaatctcctagtagtgacctcccgtcgaagtagctgctgagtgatttgtctgtacaccgtgcgataacactcgatcaatctcctagtagtgacctcccgtccctctccaccctatagaccctgtacaccgtgcgataacactcgatcaatctcctagtagtgacctcccgtcctaattaaagtggtgtcccagataatctgtacaccgtgcgataacactcgatcaatctcctagtagtgacctcccgtcctctgacccatctaacgcctatctgtacaccgtgcgataacactcgatcaatctcctagtagtgacctcccgtctgagtagctgggactacaggcatgtacaccgtgcgataacactcgatcaatctcctagtagtgacctcccgtctgacgcggtctccgcggtgtacaccgtgcgataacactcgatcaatctcctagtagtgacctcccgtcctttttattaaatgctttccatgtatcaagttctgtacaccgtgcgataacactcgatcaatctcctagtagtgacctcccgtcctttcccaattctccttcagtcctgtacaccgtgcgataacactcgatcaatctcctagtagtgacctcccgtcgatatcagggaagatgaaaaaagagactgtacaccgtgcgataacactcgatcaatctcctagtagtgacctcccgtcgtcccagaggcccttgtcagtgtacaccgtgcgataacactcgatcaatctcctagtagtgacctcccgtccctgggctctgtaaagaatagtgtacacgaaccgattggtgcagtcttc

SEQ ID NO: 18 - DNA, 1736 bp, Artificial Sequence
  misc_binding
acaccgtgcgataacactcgatcaatctatgcggtataatatgttggctgggtgacagagcaagaccctgtacaccgtgcgataacactcgatcaatctatgcggtataatatgttggcgatccaagttgacttggctgagatgtacaccgtgcgataacactcgatcaatctatgcggtataatatgttggcgcgtttgtgtgtgcatctgtaagcatgtacaccgtgcgataacactcgatcaatctatgcggtataatatgttggcgtgcacacttggacagcatttcctgtacaccgtgcgataacactcgatcaatctatgcggtataatatgttggcgaggaagggctgtgtttcagggctgtacaccgtgcgataacactcgatcaatctatgcggtataatatgttggccgcttttctggccagaaacctctgtacaccgtgcgataacactcgatcaatctatgcggtataatatgttggcccagcttccctgattcttcagcttgtacaccgtgcgataacactcgatcaatctatgcggtataatatgttggcaaaattaacttctctggtgtgtggagatgtacaccgtgcgataacactcgatcaatctatgcggtataatatgttggccctaacctatcatccatccttatctcttgtacaccgtgcgataacactcgatcaatctatgcggtataatatgttggctgcacccaacattctaacaaaaggctgtacaccgtgcgataacactcgatcaatctatgcggtataatatgttggccaataggtttttaaggaacaggtggtgtacaccgtgcgataacactcgatcaatctatgcggtataatatgttggcgggtgattcccattggcctgtacaccgtgcgataacactcgatcaatctatgcggtataatatgttggcgaagtagctgctgagtgatttgtctgtacaccgtgcgataacactcgatcaatctatgcggtataatatgttggccctctccaccctatagaccctgtacaccgtgcgataacactcgatcaatctatgcggtataatatgttggcctaattaaagtggtgtcccagataatctgtacaccgtgcgataacactcgatcaatctatgcggtataatatgttggcctctgacccatctaacgcctatctgtacaccgtgcgataacactcgatcaatctatgcggtataatatgttggctgagtagctgggactacaggcatgtacaccgtgcgataacactcgatcaatctatgcggtataatatgttggctgacgcggtctccgcggtgtacaccgtgcgataacactcgatcaatctatgcggtataatatgttggcctttttattaaatgctttccatgtatcaagttctgtacaccgtgcgataacactcgatcaatctatgcggtataatatgttggcctttcccaattctccttcagtcctgtacaccgtgcgataacactcgatcaatctatgcggtataatatgttggcgatatcagggaagatgaaaaaagagactgtacaccgtgcgataacactcgatcaatctatgcggtataatatgttggcgtcccagaggcccttgtcagtgtacaccgtgcgataacactcgatcaatctatgcggtataatatgttggccctgggctctgtaaagaatagtgtacacgaaccgattggtgcagtcttc

SEQ ID NO: 19 - DNA, 1736 bp, Artificial Sequence
  misc_binding
acaccgtgcgataacactcgatcaatctactttgaatccaagcattgttgggtgacagagcaagaccctgtacaccgtgcgataacactcgatcaatctactttgaatccaagcattgtgatccaagttgacttggctgagatgtacaccgtgcgataacactcgatcaatctactttgaatccaagcattgtgcgtttgtgtgtgcatctgtaagcatgtacaccgtgcgataacactcgatcaatctactttgaatccaagcattgtgtgcacacttggacagcatttcctgtacaccgtgcgataacactcgatcaatctactttgaatccaagcattgtgaggaagggctgtgtttcagggctgtacaccgtgcgataacactcgatcaatctactttgaatccaagcattgtcgcttttctggccagaaacctctgtacaccgtgcgataacactcgatcaatctactttgaatccaagcattgtccagcttccctgattcttcagcttgtacaccgtgcgataacactcgatcaatctactttgaatccaagcattgtaaaattaacttctctggtgtgtggagatgtacaccgtgcgataacactcgatcaatctactttgaatccaagcattgtcctaacctatcatccatccttatctcttgtacaccgtgcgataacactcgatcaatctactttgaatccaagcattgttgcacccaacattctaacaaaaggctgtacaccgtgcgataacactcgatcaatctactttgaatccaagcattgtcaataggtttttaaggaacaggtggtgtacaccgtgcgataacactcgatcaatctactttgaatccaagcattgtgggtgattcccattggcctgtacaccgtgcgataacactcgatcaatctactttgaatccaagcattgtgaagtagctgctgagtgatttgtctgtacaccgtgcgataacactcgatcaatctactttgaatccaagcattgtcctctccaccctatagaccctgtacaccgtgcgataacactcgatcaatctactttgaatccaagcattgtctaattaaagtggtgtcccagataatctgtacaccgtgcgataacactcgatcaatctactttgaatccaagcattgtctctgacccatctaacgcctatctgtacaccgtgcgataacactcgatcaatctactttgaatccaagcattgttgagtagctgggactacaggcatgtacaccgtgcgataacactcgatcaatctactttgaatccaagcattgttgacgcggtctccgcggtgtacaccgtgcgataacactcgatcaatctactttgaatccaagcattgtctttttattaaatgctttccatgtatcaagttctgtacaccgtgcgataacactcgatcaatctactttgaatccaagcattgtctttcccaattctccttcagtcctgtacaccgtgcgataacactcgatcaatctactttgaatccaagcattgtgatatcagggaagatgaaaaaagagactgtacaccgtgcgataacactcgatcaatctactttgaatccaagcattgtgtcccagaggcccttgtcagtgtacaccgtgcgataacactcgatcaatctactttgaatccaagcattgtcctgggctctgtaaagaatagtgtacacgaaccgattggtgcagtcttc

SEQ ID NO: 20 - DNA, 1736 bp, Artificial Sequence
  misc_binding
acaccgtgcgataacactcgatcaatctccaggagtgcgggcccaccatgggtgacagagcaagaccctgtacaccgtgcgataacactcgatcaatctccaggagtgcgggcccaccagatccaagttgacttggctgagatgtacaccgtgcgataacactcgatcaatctccaggagtgcgggcccaccagcgtttgtgtgtgcatctgtaagcatgtacaccgtgcgataacactcgatcaatctccaggagtgcgggcccaccagtgcacacttggacagcatttcctgtacaccgtgcgataacactcgatcaatctccaggagtgcgggcccaccagaggaagggctgtgtttcagggctgtacaccgtgcgataacactcgatcaatctccaggagtgcgggcccaccacgcttttctggccagaaacctctgtacaccgtgcgataacactcgatcaatctccaggagtgcgggcccaccaccagcttccctgattcttcagcttgtacaccgtgcgataacactcgatcaatctccaggagtgcgggcccaccaaaaattaacttctctggtgtgtggagatgtacaccgtgcgataacactcgatcaatctccaggagtgcgggcccaccacctaacctatcatccatccttatctcttgtacaccgtgcgataacactcgatcaatctccaggagtgcgggcccaccatgcacccaacattctaacaaaaggctgtacaccgtgcgataacactcgatcaatctccaggagtgcgggcccaccacaataggtttttaaggaacaggtggtgtacaccgtgcgataacactcgatcaatctccaggagtgcgggcccaccagggtgattcccattggcctgtacaccgtgcgataacactcgatcaatctccaggagtgcgggcccaccagaagtagctgctgagtgatttgtctgtacaccgtgcgataacactcgatcaatctccaggagtgcgggcccaccacctctccaccctatagaccctgtacaccgtgcgataacactcgatcaatctccaggagtgcgggcccaccactaattaaagtggtgtcccagataatctgtacaccgtgcgataacactcgatcaatctccaggagtgcgggcccaccactctgacccatctaacgcctatctgtacaccgtgcgataacactcgatcaatctccaggagtgcgggcccaccatgagtagctgggactacaggcatgtacaccgtgcgataacactcgatcaatctccaggagtgcgggcccaccatgacgcggtctccgcggtgtacaccgtgcgataacactcgatcaatctccaggagtgcgggcccaccactttttattaaatgctttccatgtatcaagttctgtacaccgtgcgataacactcgatcaatctccaggagtgcgggcccaccactttcccaattctccttcagtcctgtacaccgtgcgataacactcgatcaatctccaggagtgcgggcccaccagatatcagggaagatgaaaaaagagactgtacaccgtgcgataacactcgatcaatctccaggagtgcgggcccaccagtcccagaggcccttgtcagtgtacaccgtgcgataacactcgatcaatctccaggagtgcgggcccaccacctgggctctgtaaagaatagtgtacacgaaccgattggtgcagtcttc

SEQ ID NO: 21 - DNA, 23 bp, Artificial Sequence
  primer_bind
tgggtgacagagcaagaccctgt

SEQ ID NO: 22 - DNA, 26 bp, Artificial Sequence
  primer_bind
gatccaagttgacttggctgagatgt

SEQ ID NO: 23 - DNA, 25 bp, Artificial Sequence
  primer_bind
tgtacaagtgccagatgctcgttgt

SEQ ID NO: 24 - DNA, 26 bp, Artificial Sequence
  primer_bind
gtgcacacttggacagcatttcctgt

SEQ ID NO: 25 - DNA, 26 bp, Artificial Sequence
  primer_bind
gaggaagggctgtgtttcagggctgt

SEQ ID NO: 26 - DNA, 25 bp, Artificial Sequence
  primer_bind
cgcttttctggccagaaacctctgt

SEQ ID NO: 27 - DNA, 30 bp, Artificial Sequence
  primer_bind
cctaacctatcatccatccttatctcttgt

SEQ ID NO: 28 - DNA, 28 bp, Artificial Sequence
  primer_bind
tgcacccaacattctaacaaaaggctgt

SEQ ID NO: 29 - DNA, 28 bp, Artificial Sequence
  primer_bind
caataggtttttaaggaacaggtggtgt

SEQ ID NO: 30 - DNA, 21 bp, Artificial Sequence
  primer_bind
gggtgattcccattggcctgt

SEQ ID NO: 31 - DNA, 27 bp, Artificial Sequence
  primer_bind
gaagtagctgctgagtgatttgtctgt

SEQ ID NO: 32 - DNA, 23 bp, Artificial Sequence
  primer_bind
cctctccaccctatagaccctgt

SEQ ID NO: 33 - DNA, 30 bp, Artificial Sequence
  primer_bind
ctaattaaagtggtgtcccagataatctgt

SEQ ID NO: 34 - DNA, 26 bp, Artificial Sequence
  primer_bind
ctctgacccatctaacgcctatctgt

SEQ ID NO: 35 - DNA, 25 bp, Artificial Sequence
  primer_bind
tgagtagctgggactacaggcatgt

SEQ ID NO: 36 - DNA, 20 bp, Artificial Sequence
  primer_bind
tgacgcggtctccgcggtgt

SEQ ID NO: 37 - DNA, 36 bp, Artificial Sequence
  primer_bind
ctttttattaaatgctttccatgtatcaagttctgt

SEQ ID NO: 38 - DNA, 26 bp, Artificial Sequence
  primer_bind
ctttcccaattctccttcagtcctgt

SEQ ID NO: 39 - DNA, 30 bp, Artificial Sequence
  primer_bind
gatatcagggaagatgaaaaaagagactgt

SEQ ID NO: 40 - DNA, 23 bp, Artificial Sequence
  primer_bind
gtcccagaggcccttgtcagtgt

SEQ ID NO: 41 - DNA, 21 bp, Artificial Sequence
  primer_bind
gtgtattccctgtgcctttgg

SEQ ID NO: 42 - DNA, 27 bp, Artificial Sequence
  primer_bind
agataggacagatgataaatacatagg

SEQ ID NO: 43 - DNA, 26 bp, Artificial Sequence
  primer_bind
cctcttccctagatcaatacagacag

SEQ ID NO: 44 - DNA, 23 bp, Artificial Sequence
  primer_bind
agggcagtgttccaacctgagtc

SEQ ID NO: 45 - DNA, 23 bp, Artificial Sequence
  primer_bind
gctgtgcgctggtcttactcctg

SEQ ID NO: 46 - DNA, 25 bp, Artificial Sequence
  primer_bind
ttcatcactgtatcgtatcccattg

SEQ ID NO: 47 - DNA, 27 bp, Artificial Sequence
  primer_bind
gatgttgtattagtcaatgttctccag

SEQ ID NO: 48 - DNA, 24 bp, Artificial Sequence
  primer_bind
tgttcatgccactgcacttcactc

SEQ ID NO: 49 - DNA, 25 bp, Artificial Sequence
  primer_bind
tggttgcaagcaattgccatagagg

SEQ ID NO: 50 - DNA, 21 bp, Artificial Sequence
  primer_bind
tggagctaagtggctgtggtg

SEQ ID NO: 51 - DNA, 23 bp, Artificial Sequence
  primer_bind
aggaggttgaggctgcaaaaagc

SEQ ID NO: 52 - DNA, 23 bp, Artificial Sequence
  primer_bind
gaaaagctcccgattatccagcc

SEQ ID NO: 53 - DNA, 24 bp, Artificial Sequence
  primer_bind
gccccataggttttgaactcacag

SEQ ID NO: 54 - DNA, 23 bp, Artificial Sequence
  primer_bind
agtgcctgtgcccaagttgagag

SEQ ID NO: 55 - DNA, 26 bp, Artificial Sequence
  primer_bind
ttagtgctttttagccaagtgattcc

SEQ ID NO: 56 - DNA, 22 bp, Artificial Sequence
  primer_bind
ggcagcccaaaaagacagacag

SEQ ID NO: 57 - DNA, 26 bp, Artificial Sequence
  primer_bind
tttatcctcattgacagaattgcacc

SEQ ID NO: 58 - DNA, 23 bp, Artificial Sequence
  primer_bind
aatctgggcgacaagagtgaaac

SEQ ID NO: 59 - DNA, 26 bp, Artificial Sequence
  primer_bind
ggaacaactctggttgtattgtcttc

SEQ ID NO: 60 - DNA, 22 bp, Artificial Sequence
  primer_bind
aagcctgtgttgctcaagggtc

SEQ ID NO: 61 - DNA, 26 bp, Artificial Sequence
  primer_bind
ctccatatcacttgagctaattcctc

SEQ ID NO: 62 - DNA, 26 bp, Artificial Sequence
  primer_bind
gtggatttggaaacagaaatggcttg

SEQ ID NO: 63 - DNA, 22 bp, Artificial Sequence
  primer_bind
gcttgaggccaaccatcagagc

SEQ ID NO: 64 - DNA, 33 bp, Artificial Sequence
  primer_bind
acactctttccctacacgacgctcttccgatct

SEQ ID NO: 65 - DNA, 20 bp, Artificial Sequence
  primer_bind
tcccgtctcaccggtctccc

SEQ ID NO: 66 - DNA, 23 bp, Artificial Sequence
  primer_bind
tgggtgacagagcaagaccctgt

SEQ ID NO: 67 - DNA, 33 bp, Artificial Sequence; identical to SEQ ID NO: 64
  primer_bind

SEQ ID NO: 68 - DNA, 20 bp, Artificial Sequence
  primer_bind
tcccgtctcaccggtctccc

SEQ ID NO: 69 - DNA, 26 bp, Artificial Sequence
  primer_bind
gatccaagttgacttggctgagatgt

SEQ ID NO: 70 - DNA, 33 bp, Artificial Sequence; identical to SEQ ID NO: 64
  primer_bind

SEQ ID NO: 71 - DNA, 20 bp, Artificial Sequence
  primer_bind
tcccgtctcaccggtctccc

SEQ ID NO: 72 - DNA, 25 bp, Artificial Sequence
  primer_bind
tgtacaagtgccagatgctcgttgt

SEQ ID NO: 73 - DNA, 33 bp, Artificial Sequence; identical to SEQ ID NO: 64
  primer_bind

SEQ ID NO: 74 - DNA, 20 bp, Artificial Sequence
  primer_bind
tcccgtctcaccggtctccc

SEQ ID NO: 75 - DNA, 26 bp, Artificial Sequence
  primer_bind
gtgcacacttggacagcatttcctgt

SEQ ID NO: 76 - DNA, 33 bp, Artificial Sequence; identical to SEQ ID NO: 64
  primer_bind

SEQ ID NO: 77 - DNA, 20 bp, Artificial Sequence
  primer_bind
tcccgtctcaccggtctccc

SEQ ID NO: 78 - DNA, 26 bp, Artificial Sequence
  primer_bind
gaggaagggctgtgtttcagggctgt

SEQ ID NO: 79 - DNA, 33 bp, Artificial Sequence; identical to SEQ ID NO: 64
  primer_bind

SEQ ID NO: 80 - DNA, 20 bp, Artificial Sequence
  primer_bind
tcccgtctcaccggtctccc

SEQ ID NO: 81 - DNA, 25 bp, Artificial Sequence
  primer_bind
cgcttttctggccagaaacctctgt

SEQ ID NO: 82 - DNA, 33 bp, Artificial Sequence; identical to SEQ ID NO: 64
  primer_bind

SEQ ID NO: 83 - DNA, 20 bp, Artificial Sequence
  primer_bind
tcccgtctcaccggtctccc

SEQ ID NO: 84 - DNA, 30 bp, Artificial Sequence; identical to SEQ ID NO: 27
  primer_bind

SEQ ID NO: 85 - DNA, 33 bp, Artificial Sequence; identical to SEQ ID NO: 64
  primer_bind

SEQ ID NO: 86 - DNA, 20 bp, Artificial Sequence
  primer_bind
tcccgtctcaccggtctccc

SEQ ID NO: 87 - DNA, 28 bp, Artificial Sequence
  primer_bind
tgcacccaacattctaacaaaaggctgt

SEQ ID NO: 88 - DNA, 33 bp, Artificial Sequence; identical to SEQ ID NO: 64
  primer_bind

SEQ ID NO: 89 - DNA, 20 bp, Artificial Sequence
  primer_bind
tcccgtctcaccggtctccc

SEQ ID NO: 90 - DNA, 28 bp, Artificial Sequence
  primer_bind
caataggtttttaaggaacaggtggtgt

SEQ ID NO: 91 - DNA, 33 bp, Artificial Sequence; identical to SEQ ID NO: 64
  primer_bind

SEQ ID NO: 92 - DNA, 20 bp, Artificial Sequence
  primer_bind
tcccgtctcaccggtctccc

SEQ ID NO: 93 - DNA, 21 bp, Artificial Sequence
  primer_bind
gggtgattcccattggcctgt

SEQ ID NO: 94 - DNA, 33 bp, Artificial Sequence; identical to SEQ ID NO: 64
  primer_bind

SEQ ID NO: 95 - DNA, 20 bp, Artificial Sequence
  primer_bind
tcccgtctcaccggtctccc

SEQ ID NO: 96 - DNA, 27 bp, Artificial Sequence
  primer_bind
gaagtagctgctgagtgatttgtctgt

SEQ ID NO: 97 - DNA, 33 bp, Artificial Sequence; identical to SEQ ID NO: 64
  primer_bind

SEQ ID NO: 98 - DNA, 20 bp, Artificial Sequence
  primer_bind
tcccgtctcaccggtctccc

SEQ ID NO: 99 - DNA, 23 bp, Artificial Sequence
  primer_bind
cctctccaccctatagaccctgt

SEQ ID NO: 100 - DNA, 33 bp, Artificial Sequence; identical to SEQ ID NO: 64
  primer_bind

SEQ ID NO: 101 - DNA, 20 bp, Artificial Sequence
  primer_bind
tcccgtctcaccggtctccc

SEQ ID NO: 102 - DNA, 30 bp, Artificial Sequence; identical to SEQ ID NO: 33
  primer_bind

SEQ ID NO: 103 - DNA, 33 bp, Artificial Sequence; identical to SEQ ID NO: 64
  primer_bind

SEQ ID NO: 104 - DNA, 20 bp, Artificial Sequence
  primer_bind
tcccgtctcaccggtctccc

SEQ ID NO: 105 - DNA, 26 bp, Artificial Sequence
  primer_bind
ctctgacccatctaacgcctatctgt

SEQ ID NO: 106 - DNA, 33 bp, Artificial Sequence; identical to SEQ ID NO: 64
  primer_bind

SEQ ID NO: 107 - DNA, 20 bp, Artificial Sequence
  primer_bind
tcccgtctcaccggtctccc

SEQ ID NO: 108 - DNA, 25 bp, Artificial Sequence
  primer_bind
tgagtagctgggactacaggcatgt

SEQ ID NO: 109 - DNA, 33 bp, Artificial Sequence; identical to SEQ ID NO: 64
  primer_bind

SEQ ID NO: 110 - DNA, 20 bp, Artificial Sequence
  primer_bind
tcccgtctcaccggtctccc

SEQ ID NO: 111 - DNA, 20 bp, Artificial Sequence
  primer_bind
tgacgcggtctccgcggtgt

SEQ ID NO: 112 - DNA, 33 bp, Artificial Sequence; identical to SEQ ID NO: 64
  primer_bind

SEQ ID NO: 113 - DNA, 20 bp, Artificial Sequence
  primer_bind
tcccgtctcaccggtctccc

SEQ ID NO: 114 - DNA, 36 bp, Artificial Sequence; identical to SEQ ID NO: 37
  primer_bind

SEQ ID NO: 115 - DNA, 33 bp, Artificial Sequence; identical to SEQ ID NO: 64
  primer_bind

SEQ ID NO: 116 - DNA, 20 bp, Artificial Sequence
  primer_bind
tcccgtctcaccggtctccc

SEQ ID NO: 117 - DNA, 26 bp, Artificial Sequence
  primer_bind
ctttcccaattctccttcagtcctgt

SEQ ID NO: 118 - DNA, 33 bp, Artificial Sequence; identical to SEQ ID NO: 64
  primer_bind

SEQ ID NO: 119 - DNA, 20 bp, Artificial Sequence
  primer_bind
tcccgtctcaccggtctccc

SEQ ID NO: 120 - DNA, 30 bp, Artificial Sequence; identical to SEQ ID NO: 39
  primer_bind

SEQ ID NO: 121 - DNA, 53 bp, Artificial Sequence
  primer_bind
acactctttccctacacgacgctcttccgatctggcgaaggaaagggcgacgg

SEQ ID NO: 122 - DNA, 20 bp, Artificial Sequence
  primer_bind
tcccgtctcaccggtctccc

SEQ ID NO: 123 - DNA, 23 bp, Artificial Sequence
  primer_bind
gtcccagaggcccttgtcagtgt

(19) United States
(12) Patent Application Publication
(10) Pub. No.: US 2023/0203475 A1
(43) Pub. Date: Jun. 29, 2023

(54) ENHANCEMENTS TO SINGLE CELL OR NUCLEUS NEXT GENERATION SEQUENCING FOR REDUCING COSTS AND IMPROVING THROUGHPUT

(71) Applicant: Tony Nick Frudakis, Lynn, MA (US)

(72) Inventor: Tony Nick Frudakis, Lynn, MA (US)

(21) Appl. No.: 17/560,378

(22) Filed: Dec. 23, 2021

(51) Int. Cl. C12N 15/10; C40B 40/06

(52) U.S. Cl. CPC C12N 15/1013; C40B 40/06; C12N 2320/12; C07B 2200/11; C12Q 2563/179; C12Q 2563/185

(57) ABSTRACT

According to some aspects of the present disclosure, a platform and a set of consumables and reagents are provided to seamlessly integrate with and elegantly transform integrated Single Cell Next Generation Sequencing (SCNGS) platforms for dramatic improvements in efficiency and cost-effectiveness. Such aspects allow for the coupling of antibody polynucleotides, which have elements comprised of a universal sequence, a barcode sequence, and gene or nucleotide targeting sequences, together with individual cells into micron-sized vessels, as well as with beads containing primers which are separately barcoded. The process is performed in such a manner so to allow the primers contributed by the beads work with the primers contributed by the polynucleotide after it is digested with a restriction enzyme, to allow for the amplified DNA from that cell to be tagged for not only its cell identity but also for its sample identity. This allows cells to be multiplexed or pooled prior to encapsulation into the vessels, allowing numerous samples to be run through equipment and workflows that accomplish this encapsulation at the same time.

### CROSS-REFERENCE TO RELATED APPLICATIONS

This application claims the benefit of U.S. Provisional Application No. 63/130,559 filed on Dec. 24, 2020 and entitled “Enhanced Methods And Compositions For The Application Of Single Cell Next Generation Sequencing In Diagnostics And Forensic Science,” and U.S. Provisional Application No. 63/277,529 filed on Nov. 9, 2021 and entitled “Enhanced Methods And Compositions For The Multiplexing Of Single Cell Or Nucleus Next Generation Sequencing Samples For Reducing Costs And Improving Throughput,” the contents of which are incorporated herein by reference in their entirety.

### SEQUENCE LISTING

This application contains a Sequence Listing which has been submitted electronically in ASCII format and is hereby incorporated by reference in its entirety. The ASCII copy, created on Aug. 30, 2022, is named FrudakisSeqListUpdate-ascii and is 63,612 bytes in size.

### TECHNICAL FIELD

The present disclosure generally relates to next generation sequencing (NGS), including single-cell NGS (SCNGS), and more particularly to enhancements to single cell or nucleus next generation sequencing for improving throughput and reducing costs.

### BACKGROUND

Deoxyribonucleic acid (DNA) evidence of a crime scene is almost always derived from a mixture of donors and, as a result, forensic scientists often produce “average” results of the crime scene evidence. For example, if two individuals handled a firearm or other weapon before it was used in a homicide by a third individual, the analyst gets a composite of all three (3) profiles mixed together in what is referred to a “mixture.” Mixtures are often more complex and involve more than just three contributors. When mixture numbers increase and mixture proportions are relatively even, as is often the case, they cannot be resolved. As a result, the evidence from which they came is often uninformative to the case in court.

FIG. 1 shows a part of a typical crime-scene sample, a profile 100 for which it is difficult to resolve contributors. In particular, FIG. 1 reveals four (4) genetic positions 102 from left to right as indicated by the boxes at the top. Considering a position D1 locus indicated as “D1S1856,” one will note five (5) boxes, one box for each allele. Each person has only two alleles at every genetic position (or locus), and therefore, the presence of five (5) alleles indicates that there are at least three (3) contributors to the profile 100 (although this is not even certain). In this case, if profile 100 were taken from a firearm used to commit a murder, how does one resolve the genotypes into individual components, or genotypes corresponding to their discrete human contributors? Put another way, how do the alleles or boxes allocate to each of the three individuals?

To illustrate by example, if there are three contributors, do the 11 and 13 alleles go together for one contributor, with the 15 and 15.3 to another, leaving the 16 as a homozygous third? Or does the 13 pair with 15 with some peak height imbalance, leaving the 11, 16 pairing and a 16 homozygote? Or are there four contributors, with four homozygotes and one heterozygote? Note that the problem is even more difficult for the “D2S1336” locus in FIG. 1. Even further, it becomes overwhelming to consider all the possibilities for, not just this one contributor, but the other two as well.

It is possible to combine them all into a single mixed (e.g., composite) profile and compare suspect standards with this mixed profile, but the statistics of such comparisons are weak due to the uncertainty involved. This weakness creates significant downstream problems when searching for unknown contributors in national databases, such as the Combined DNA Index System (CODIS), which is the United States' national DNA database created and maintained by the Federal Bureau of Investigation (FBI).

To further explain in relation to FIG. 1, when the peaks are of different heights, as is the case at D1S1856, a logical framework can be applied manually to “deconvolute” the contributors one genetic position at a time. In this case, the two highest peaks at D1S1856 (namely, 15, 16) would seem to go together, leaving the other three (3) alleles as representing the remaining two contributors. However, how do these remaining contributors go together? Relative peak heights are generally indicative of contributor ratios but these are similar in height. Also, they are not always indicative of contributor ratios, because minor alleles of the same value can stack on top of each other, and because amplifications that produce the peaks are not always fully predictable due to what is called stochastic variation. Therefore, such a manual method is subject to considerable uncertainty, often producing results of limited value or even nuisance value later downstream.

In response to this problem, statistical geneticists have developed new software tools for Probabilistic Genotyping (PG) that alleviate the burden of manual de-convolution of alleles at each genetic position. These tools can consider more sophisticated metrics in a more standardized way. They are reliable in disentangling simpler mixtures of uneven proportions, but when the mixtures involve too many contributors (or even for simple ones), and/or when the contributors are evenly represented (e.g., 0.3, 0.28, 0.32 in a 3-person mixture), even they are not helpful.

Even when they are helpful, they are extremely difficult to explain in court, which limits their utility. To this day, you can say the words “mixture problem” to any forensic DNA analyst and they will immediately know what you are referring to. The mixture problem affects roughly two-thirds of all forensic samples analyzed in law enforcement crime labs, and, of these, two-thirds more than one-half of the profiles cannot be deconvoluted. They are either processed as less informative mixture profiles or, more often, simply discarded as not interpretable. Each year, a large number of violent crimes go unsolved because the perpetrator was either lucky or smart enough to use an item that had been used by or exposed to others before.

With singe-cell next generation sequencing (SCNGS), a full transcriptome (i.e., the entire collection of RNA molecules) or genome (e.g., epigenome, exome, or full) is determined for each cell of a sample, such that each cell produces its own identity profile or “vector.” Cells are encapsulated in tiny microreactors or droplets, within which the molecular biology takes place in isolation of every other cell, generating work products (e.g., amplified DNA) that is tagged with a unique barcode identifier. While the technology infrastructure exists for applying human identity panels in SCNGS, low throughput and high costs have prevented adoption.

To further explain, a typical vendor offering enables up to either (8) samples processed from cells to NGS library suitable DNA at a time at a cost of over $1,000-$3,000 per sample, depending on the number of samples processed and the vendor(s), and recent “cellplexing” versions of the technology are able to get the pricing down to about $60 per sample, with considerable work. The existing state-of-the-art forensic and diagnostics platforms, which do not provide single cell resolution and therefore do not solve the mixture problem or sensitivity problems, costs only $40 per sample. The further below $40 per sample that SCNGS profiling from forensic evidence can fall, the higher the likelihood of adoption of the technique.

Thus, there is a need in the field of forensic science for SCNGS to be further multiplexable relative to current state-of-the-art, so that profiles can be developed from cells contributed by different donors in sample mixtures. In forensics, it has been identified that one only needs a small sample of cells from each donor as exemplars for determining that donor's identity profile, not each of the donor's sometimes thousands of cells, of various cell types. Data from the entirety of the sample's complexity is not needed and, in fact, can consume valuable bandwidth in multiplexed reactions.

Similarly, in the fields of pre-clinical research, biomarkers are used to track tumor types and subtypes apparent from SCNGS data, such that treatment efficacy can be assessed prior to chemotherapy or after chemotherapy in the event of relapse. Tumors exist in complex microenvironments incorporating various normal cell types, and various clones or subtypes of tumor cells. The vast majority of SCNGS technology is designed for deep assessments of samples, such that large numbers of cells are sequenced, revealing the entire subtype complexity. In some situations, such as in diagnostic situations, or Drug Sensitivity Screening (DSS) screening situations, it has been identified that data from the entirety of the sample's complexity is not needed and, in fact, can consume valuable bandwidth in multiplexed reactions.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts.

FIG. 1 is an example of a deoxyribonucleic acid (DNA) profile for which it may be difficult to resolve contributors;

FIG. 2A is an illustration depicting a processing step associated with cellular isolation and extension on beads for Single Cell Next Generation Sequencing (SCNGS);

FIG. 2B is an illustration of a bead coupled to two oligonucleotide sequences;

FIG. 2C is an illustration of amplification products which are produced for each cell in isolation to every other cell, where each amplification product contains a unique cell and droplet specific barcode sequence;

FIG. 2D is an illustration showing that, after a library prep stage, sequences produced from each cell may be identified by the unique barcode, so the products from all cells of a sample may be pooled;

FIG. 2E is an illustration showing that state-of-the-art SCNGS multiplexing may allow for the cells to be tagged with a barcode through fusion of lipid nanodroplets containing this barcode linked to a universal primer;

FIG. 2F is an illustration showing that, as a result of the processing, sample multiplexing may be limited to only a few samples per channel;

FIG. 3 is an illustration of a workflow showing sequential steps of state-of-the-art SCNGS (without the dashed box), which may be enhanced with use of a module for enhanced sample tagging according to the present disclosure and inserted prior to the library preparation process of the SCNGS process;

FIG. 4 is a flowchart for describing an enhanced method of single cell or nucleus next generation sequencing according to some implementations of the present disclosure;

FIGS. 5A through 5M are example illustrative representations of processing steps associated with the method described in relation to FIG. 4 according to some implementations of the present disclosure;

FIGS. 6A, 6B, and 6C are plots of a function n*(1/x³) that show similar forms taken for various values of n as that obtained with a program written for optimization of statistical dilution steps;

FIG. 7A is a grid of an index system for the selection of random barcodes of polynucleotide concatenates which may be used to tag the sample identities;

FIG. 7B is a table of 5′ primers represented in the polynucleotide concatenates, for demonstrating the creation of polynucleotide concatenates per the example in the List Of Polynucleotides at the end of the specification;

FIG. 7C is a table of 3′ primers represented on vendor-supplied beads, for demonstrating the creation of polynucleotide concatentates per the example in the List Of Polynucleotides at the end of the specification;

FIG. 7D is a table which shows each primer being joined with a universal and barcode sequence, for demonstrating the creation of polynucleotide concatentates per the example in the List Of Polynucleotides at the end of the specification;

FIG. 8A is an illustration of a microfluidic device for enhanced single cell or nucleus next generation sequencing according to some implementations of the present disclosure, with use of modules which include a preparation module, a digestion module, and a pre-amp module;

FIG. 8B is a flowchart for describing a method of a three-step microfluidic process according to some implementations of the present disclosure, associated with the microfluidic device of FIG. 8A;

FIG. 9 is a flowchart for describing an enhanced method of single cell or nucleus next generation sequencing according to some implementations of the disclosure;

FIGS. 10A through 10G are example illustrative representations of processing steps associated with the method described in relation to FIG. 9; and

FIGS. 10H and 10I are example illustrative representations of alternative processing steps associated with use of alternative polynuclotides.

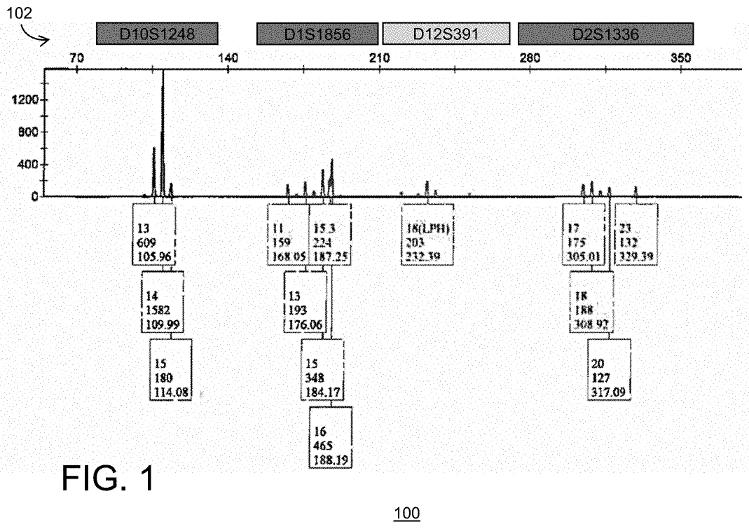

### DESCRIPTION OF EXAMPLE EMBODIMENTS

### Overview

What are described herein are enhancements to single cell or nucleus next generation sequencing for significantly improving throughput and reducing costs.

### EXAMPLE EMBODIMENTS

The present inventive work relates to improved cost-effectiveness and functionality of human identity casework and diagnostics. Inventive aspects of the present disclosure may effect a disruptive transformation in these markets by enabling for the first time, cost-effective targeted gene profiles from individual cells part of sample mixtures. What is enabled is a more economical and efficient version of “horizontal” or gene-targeted Single Cell Next Generation Sequencing (SCNGS), such that markets, heretofore resistant to adoption, are opened up. SCNGS may sometimes be referred to as “low throughput” NGS, and NGS is sometimes referred to as Massively Paralleled Sequencing.

Notably, the present inventive work may help, once and for all, establish SCNGS as a means by which the “mixture problem” which has plagued the forensic genetics and human identity market since its inception in the late 90's can be solved in a cost-effective manner, likely to be adopted. In so doing, the present inventive work could help bring about an inflection point and establish a new gold standard or state-of-the-art in forensics and diagnostics. Just as notably, the inventive aspects disclosed herein may help establish a framework for translating recent SCNGS advances to the areas of diagnostics, which require low per-sample costs.

Although the present disclosure may focus on the forensic sciences in many instances, the mixture and sensitivity problems apply to clinical diagnostics as well, where, instead of losing information from cells corresponding to minor profile contributors, what is lost is information from cells corresponding to minor tumor subclones, or, in the case of cell culture authentication, for example, from minor cell culture contaminants.

A multi-step method and associated compositions for multiplexing different samples into single runs at significantly greater scale than heretofore possible, for use with typical vendor equipment and reagent platforms, are described.

According to some aspects of the present disclosure, what is involved may be or include a kit, constituting a set of consumables (e.g., conjugated multiplex primer solutions, plastics) and reagents (e.g., enzymes, deoxynucleotide triphosphates “dNTPs”, buffers, etc.) that seamlessly integrate with and elegantly transform recently-introduced SCNGS platforms for forensic genetics casework. The advance provided is in improving an established technology of amplifying deoxyribonucleic acid (DNA) from isolated cells such that it becomes cost-effective and efficient enough for application within the fields of forensic science and clinical diagnostics. According to some aspects, the present inventive work may enable SCNGS platforms to produce multiplex Short Tandem Repeat (STR) and Single Nucleotide Polymorphism (SNP) human identity (and related forensic phenotype) profiles from single cells over a large number of samples, such that the cost per sample and number of samples that can be processed in a day or week becomes competitive with the current, non-single cell state-of-the-art. In other aspects, the present inventive work may enable SCNGS platforms to produce sequence reads at significantly lower costs per sample for certain diagnostic target gene Research and Development (R&D) applications.

According to some aspects, the present inventive work may involve methods for the construction of conjugated multiplex primer sets and molecular biology workflow for seamless integration into existing SCNGS protocols to enable cells from different forensic or diagnostic samples to be multiplexed at high multiplicity into single runs through an SCNGS workflow. Instead of a single lane or channel of a SCNGS cartridge being devoted to a single sample as in standard SCNGS, or to 8 samples as with multiplexed SCNGS offered by some vendors, with the present invention, this lane can be devoted to dozens, hundreds or even thousands of samples, depending on the number of target genes and the desired depth per sample.

Single Cell (SC) systems to date have targeted the R&D market, and therefore have been designed as “vertical” platforms, allowing deep analysis of relatively few samples (such as the full genomes of many thousands of cells for a couple or few different samples). Because the goal of forensic and diagnostic applications of DNA sequencing are more “horizontal,” requiring relatively shallow data on a representative and small sampling of a sample, much of the capacity of present SC systems is not needed.

To overcome the above, SCNGS vendors have developed methods for tagging cells corresponding to discrete samples, so that cells of different samples can be combined prior to the expensive step of encapsulating cells into microdroplets, dramatically reducing the cost per sample. To do this, the vendors have used lipid vesicles containing oligonucleotide tags, where each tag is allocated to each sample of cells. The vesicles fuse with the cell membrane of these cells, releasing the tag oligos inside. Other gene targeting 5′ and 3′ primers, or just 5′ or just 3′ primers are provided on separate beads that are encapsulated with the cell, each bead containing a unique barcode that identifies the products from a particular cell. The other primers can be provided in bulk. The tag oligos become incorporated into amplification products from the cell by virtue of a universal sequence at the 3′ end, and tag these products by virtue of a barcode sequence at the 5′ end.

The problem with this method of multiplexing is that each of the cells in the sample consume reagents needed to produce the amplification products. This consumption utilizes channel bandwidth in subsequent cartridge and surface chemistry platforms needed to produce the SCNGS reads. When each cell of a sample occupies space in these platforms, it limits the degree of multiplexing that can be accomplished due to the platform's limited space.

According to some aspects of the present disclosure, a mechanism is provided by which amplification of a cell in SCNGS is accomplished for only select cells of a sample. This selective processing reduces the complexity of each donor's contribution to the reads, enabling a horizontal analysis, or skimming of the samples, needed in some R&D and routine diagnostics applications and virtually all forensic science applications. Such processing may bring the cost per sample down to levels suitable for mass adoption.

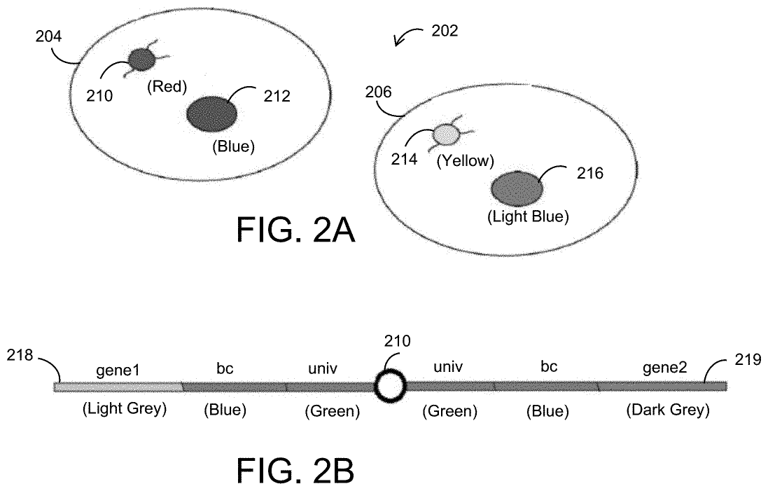

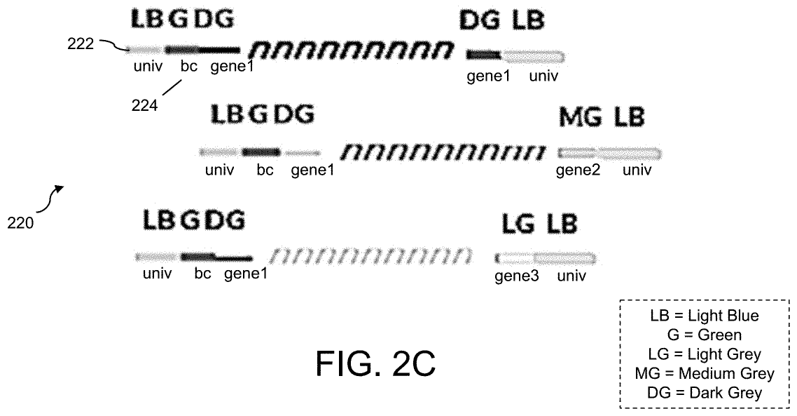

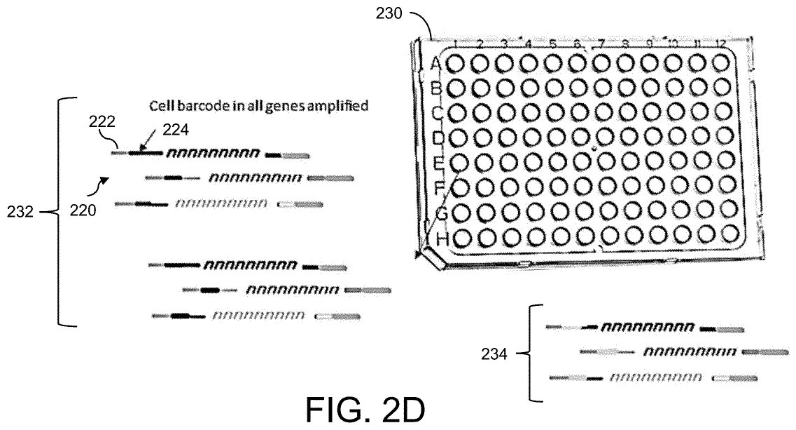

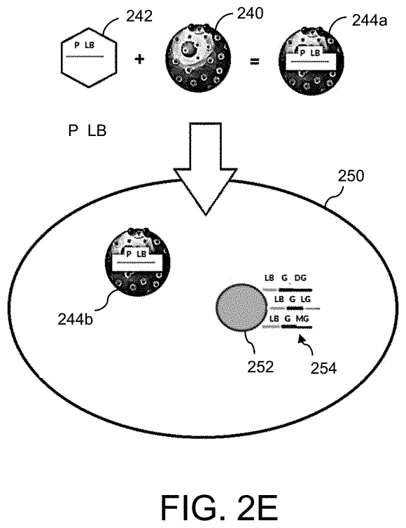

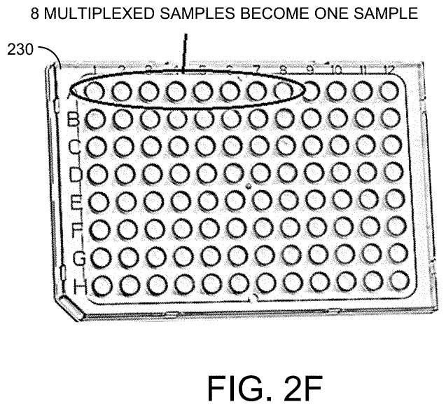

To better illustrate in relation to the figures, FIG. 2A is an example illustrative representation of a processing step associated with SCNGS cellular isolation and extension on beads. In FIG. 2A, two nanoreactors are shown; in particular, a nanoreactor 204 and a nanoreactor 206. Each one of nanoreactors 204 and 206 include a bead and a cell; in particular, nanoreactor 204 includes a bead 210 (“red”) and a cell 212 (“blue”), and nanoreactor 206 includes a bead 214 (“yellow”) and a cell 216 (“light blue”).

With targeted gene SCNGS of the background/related art, the basic idea is to create a large number of micrometer or nanometer sized spaces, which may be termed microvesicles or “reactors”; such spaces may be microdroplets or nanodroplets on a solid surface or substrate, or physical microchambers, or microwells or nanowells created via electrokinetic gating (all of the foregoing which may be referred to hereinafter as microspaces or nanospaces, or microreactors or nanoreactors). Into these spaces are introduced an individual cell or nucleus along with a bead linked to barcoded primers. Subsequent thermal cycling steps enable extension of the primers along the target gene region, followed by amplification of the target loci for subsequent reading with next generation sequencing (NGS).

FIG. 2B is an example illustrative representation of bead 210 which is physically coupled to two oligonucleotide sequences, namely, an oligonucleotide sequence 218 (left) and an oligonucleotide sequence 219 (right). Each of these oligonucleotide sequences 218 and 219 includes a universal primer binding site (or universal sequence) (“univ”—green), a barcode sequence (“bc”—blue), and a sequence-specific primer (or gene sequence) (“gene1”—light grey for oligonucleotide sequence 218; and “gene2”—dark grey for oligonucleotide sequence 219). Here, each oligonucleotide sequence may be referred to or abbreviated in the form of “univ-bc-gene.” In some implementations, the universal primer binding site may be a combination of universal primer binding sites. The universal primer binding site may be common to all of the oligos generated but common to each bead, whereas the barcode sequence may vary from bead to bead. In some applications (e.g., transcriptome analysis), the sequence-specific primer is the same for each primer (e.g., poly dT). In other applications (e.g., targeted gene sequencing), a plurality of gene sequences may used. Using a microfluidics machine provided by a vendor, the user may inject a single bead containing these oligonucleotide sequences and a single cell into a microreactor. As each of the primers amplify, the universal and barcode sequences are integrated into the amplification products of the amplification.

Upon lysis of the cell, and introduction of reagents for Polymerase Chain Reaction (PCR), the oligos bind to genetic material released by the cell or nucleus whereupon the oligo is extended in an extension step. The reagents for PCR may be or include, for example, dNTPs and, in some cases, reverse transcriptase, and thermostable or other DNA polymerase enzyme. For applications targeting ribonucleic acid (RNA) transcripts, complimentary (cDNA) is first created for each messenger RNA (mRNA). Once the bead is dissolved enzymatically or photochemically, the oligos are released, find the RNA, and convert it to cDNA. For applications targeting DNA loci, this cDNA step is not necessary; extension and amplification take place directly on the released genomic DNA.

Because each oligo attached to the bead contains a common and unique, bead-specific barcode incorporated into its sequence, all genetic sequences extended and subsequently amplified from it are identifiable as having been originally captured and extended from the oligos provided by a particular bead. As the bead was encapsulated with a single cell or nucleus in its nanoreactor, all amplification products generated inside the microvesicle may be attributable to that single cell.

For vendors targeting DNA instead of RNA, the oligos attached to the beads contain a universal sequence, a barcode sequence, and a 3′ common sequence. Here, each oligonucleotide sequence may be referred to or abbreviated in the form of “univ-bc-comm.” A panel of primers may be generated to target specific loci, where each primer contains the common sequence at the 5′ end, which thereby facilitates incorporation of the barcodes into the amplification products. The 3′ primers will often contain another, different universal primer sequence at their ends. Here, the result is that the amplification products from mRNA (or cDNA) or genomic DNA each contain a unique, cell and droplet specific barcode sequence.

FIG. 2C is an example illustrative representation of a plurality of amplification products 220 which may be produced for each cell, in isolation from every other cell, as described. Each amplification product may contain a unique cell and droplet specific barcode sequence. For example, the plurality of amplification products 220 include an amplification product 222 having a barcode 224 (“bc”, indicated here in green “G”) which uniquely identifies a single cell. Thus, the plurality of amplification products 220 having the unique barcode can be attributed to a single cell. Also in FIG. 2C, the universal primer (“univ”) is indicated as LB for light blue and the barcode sequence (“bc”) is indicated as G for green. In addition, the gene1-specific sequence (“gene1”) is indicated as DG for dark grey, the gene2-specific sequence (“gene2”) is indicated as MG for medium grey, and the gene3-specific sequence (“gene3”) is indicated as LG for light grey, which are associated with the oligonucleotides used as primers to amplify from each samples cells. If the set of gene specific primer regions recognizes 24 DNA targets, then 24 different sequences will be produced for each cell. If it recognizes 16,000 (as with mRNAs), then 16,000 different sequences will be produced for each cell.

After this processing, the amplification products may be prepared for NGS, by amplifying each with a unique pair of tagged universal primers in a grid format. Using the grid format, samples of each column receive a 5′ universal primer with a unique tag, and samples of each row receive a 3′ primer that matches the second universal site linked to a unique tag. Here, the sample can be identified by the pair of tags incorporated into the resulting amplification products.

The barcodes only resolve the molecule (one barcode) and the cells (another barcode) within the sample, and do not identify the sample. Therefore, each sample must be processed in isolation of each other. This processing takes several hours and consumes many expensive cartridges and reagent kits. Here, the run products must be kept track of manually for later next generation sequencing, which is error prone. Costs are roughly $5500 per sample all the way through NGS, which is far too expensive for routine diagnostics, R&D, or forensic science applications which targets costs of $40 or less.

After library preparation, sequences produced from each cell are identified by the unique barcode, and therefore the amplification products from all cells of a sample can be pooled together. Different samples of cells are then separated into different channels, however, because there is no way at this stage to identify the sample from which the cell and sequence was derived.

FIG. 2D is an example illustrative representation of a microtiter plate 230 which may be utilized for channel separation and processing of different samples of cells, including, for example illustration, a plurality of amplification products 232 of a first sample of cells and a plurality of amplification products 234 of a second sample of cells.

Using channel separation, each of the expensive cartridges and reagent kits can only handle eight (8) samples at a time. There is a new alternative method available that allows multiplexing of eight (8) samples per channel, but this only gets costs down to $687 per sample. A primary driver of costs is the expense of the reagents, labor and consumables for the single cell isolation portion of this workflow.

Thus, the above-described process may be multiplexed eight-fold with use of state-of-the-art SCNGS multiplexing. This multiplexing may be performed by adding lipid vesicles with tag primers to the cells of each sample. The vesicles fuse with the cells, and the tag primers are internalized by the cells. The tag primer may contain a sample-specific barcode and a 3′ end that matches a terminal sequence on the beads, allowing them to be incorporated into the amplification products produced by the bead primers.

FIG. 2E is an example illustrative representation depicting the state-of-the-art SCNGS multiplexing, where a cell 240 may be tagged with a barcode to result in a cell 244a, through fusion of a lipid nanodroplet 242 containing the barcode linked to a universal primer. Once integrated into a microreactor 250 (e.g., a microdroplet) with the cell (now, a cell 244b) along with a bead 252 coupled to a plurality of oligonucleotides 254 for targeting the target set of loci, and a companion primer set (not shown), each amplification product will contain the barcode through which the sample of origin can be attributed. The downslide of this approach for horizontal or ultra-low throughput SCNGS is that all of the cells receive the barcode and/or consume amplification reagents, contributing amplification product which consumes the cartridge bandwidth.

Cartridges containing, for example, eight (8) sample wells of a microtiter plate may be used to process eight (8) samples at a time, at costs around several hundred dollars per sample. Multiplexing of samples to reduce per-sample-costs in this processing step may be achieved by using beads as described above which contain half of the primers targeting the specific regions of interest (e.g., bead 252 with the plurality of oligonucleotides 254 of FIG. 2E). For example, this sequence may be provided in the form of an oligonucleotide containing a universal primer binding site (or universal sequence) (“univ”), a barcode sequence (“bc”) which is bead and cell-specific, and a gene-specific primer (or gene sequence). In some implementations, the barcode region may also include other barcodes as well, for example, those referred to as unique molecular identifiers (UMIs). The other half of the primers needed to amplify the sequences of interest may be provided in bulk across all samples, and contain an additional universal primer binding region (not shown in FIG. 2E). A third primer containing a unique tag may also be provided to each sample. This third primer may be incorporated into lipid nanodroplets or vesicles (e.g., lipid nanodroplet 244a), which fuse with the cells (e.g., cell 244b) of the sample or an antibody which binds to an epitope expressed on the membrane of cells of the sample. This third primer may contain sequences that hybridize to the second universal region at its 3′ end. As the cell (e.g., cell 244b) is encapsulated into microreactor 250 with the bead-linked first set of primers (e.g., bead 252 with the plurality of oligonucleotides 254) and the second set of bulk primers (e.g., the 3′ primers, not shown in FIG. 2E), the third sample tag primer is also incorporated into microreactor 250 and becomes integrated into the amplification products of cell 244b by virtue of annealing at the second universal region.

FIG. 2F is an example illustrative representation of microtiter plate 230 indicating that eight (8) multiplexed samples effectively become one sample with use of the state-of-the-art SCNGS multiplexing. Kits sold by some vendors enable the multiplexing of samples prior to encapsulation in the above-described manner, up to eight-fold, reducing costs from several thousand dollars per sample to several hundred dollars per sample. If each sample of cells is allocated to a single well of a microtiter plate, eight (8) wells or samples may be combined after this step to occupy one “lane” or “channel” in the SCNGS system, getting the costs down to about $650 per sample all the way through NGS.

With use of the above-described sample multiplexing processing, the depth of each sample is not significantly diminished and the data produced are as rich as that produced from unmultiplexed sample processing. For example, the same number of reads per target locus in the same number of cells (e.g., thousands) is achieved. As is apparent, the above-described process consumes valuable channel bandwidth.

For forensic and certain diagnostics-targeted SCNGS applications, only a dozen or few dozen cells worth of data from each sample are needed for identification. In certain R&D applications, it is desired to track only cells of a certain type, such as tumor cells instead of normal cells. The remaining thousands of cells for which data are not needed consume reagents and cartridge bandwidth (e.g., well space or two-dimensional surface area), which is wasteful in these applications. As is apparent, conventional processes impose limitations on the number of samples that may be multiplexed and corresponding limitations on the cost-per-sample reduction that can be achieved.

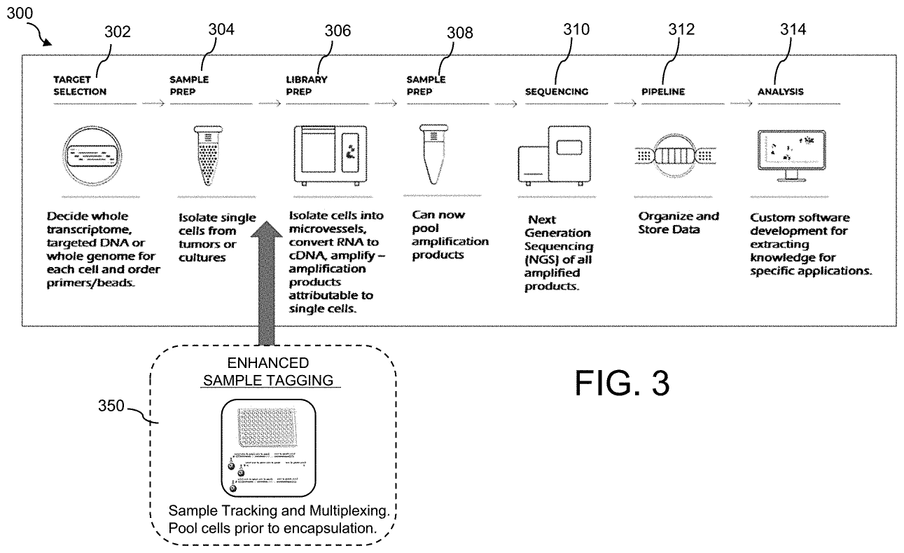

FIG. 3 is a workflow 300 which illustrates the basic sequential steps of current, state-of-the-art SCNGS (without the dashed box). Workflow 300 of FIG. 3 (without the dashed box) is from the website of the main vendor that provides equipment and reagents for a method of SCNGS DNA sequencing, 10X Genomics Inc. of Pleasanton, Calif., U.S.A. As illustrated, workflow 300 is comprised of a target selection process 302, a sample preparation process 304, a library preparation process 306, a sample preparation process 308, a sequencing process 310, a pipeline process 312, and an analysis process 314. Target selection process 302 may involve deciding whole transcriptome, targeted DNA, or whole genome for each cell, and ordering or primers/beads. Sample preparation process 304 may involve isolating single cells from, for example, tumors or cultures. Library preparation process 306 may involve isolating cells into microvessles, converting RNA to cDNA, and amplifying, where the amplification products may be attributable to single cells. Sample preparation process 308 may involve pooling the amplification products together. Sequencing process 310 may involve next generation sequencing of all of the amplified products. Pipeline process 312 may involve organizing and storing data. Analysis process 314 may involve extracting knowledge from the data for specific applications based on custom software development.

It is understood that workflow 300 (without the dashed box) produces data at about $5,500 per sample and a throughput of eight (8) samples per week. The throughput (e.g., only 8 samples per week, depending on labor investment) and cost (e.g., up to $5,500 per sample) are problems that are debilitating for forensic science and diagnostics use, which may target costs at less than $40 per sample and a throughput of hundreds of samples per week for SCNGS to be adopted.

All of this background is known to those familiar with the current state-of-the-art and SCNGS datasets which are published monthly. Conventional “sample” multiplexing attempts to resolve some of the above-indicated problems. Conventional sample multiplexing involves the insertion of a conventional “sample tagging” step, where cells are allocated to microreactors, prior to performing library preparation process 306. Use of the conventional sample tagging step provides modest cost reductions down to hundreds of dollars per sample (e.g., about $500) and modest throughput gains up to 64 samples per week.

Even with sample multiplexing, SCNGS is not presently employed for forensics and routine diagnostics. The reason is that existing protocols and platforms provide for price-per-sample reductions to about $100, whereas competing bulk technologies that are currently used perform at prices of about $40 per sample or less. This particular problem may be referred to herein as the “SCNGS cost problem.” Further, breaking a set of samples into, for example, 8 sets of 8 multiplexed samples, imposes significantly more work in terms of pipetting and tracking than would be the case if the set of 64 samples were processed simultaneously. Laboratory technicians, daunted by the challenge, only attempt such multiplexing if they are highly-experienced and ambitious, which results in non-adoption of the processing. This other particular problem may be referred to herein as the “SCNGS throughput problem.”

According to at least some implementations, an enhanced sample multiplexing process involving enhanced sample tagging may be inserted prior to library preparation process 306 of FIG. 3. In some implementations, the enhanced process is performed using a superior molecular biology approach that allows for a “skimming” of the sample of cells so that channel bandwidth is preserved for analysis of far larger numbers of samples simultaneously.

Thus, in some implementations, the present invention may constitute a series of steps in the form of a module 350 that fits into and is inserted into the workflow 300 as indicated in FIG. 3 (see the arrow which inserts the module 350 into workflow 300). The series of steps of module 350 may be performed prior to loading samples into the cartridge channels. In some implementations, with use of module 350 in the workflow 300, the workflow process may be adjusted to produce data at about $2-$8 per sample with a throughput that is greater than 3,000 samples per week.

In at least some implementations, the inventive processes may overcome both the SCNGS cost and throughput problems at the same time. Preferably, the above may be achieved with use of true sample multiplexing, at a meaningful scale, using an innovative molecular biology approach that is seamlessly-integrated into existing transcriptome or targeted DNA SCNGS workflows.

In some implementations, the inventive processes may achieve the above by allowing a multiplexing of samples prior to injection into microreactors, such that larger numbers of samples (e.g., 96, 384, or more) may be processed at a time. This may be accomplished by attaching the second set of primers to an agent, such as an antibody (e.g., proteoglycan or glycoprotein), such that only microreactors containing particular cells capable of binding to the agent receive both of the primers for each target locus, produce amplified DNA, and consume reagents and single-cell as well as NGS cartridge bandwidth.

The selective targeting of only particular cells of each sample may be achieved by using agents that recognize and bind to cell or nuclear membrane proteins and/or sugars that express the relevant epitopes or cellular targets. For example, with a forensics sample containing a mixture of tissue and cell types, epithelial cells may be targeted for selection. As another example, with a forensics sample containing a mixture of blood cells, CD34 positive white blood cells may be targeted for selection. As yet another example, a cell culture sample used in drug sensitivity screening or other diagnosis, one could target cells expressing aberrant “glycomes” or cell-surface sugar profiles using proteoglycans or glycoproteins identified from other systematic screening processes, thereby eliminating normal cells from the analysis, and restricting the use of reagents and consumables bandwidth to tumor cells.

In at least some implementations, each cell may be essentially tagged with its “sample” membership (e.g., in a unique well of a microtiter plate), where a subset of the cells in each one of the samples is targeted. The targeting may be performed in order to maximize the efficiency with which the reagents and cartridge bandwidths are utilized, allowing for greater “stacking” of samples per cartridge. For example, if 384 samples are stacked into each sample well of the cartridges, the non-multiplexed $1,000 per sample cost of the single cell portion of the workflow may be reduced to under $1 per sample. Since bulk analysis protocols that lack single-cell resolution cost about $40 per sample, the motivation for adoption becomes not only that of gaining the power of single-cell resolution for solving the mixture and sensitivity problems, but of cost savings. Advantageously, the present inventive work has therefore opened up the forensic science market to SCNGS by alleviating the primary impediments to its adoption.

Accordingly, the present inventive work may overcome both SCNGS cost and throughput problems at the same time with use of an innovative molecular biology approach and workflow that can seamlessly integrate into existing transcriptome or targeted DNA SCNGS workflows.

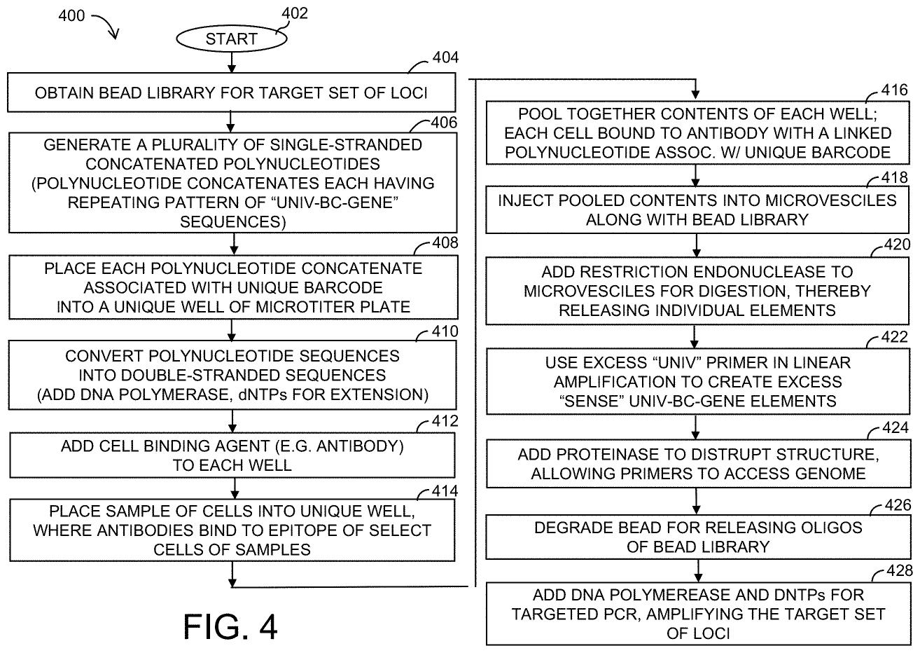

FIG. 4 is a flowchart 400 for describing an enhanced method of single cell or nucleus next generation sequencing according to some implementations of the present disclosure. In an illustrative example, the Combined DNA Index System (CODIS) STRs are selected as the target loci in a targeted SCNGS application. In the illustrative example using CODIS STRs, twenty-four (24) CODIS STR loci may be targeted. Although the illustrative example pertaining to CODIS STRs is provided, it is understood that the process may be applied to other applications, including diagnostics, basic and applied R&D, and at the level of transcriptomics, epigenomics, or proteomics, etc. Note that the method described here may or may not utilize related or similar steps or techniques as described in relation to the above-described techniques. In at least some implementations, multiplexing may be enabled prior to bead or cell injection with as described in the following process.

Beginning at a start block 402 of FIG. 4, a bead library for a targeted set of loci may be obtained or identified (step 404 of FIG. 4). Here, in some implementations, each bead may be linked to a plurality of oligonucleotides for targeting the entire set of loci. In some implementations, each oligonucleotide of a bead may include (e.g., in sequential order) a universal sequence (“univ”), a barcode (“univ”) that is uniquely associated with the bead, and a gene sequence (“gene”) which represents one of a pair of primers (e.g., ‘3 primer) of a primer pair set for amplifying the targeted loci. In some implementations, each oligonucleotide may include one or more universal sequences and/or one or more barcodes in the sequence. In some implementations, each oligonucleotide may include the barcode of the specific oligo linked to the bead. The number “N” of oligonucleotides may correspond to the number of targeted set of loci or genes. For example, each bead may be coated with N oligonucleotides, where N may be in the tens, hundreds, thousands, hundreds of thousands, millions, etc., per bead. In the illustrative example, the targeted set of loci may be the targeted set of CODIS STR loci (e.g., where each bead has 24 different oligonucleotides). In some implementations, the bead library which includes the beads and oligonucleotides (e.g., those described in the background/related art) may be obtained from any one of a number of different vendors, including current state-of-the-art SCNGS vendors, and may be referred to herein as the “vendor” bead library.

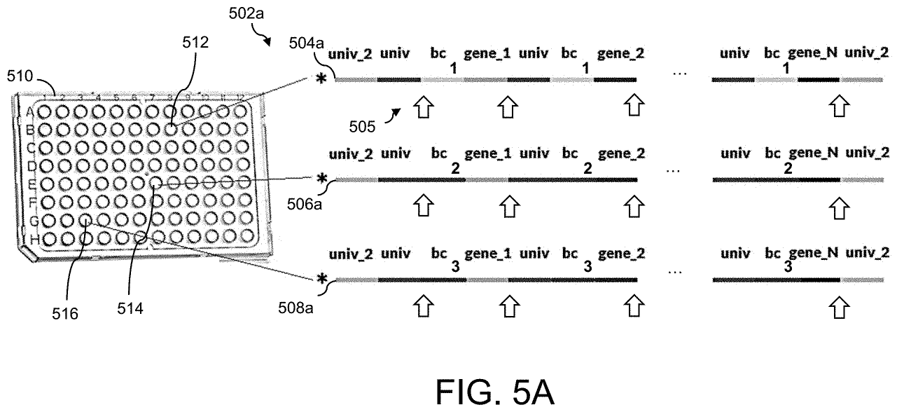

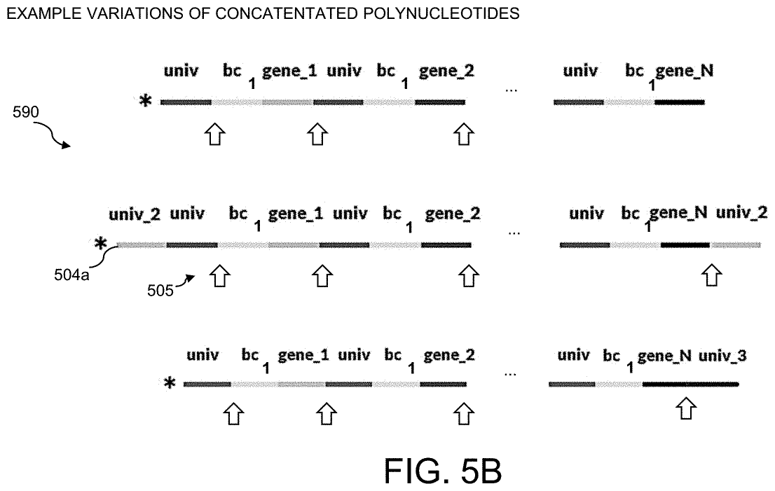

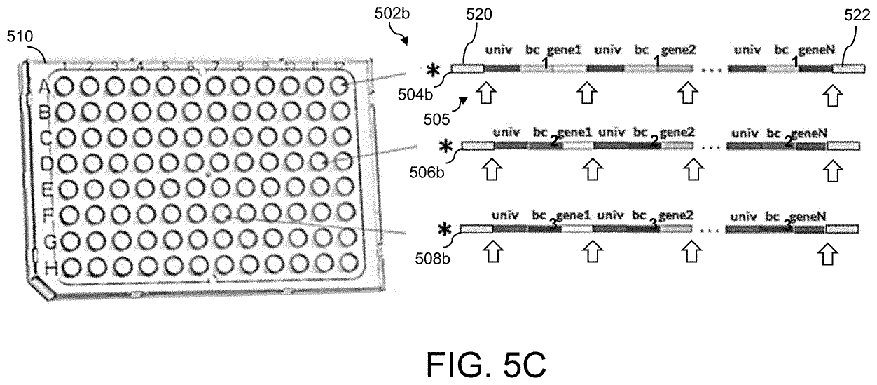

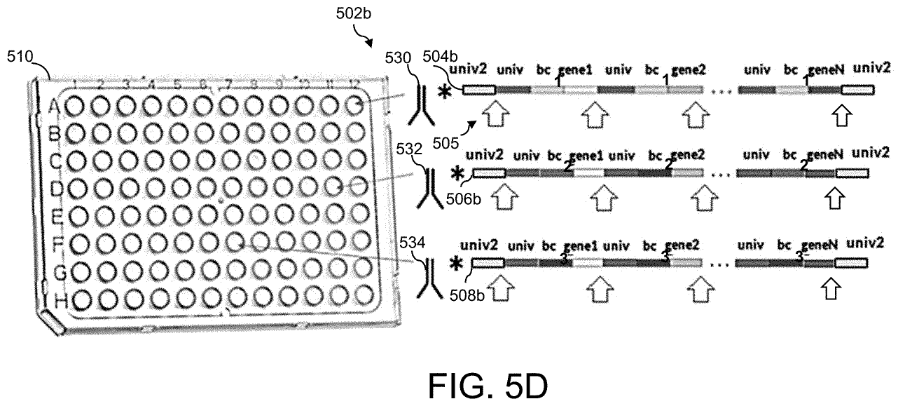

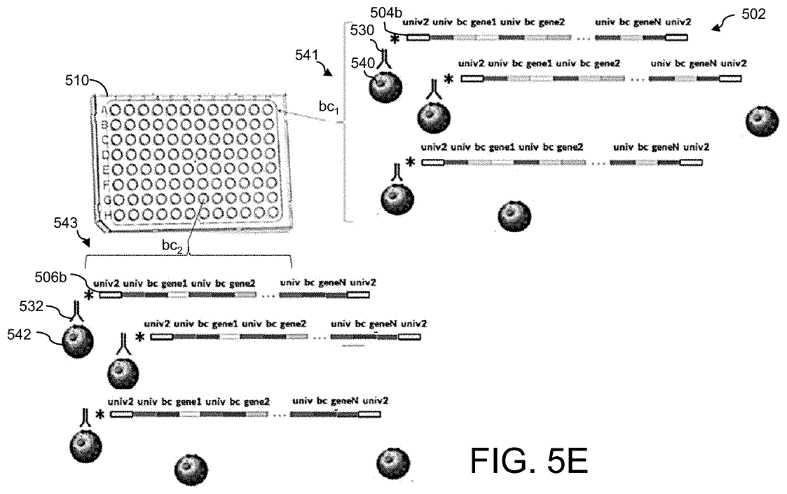

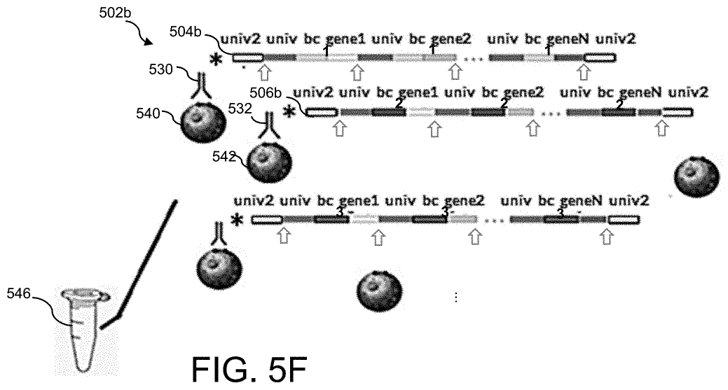

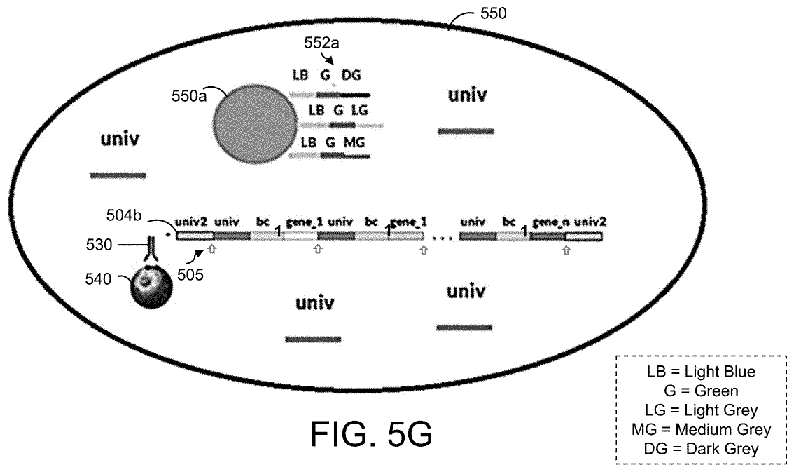

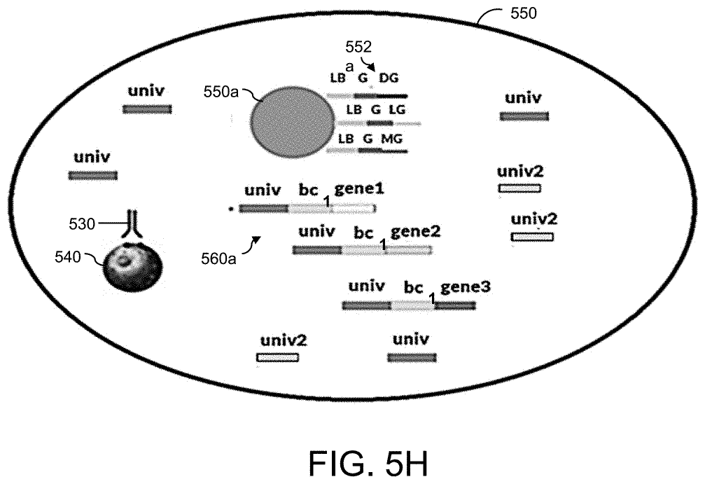

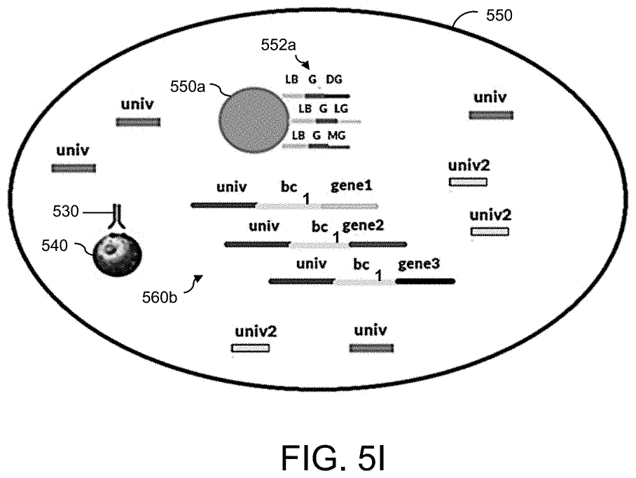

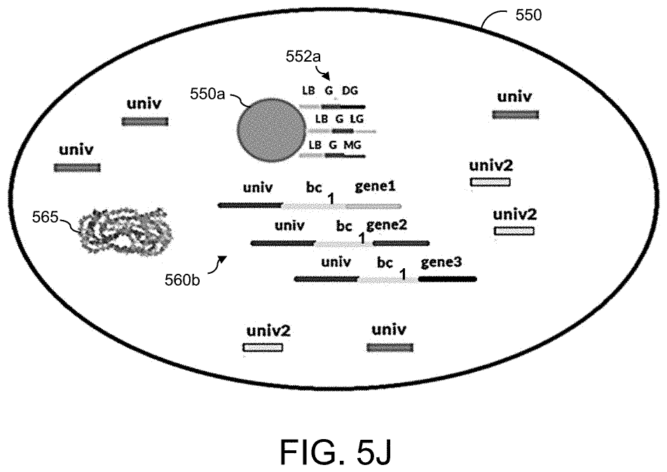

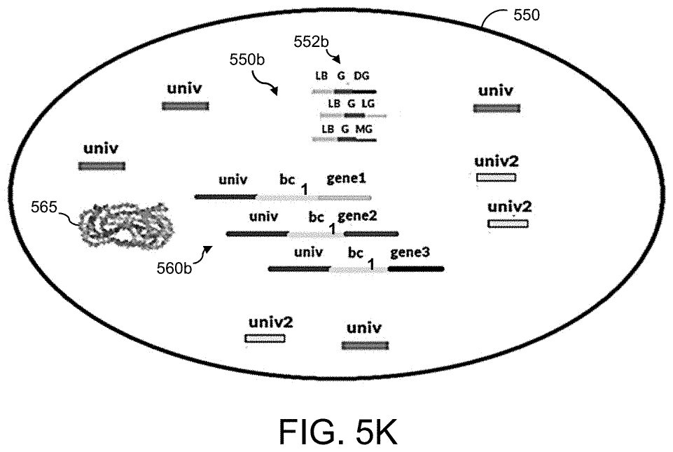

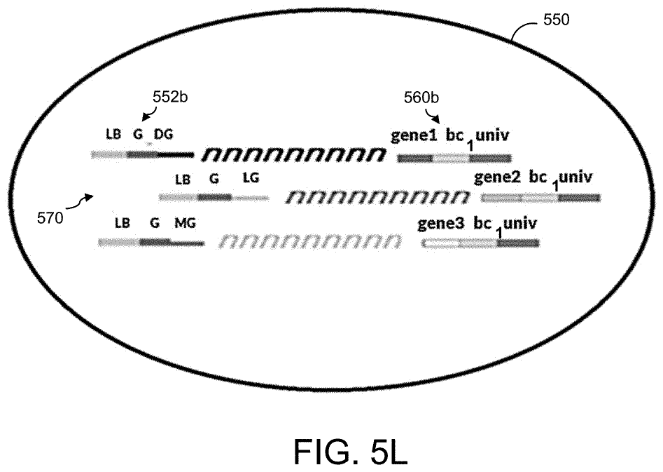

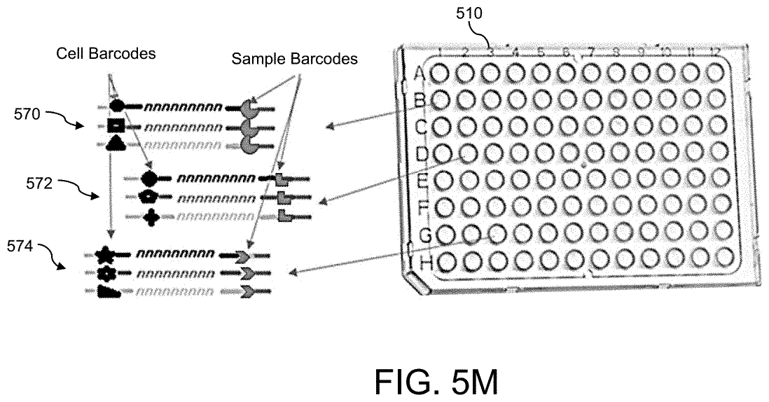

Next, a plurality of single-stranded concatenated polynucleotides may be synthesized or generated (step 406 of FIG. 4). In FIG. 5A, what is shown is an example illustrative representation of a plurality of single-stranded concatenated polynucleotides 502a (or “polynucleotide concatenates”), which include a single-stranded concatenated polynucleotide 504a, a single-stranded concatenated polynucleotide 506a, and a single-stranded concatenated polynucleotide 508a.

As illustrated in FIG. 5A, each single-stranded concatenated polynucleotide may be comprised of a repeating pattern of “univ-bc-gene” sequences, represented as (e.g., in sequential or consecutive order) univ-bc-gene1, univ-bc-gene2, . . . , to univ-bc-geneN, where N refers to the number of targeted loci or genes. Here, “univ” may represent a universal sequence (“univ”) of one or more universal sequences, “bc” may represent one or more barcodes, and “gene” may represent the corresponding one of the pair of primers (e.g., ‘5 primer) of the primer pair set associated with the bead library for amplifying the targeted loci (e.g., gene1=primer for gene1, gene2=primer for gene 2, . . . , geneN=primer for gene N).

In some implementations, each universal sequence (“univ”) may be 10, 15, 20 or 30 or more nucleotides in length and may be synthesized using any suitable state-of-the-art approach. In some implementations, the polynucleotide concatenates may have additional universal primer binding sites (e.g., other than “univ”) as needed, for example, to facilitate subsequent processing steps (e.g., as described later below). In some implementations, each gene sequence (“gene”) may have a size that is selected based on a desired melting temperature Tₘ for amplification reactions.

For example, FIG. 5A shows the plurality of single-stranded concatenated polynucleotides 502a to each include additional universal primer binding sites (e.g., “univ_2”) at their 5′ and 3′ ends. To further illustrate, FIG. 5B are some example variations of single-stranded concatenated polynucleotides 590 of the present disclosure, which include the single-stranded concatenated polynucleotide 504a of FIG. 5A, as well as a single-stranded concatenated polynucleotide having an additional universal primer binding site (e.g., “univ_3”) at its ‘3 end (e.g., bottom of FIG. 5B), and even a single-stranded concatenated polynucleotide not having any additional universal primer binding sites (e.g., top of FIG. 5B).

Also as mentioned above, each single-stranded concatenated polynucleotide may be uniquely associated with a barcode (“bc”) (e.g., which is repeated in each repeating pattern of the polynucleotide concatenate). With reference back to FIG. 5A, single-stranded concatenated polynucleotide 504a may be uniquely identified with a first barcode (“bc1”), single-stranded concatenated polynucleotide 506a may be uniquely identified with a second barcode (“bc2”), and single-stranded concatenated polynucleotide 508a may be uniquely identified with a third barcode (“bc3”), etc. Each barcode may be any suitable sequence, such as a random sequence(s) of N nucleotides, where N may be 10, 15, 20, etc. As is apparent, in the repeating pattern of universal-barcode-gene sequences, each of a dozen or dozen(s) of the gene sequences may be distinct but the barcode sequences are the same. In some implementations, each barcode may optionally include a region that is unique to the oligonucleotide, referred to as a Unique Molecular Identifier (UMI) in the background/related art.

To illustrate the above by example, for a targeted set of loci where N=2, a bead may be associated with oligos including univ-bc-gene1 and univ-bc-gene2 (which provide the 5′ primer sequence for gene1 and gene2), and a single-stranded concatenated polynucleotide may be comprised of univ-bc-gene1+univ-bc-gene2 (which provide the 3′ primer sequence for gene1 and gene2).

In the illustrative example associated with the targeted set of CODIS STR loci, where the twenty-four (24) primers represented in the bead library are 5′ primers for each of 24 different target loci, there may be 24 “univ-bc-gene” sequences that are strung in the polynucleotide concatenate. Each single-stranded concatenated polynucleotide may be comprised of the repeating pattern of “univ-bc-gene” sequences, one (1) common “univ” sequence, one (1) common “bc” sequence, and the 24 different “gene” sequences representing the 3′ primers for the 24 different target loci. In alternative implementations of the above, the 5′ and 3′ primers may be reversed, where the beads with the oligos provide the 3′ primers and the single-stranded concatenated polynucleotides provide the 5′ primers.

For a gene set of twenty-four (24) CODIS STR loci, the length of each single-stranded concatenated polynucleotide may be about three-thousand (3,000) nucleotides long; synthesis of long polynucleotides of this type are available today through established vendors, such as Twist Bioscience Corporation, of San Francisco, Calif., U.S.A.

In preferred implementations, at each “gene-univ” junction of each single-stranded concatenated polynucleotide, the “gene” and “univ” segments may be selected so as to create or fashion a restriction enzyme binding/cleaving site (see a plurality of sites 505, indicated as arrows in FIG. 5A). For example, for a restriction enzyme that cleaves in the middle of its binding site creating “blunt” ends (e.g., EcoRV, which binds to GATATC), the last three bases of the locus primer binding site chosen for gene1 may contain the first three bases of that six-base pair binding site (i.e., GAT), and the first three bases of the universal primer binding sequence may be chosen to contain the last three bases of the restriction enzyme binding site (i.e., ATC). As another example, for a restriction enzyme that cleaves between the fourth and fifth base of its binding site leaving “sticky” ends (e.g., as Kpn1), the last five and first five bases of the “univ” and “gene” sequence segments, respectively, may be created or fashioned to conform to the restriction binding site GGTACC, such that the 3′ end of the gene binding site is GGTAC, and the first base of the universal primer sequence is C.

Notably, each single-stranded concatenated polynucleotide is provided with a moiety on its 5′ end to facilitate a subsequent binding of the polynucleotide concatenate to a (e.g., cell) binding agent. In some implementations, the moiety is biotin. In the illustrative example, the binding agent is an antibody which may recognize an epitope found on the cellular/nuclear membrane of some of the cell types in the sample, and has at least one conjugated binding site for the 5′ end moiety (e.g., biotin) of the polynucleotide concatenate. As biotin binds to streptavidin, the antibody may be conjugated to streptavidin.

As shown in FIG. 5A, a microtiter plate 510 having a plurality of wells may be utilized in the process as will be described. In the illustrative example, microtiter plate 510 has a 96-well plate format with 96 wells. In other implementations, a microtiter plate having a 384-well plate format with 384 wells (or higher) may be utilized, or alternatively even non-plate formats (e.g., nanowells) or other devices that separate samples from one another may be utilized.

In the illustrative example, the binding agent that is utilized in the process may be an antibody. In other implementations, other agents or chaperone entities may be utilized. For example, specific proteoglycans or glycoproteins may be utilized depending on the application, or even lipid vesicles for indiscriminate microdroplet targeting irrespective of the cell or cell type inside.

Even further in the illustrative example, an antibody epitope that is differentially-expressed amongst cells of the samples, such that only a fraction of the cells of each sample are targeted, may be utilized. In other implementations, a ubiquitously-expressed antibody present on the membranes of all cells or nuclei may be utilized. With respect to the illustrative example of the antibody epitope that is differentially-expressed, certain alpha or beta integrin chains, such as beta 1, alpha 2 and alpha 3, which are regionally-expressed among epithelial cells depending on their position in various epithelia, including oral epithelium and with beta 4 subunits, in gastric epithelium, may be used to select only a fraction of the epithelial cells in each sample. With respect to oral epithelium, see, e.g., Thorup A, Dabelsteen E, Schou S, Gil S, Carter W and J Reibel; “Differential expression of integrins and laminin-5 in normal oral epithelia,” APMIS, 1997, July; 105(7):519-30. With respect to gastric epithelium, see, e.g., Virtanen I, Tani T, Back N, Happloa O, Laitinen L, Kiviluoto T, Salo J, Burgeson R, Lehto V and E Kivilaakso; “Differential expression of laminin chains and their integrin receptors in human gastric mucosa,” Am J Pathol. 1995; October; 147(4):1123-32.

Indeed, many of the integrin subunits involved in basal membrane adhesion may be regionally-expressed within anatomically-defined epithelia. Alternatively, one could target CD138+ (e.g., mature, circulating) B-cells from whole blood evidence per samples (CD138 is expressed on the cell membrane of such cells), or CD1 positive cells indicative of epithelium, or CD54 positive cells indicative of endothelium, or CD340/HER-2, a well-known epithelial tumor antigen expressed on the surface of many breast and ovarian tumor cells. With respect to CD1 positive cells indicative of epithelium, see, e.g., for a comprehensive list of such useful antigens, at https://www.sinobiological.com of Sino Biological US Inc. of Chesterbrook, Pa., U.S.A., or more specifically, https://www.sinobiological.com/research/d-antigens/epithelial-cell. With respect to CD340/HER-2, see, e.g., Mitri and O'Reagan, “The HER2 Receptor in Breast Cancer. Pathophysiology, Clinical Use, and New Advances in Therapy,” Chemotherapy Research and Practice,” 2012, 2012:743193. Doi:10.1155/2012/743193.

Continuing in the flowchart 400 of FIG. 4, each one of the plurality of single-stranded concatenated polynucleotides (e.g., synthesized as described above) is placed in a corresponding (unique) well in the microtiter plate (step 408 of FIG. 4). In FIG. 5A, where microtiter plate 510 having 96 wells is utilized, each one of 96 such polynucleotide concatenates may be placed in a corresponding (unique) one of the 96 wells. For example, single-stranded concatenated polynucleotide 504a (associated with “bc1”) may be placed in a well 512, single-stranded concatenated polynucleotide 506a (associated with “bc2”) may be placed in a well 514, and single-stranded concatenated polynucleotide 508a (associated with “bc3”) may be placed in a well 516. In the illustrated example, each one of the single-stranded concatenated polynucleotides associated with its unique barcode has 96 gene sequences (“gene”) corresponding to the 5′ ends of the 23 target CODIS loci. Thus, each well may now contain a polynucleotide concatenate associated with a unique barcode region but, other than the barcode regions, the polynucleotide concatenates amongst the wells may be identical.

Next, the plurality of single-stranded concatenated polynucleotides in each well of the microtiter plate may be converted into double-stranded form (step 410 of FIG. 4). The conversion of the polynucleotide concatenates into double-stranded form may be performed with use of the reverse complement of the 3′ most terminal element's gene sequence for binding the extension primer. To each well of microtiter plate 510 of FIG. 5A is added the primer, DNA polymerase, and dNTPs, so that extension may take place.

In FIG. 5C, what is shown is an example illustrative representation of the conversion of the single-stranded concatenated polynucleotides into double-stranded DNA. More particularly, FIG. 5C shows a plurality of double-stranded concatenated polynucleotides 502b which include a double-stranded concatenated polynucleotide 504b, a double-stranded concatenated polynucleotide 506b, and a double-stranded concatenated polynucleotide 508b. The box-like structures (e.g., box-like structures 520 and 522 of concatenated polynucleotide 504b) are used to indicate its resulting double-stranded nature.

In some implementations, this terminal sequence utilized in this step may be the last gene in the set, in which case the sequence that is complimentary to this sequence may be used as the primer. In alternative implementations, the terminal sequence may be a second universal sequence at the 5′ and 3′ ends of the polynucleotide concatenate. See, e.g., concatenated polynucleotides 502a of FIG. 5A, as well as concatenated polynucleotide 504a of FIG. 5B. In other alternative implementations, the terminal sequence may be a universal sequence at only the 3′ end of the polynucleotide concatenate, provided specifically for this purpose, in which case the reverse complement of the universal sequence may constitute the extension primer. See, e.g., the concatenated polynucleotide having the additional universal primer binding site (e.g., “univ_3”) at its ‘3 end at the bottom of FIG. 5B.

Next, a binding agent (e.g., a chaperone molecule or entity) may be added to each well of the microtiter plate (step 412 of FIG. 4). In some implementations, the binding agent may be an antibody (Ab). In FIG. 5D, what is shown is an example illustrative representation of the addition of the binding agent (e.g., a binding agent 530 associated with double-stranded concatenated polynucleotide 504b) to each well of microtiter plate 510. As mentioned previously, the antibody will later recognize an epitope found on the cellular membrane (or nuclear membrane if using nuclei instead of cell samples) of some of the cell types in the sample, and may have at least one conjugated binding site for the 5′ end moiety of the polynucleotide concatenate.

In the illustrative example, the 5′ end moiety is biotin, which binds to streptavidin, and therefore this antibody may be conjugated to streptavidin. Such streptavidin-conjugated antibodies are well-known to those familiar with the state-of-the-art and are readily available from a variety of vendors; even kits for preparing streptavidin-conjugated antibodies are available. See, e.g., https://www.bio-rad-antibodies.com of Bio-Rad Laboratories, Inc. of Hercules, Calif., U.S.A., or more particularly, https://www.bio-rad-antibodies.com/kit/streptavidin-conjugation-kit-lnk16.html?f=kit. Streptavidin may be conjugated randomly to lysine residues of the antibody with an average of two molecules of streptavidin per antibody. See, e.g., Hoffmann et al., “Rapid conjugation of antibodies to toxins to select candidates for the development of anticancer Antibody Drug Conjugates (ADCs),” 2020, Sci Rep 10, 8869.

Next, different samples which comprise cells or nuclei (depending on the application and antibody epitope or agent binding specificity) may be placed into the microtiter plate (step 414 of FIG. 4). More particularly, each sample may be placed into its own well of the microtiter plate, where the binding agents bind to their cellular/nuclear epitopes of particular cells/nuclei of the sample. In FIG. 5E, what is shown is an example illustrative representation of the addition of a cell 540 of a sample 541 (which includes a plurality of other cells as shown around a bracket) to a well of microtiter plate 510, where binding agent 530 of concatenated polynucleotide 504b may bind to the epitope of cell 540. As indicated, the sample 541 of the cells which include cell 540 are uniquely identified by barcode “bc1.” Also shown in FIG. 5E is the addition of a cell 542 of a sample 543 (which includes a plurality of other cells as shown around a bracket) to a different well of microtiter plate 510, where a binding agent 532 of concatenated polynucleotide 506b may bind to the epitope of cell 542. As indicated, the sample 543 of the cells which include cell 542 is uniquely identified by barcode “bc2.” After this binding process, reaction clean-up is optional.

Thus, different concatenated polynucleotides from different samples (e.g., concatenated polynucleotide 504b of sample 541 and concatenated polynucleotide 506b of sample 543 of FIG. 5E) may be distributed to different wells of the microtiter plate, where each is bound to an antibody, and where each antibody, through an epitope commonly found on the surface of cells or nuclei, is bound to a cell or nucleus such that in each well, each or most or many of the cells are bound by an antibody with a common polynucleotide and integrated barcode identifier. Each well contains a different sample of cells and a polynucleotide concatenate with a unique barcode region bound to the antibody. All of the cells of the sample may be bound if an antibody recognizing a ubiquitously-expressed epitope is used, or only some of the cells of each sample may be bound to the antibody if it is engineered to recognize a differentially-expressed epitope such as CD34+ white blood cells, or a tumor antigen, or if proteoglycan or glycoprotein agent is used, a specific binding profile.

Next, the contents of each well of the microtiter plate may be pooled into a single tube or well (step 416 of FIG. 4). Though all of the cells of the different samples are mixed, each cell is bound to an antibody with a linked polynucleotide containing a “sample-specific” identifying barcode integrated into its sequence. In FIG. 5F, what is shown is an example illustrative representation of the pooling of the contents of each well (e.g., cell 540 which is bound to concatenated polynucleotide 504b and cell 542 which is bound to concatenated polynucleotide 506b and others from different samples) into a tube 546. Again, clean-up is optional at this point.

Thus, the cells from all of the different samples may be pooled. In at least some cases, each cell of each sample will be bound to an antibody and, in the illustrative example, only some of the cells are bound because the epitope is differentially-expressed amongst cells within each sample. The antibody may be linked to a double-stranded polynucleotide concatenate containing an integrated barcode that is indicative of the well from which the cells or sample came (e.g., bc1, bc2, bc3, etc.).

Next, the antibody-bound cells/nuclei are then injected, fused, or otherwise integrated into microvesicles (e.g., microreactors, microdroplets, emulsion, etc.) together with the bead library (step 418 of FIG. 4). This process may be performed, for example, with use of state-of-the-art microfluidics methods provided through various vendors. Here, the objective is to achieve one antibody-bound cell/nucleus encapsulated with one bead in each microreactor, which these machines are known to reliably achieve. In FIG. 5G, what is shown is an example illustrative representation of a microdroplet 550 having cell 540 which is bound to concatenated polynucleotide 504b with binding agent 530, together with a bead 550a and its oligos 552a. In some implementations, univ2 primers may be injected at this time, and a pre-amplification step using the univ2 primer sequences may be used to better balance the bead-provided primers with those provided bound by the antibody in subsequent steps. In FIG. 5G, univ and univ2 primers are also shown with concatenated polynucleotide 504b prior to linear amplification.

Thus, what is provided is an encapsulated, cell-bound antibody linked to a double-stranded polynucleotide concatenate containing 5′ primers (e.g., or 3′ primers), where the polynucleotide concatenate has integrated a barcode indicative of its sample origin, along with a vendor-supplied bead that is linked to oligonucleotides containing the ‘3 primers (e.g., or 5′ primers) for the targeted loci. Within the bead primers is integrated a different barcode indicative of the cell, bead, and emulsion (indicated with green “G” color in FIG. 5G, optionally with a UMI present in the barcode). If the antibody is linked to the 5′ primers, the bead is linked to the 3′ primers for the target gene panel; and if the antibody is linked to the 3′ primers, the bead is linked to the 5′ primers for any given member of the target gene panel. Not all of the cells are bound by the antibody (as per FIG. 5E), but those that are not bound and are incorporated with a bead minus the polynucleotide and, as such, will not produce amplification products. In this step, restriction enzyme and an excess of univ primer sequences may also be added to each microdroplet.

The following steps may be performed in any one of a number of different alternative orders. When components such as enzymes, buffers, or reagents (e.g., dNTPs) are added to microdroplets, there are any one of a variety of different means by which this processing may be accomplished. For example, means described in various background/related art may be utilized, for example, processing associated with U.S. Pat. No. 10,501,739, which may include the addition of the components in the fluid that is initially encapsulated with a cell and/or bead, the merging of emulsion microdroplets through the use of microfluidic junction nodes, the contacting of a microdroplet with a solution containing the component wherein the component enters the microdroplet through diffusion (perhaps even active transport), the injecting of the microdroplet with a solution containing the component, and/or the flowing of the component into a carrier fluid comprised of microdroplets.

The double-stranded concatenated polynucleotide may be digested with restriction endonuclease which is added to each microdroplet (step 420 of FIG. 4). At the same time, an excess of univ oligonucleotides, DNA polymerase, and dNTPs may be added. Here, digestion by the restriction endonuclease results in the release of individual, “univ-bc-gene” elements of the polynucleotide concatenate, which was previously a concatenate of these elements. In FIG. 5H, what is shown is an example illustrative representation of microdroplet 550 after the addition of the restriction endonuclease and other items, showing the release of individual, “univ-bc-gene” units 560a from the polynucleotide concatenate, as well as the univ and univ2 primers.

The excess univ primers may then be used in linear amplification for creating an excess of “sense” univ-bc-gene strands for each element (step 422 of FIG. 4). This process step may involve cycling between annealing and extension temperatures, which is desirable because the elements are double-stranded, and the bottom or “antisense” strand (i.e., relative to our PCR orientation, where “sense” extends toward the target locus and “antisense” would extend away from it) competes with the genomic DNA for the sense strand during the annealing steps. In FIG. SI, what is shown is an example illustrative representation of microdroplet 550 after the creation of an excess of “sense” univ-bc-gene strands 560b for each element, which are the polynucleotide primers to be subsequently utilized. In the figure, the single-stranded nature of the elements is indicated by drawing them as lines instead of boxes.

Next, proteinase may be added to the microdroplets for disrupting of the nucleosome/DNA structures, thereby allowing access to the genome (step 424 of FIG. 4). As described previously in the state-of-the-art, it is beneficial to add proteinase in targeted DNA applications for these reasons. Here, the nuclear membrane may be disrupted, either osmotically and/or with assistance of the proteinase, and the nucleosomal proteins may be digested, releasing the genomic DNA. In FIG. 5J, what is shown is an example illustrative representation of a proteinase 565 which is added to microdroplet 550, which disrupts the nucleosome/DNA structures for releasing the genomic DNA.

In some implementations, the polynucleotide concatenate may include a dummy element at its 5′ end (see, e.g., FIG. 5B that shows polynucleotide concatenate 504a), and after the preceding restriction digestion of the polynucleotide concatenate, each univ-bc-gene element may be released from the antibody. In other implementations, where the polynucleotide concatenate does not include the dummy element, but rather uses another form (see, e.g., FIG. 5B that shows the top-most polynucleotide concatenate), the proteinase may release the first element which remained bound to the antibody. In any case, the proteinase may be inactivated through denaturation after this step, so that it does not degrade proteins utilized in subsequent steps.

The bead may then be degraded or dissolved, for releasing the oligos or primers (step 426 of FIG. 4). In some implementations, digestion of the bead may be performed with agarase if composed of agarose, or with ultraviolet (UV) irradiation if the bead has UV-sensitive material. In FIG. 5K, what is shown is an example illustrative representation of a dissolved or degraded bead 550b with its released bead library oligos or bead primers 552b.

A thermostable DNA polymerase and dNTPs may then be added, and targeted PCR is carried out for amplifying the target gene panel (step 428 of FIG. 4). In some implementations, genomic amplification may be performed according to standard practice. In FIG. 5L, what is shown is an example illustrative representation of amplification products 570 which are the result of the primers being used with PCR on the released DNA or cDNA. Bead primers 552b constitute the 5′ set of primers for the target gene panel, whereas univ-bc-gene strands 560b are the polynucleotide primers which constitute the 3′ set of primers for the target gene panel. Amplification products 570 contain barcodes indicative of the cell of origin at their 5′ end, and indicative of the sample of origin at their 3′ end. Amplification products 570 are the result of three different targets derived from one cell (indicated with green “G” barcode section in 3′ end of the amplicons) of an identified sample (indicated with “bc1” in 5′ end of amplicons).

Thus, what are produced are amplification products where the polynucleotide barcode integrated into the primer at the 3′ end (or 5′ end) informs as to the sample (e.g., microtiter plate well) and the bead library barcode integrated into the primer present at the 5′ end (or 3′ end) informs as to the cell within that sample. Notably, the amplification products of the sample multiplex are obtained from only a subset of cells in each sample.

In FIG. 5M, what is shown is an example illustrative representation of sets of amplification products 570, 572, and 574. Here, cell-specific barcodes are indicated with different symbols or shapes in the second element of the 5′ portion of each amplicon, whereas sample-specific barcodes are indicated with different symbols or shapes in the 3′ region of each amplicon. Here, each set of three gene amplicons from the same sample is derived from a microtiter well, having a common 3′ barcode. No two cells in the pool should have the same bead/cell barcode-sample barcode combination (e.g., only one star-magenta amplification product in the entire set). Of course, many cells within each sample/well may contain the same polynucleotide concatenate-derived barcode.

As is apparent, all of the amplification products may be pooled into a single tube and, not only is the information built into the amplification products informative as to the cell of origin, but the sample of origin as well. This has been accomplished as a “skim” on each sample, without wasting enzyme/reagents or generating amplicons for every cell of the sample. Because only a portion of the cells of each sample were sampled, what is produced is a “shallow” set of amplification products for each sample, enabling more samples to be combined or stacked into a well or channel of vendor-provided equipment and cartridges.

In this way, multiple samples (e.g., 96, 384, or more, depending on the plate configuration) may be combined for single runs through vendor-provided emulsion generation and NGS systems to reduce costs associated with these runs. This is the major driver in per-sample SCNGS costs, especially the former. Each of these runs requires consumables and reagents that are the primary determinants of per sample costs, and, dividing these costs by a large number of samples enables a lower per sample cost.

The barcodes now serve two purposes, not just one. In particular, they resolve the cells within the sample in addition to the well or sample identity. Therefore, all samples may be processed together in a few hours, consuming a single cartridge and reagent kit, and submitted to NGS at the same time to thereby consume only one channel of the NGS kits, where the resulting sequences may be computational-attributed (in a foolproof or guaranteed manner) to both cells and samples of origin during analysis.

With reference back to FIG. 3, the value in at least some implementations of the present invention may now be better appreciated. In some implementations, as described earlier above, the present invention may constitute a series of steps in the form of module 350 that fits into and is inserted into the workflow 300 as indicated in FIG. 3 (see the arrow which inserts the module 350 into workflow 300). The series of steps of module 350 may be performed prior to loading samples into the cartridge channels, involving enhanced sample tagging which may be inserted prior to library preparation process 306 of FIG. 3. The enhanced process of module 350 may be performed using the superior molecular biology approach that allows for “skimming” of the sample of cells so that channel bandwidth is preserved for analysis of far larger numbers of samples simultaneously. In some implementations, with use of module 350 in the workflow 300, the workflow process may be adjusted to produce data at about $2-$8 per sample with a throughput that is greater than 3,000 samples per week.

A preferred embodiment of the type of product that may be provided is a 96, 384 or higher-well plate, within which are dried-down, antibody-polynucleotide concatenates, ready for cellular sample addition. In some implementations, these may be made to order, for the target genes of interest, or alternatively may be standardized for specific applications. In forensic science applications, as the evidence presented almost always contains one or more of epithelial, blood, and semen cells, it may be advisable to include antibodies recognizing epitopes for certain epithelium, white blood cells and semen, to “skim” each sample in a manner inclusive of the possible cell types that may be present and avoid type II (false negative) error.

Rather than load sample cells into the cartridge channel directly, the user may load the sample cells into the microtiter plate wells containing the antibody (or other agent) bound polynucleotide concatenates, incubate, pool together, and then load into the cartridge channel.

Prior to the present inventive work, the problem that limits SCNGS market growth with the current state-of-the-art was:

1) Throughput—only eight (8) samples per week per machine; and

2) Cost—from $687 (low-order multiplexed 8-fold)-$5,000 per sample (not multiplexed).

For example, drug discovery research requires large numbers of pre- and post-drug SCNGS datasets (one for pre-treatment cells, one for post-treatment cells). A screen of 384 drug compounds would require 768 tests, take 96 weeks with one machine, and cost $2M. This kind of experiment is simply not feasible for routine drug discovery screening requiring hundreds of such plates. As another example, in forensic science, the current gold-standard, which suffers from sensitivity and resolution problems, is $40 per sample, with a throughput of about four-hundred (400) samples per week per machine. Even though SCNGS is the solution to the “mixture problem” and “sensitivity problem”, the community simply has not adopted at $5,000 per sample and eight (8) samples per week per machine throughput because these costs and throughput are simply not feasible for routine casework requiring several hundred samples be analyzed affordably each week.

On the other hand, in some implementations of the present disclosure, what may now be achieved is:

1) Throughput—three-hundred eighty-four (384) samples or more per week.

2) Cost—$2-$30 per sample.

In the first drug discovery described above, a screen of 384 drug compounds would require 768 tests at a cost $7,500 and take about one week to complete. Such costs and throughput are feasible for routine screening that requires hundreds of such plates. In the forensic science example, the costs at $3-$30 per sample and 384 samples per week per machine throughput are better than current gold standard of $40 per sample and 384 samples per week per machine, while solving the sample mixture and sensitivity problems plaguing the industry, making it feasible for routine casework requiring several hundreds of samples analyzed economically each week.

Note that, for vertical applications of SCNGS, it may not be advisable to multiplex samples too extensively, lest the channel depth of the NGS system be exceeded and the desired read depths per cell not achieved. The commercially-available limit presently available with background/related art cell multiplexing systems, which tag all cells of a sample indiscriminately, is eight (8). These methods utilize lipid vesicles containing oligonucleotides with N “bc”-“univ” sequences, where N is the number of samples, and a bulk set of 3′ primers (or 5′ primers) containing the “univ” sequence at its 5′ end. The lipids fuse with cells which are then partitioned into microdroplets with bead bound 5′ primers (or 3′ primers) and the resulting amplification products incorporate the “bc”-“univ” tags, thereby identifying the sample. The problem with this approach is that, without cell counting, the samples with higher cell densities will “hog” channel bandwidth relative to those with lower cell densities. And, due to the stochastic nature of polymerase chain reaction, cells from the sparser samples may be out-competed for polymerase and primers, resulting in their signal being lost in the NGS data. This may be the reason why the method is limited to multiplex factors of eight (8).

In elevating this limit substantially, the present inventive work has opened up horizontal SCNGS as a more efficient and cost-effective version of horizontal SCNGS, where channel depth is not as important as costs per sample. With horizontal SCNGS, one is interested in finding exemplars of cellular diversity for diagnostic or forensic identification purposes, not in sequencing each and every cell of a particular subtype or origin. Thus, what is traded off is the depth of information from each sample in exchange for the ability to process an increased number of samples simultaneously, as it enables the achievement of lower per sample costs.

Existing applications of horizontal SCNGS for targeted DNA sequencing waste much of the channel depth on redundancy, as they are only pseudo-horizontal in that, while they target a limited number of loci and allow for sample multiplexing, the amplification products are produced from all of the cells of the sample indiscriminately and the degree of multiplexing achievable is low. This follows from its design, which was originally tailored to vertical SCNGS applications that value (and must preserve) sample depth (e.g., the number of reads per cell and number of cells per sample). In the background/related art, the multiplexed, horizontal SCNGS uses bulk primers as companions to those found on the beads, and thus produce data for all cells in the sample indiscriminately, a la vertical SCNGS. With forensic and diagnostic applications designed to skim a sample for the presence of a major component (such as a donor, or a cell type), these methods are still too expensive and laborious. If the methods were more efficient at using the channel depth, they could lower the costs to implement them on a sample-to-sample basis. Lower cost points would enable widespread adoption for routine diagnostics and human identity tasks, which are characterized by high volume requirements and low-cost demands. Advantageous, the present inventive work provides the first such system to enable cost-effective horizontal SCNGS for these types of higher-throughput applications.

In some implementations, steps 404 through 428 of FIG. 4 may be carried out in a slightly different order to accomplish the same end objective in the last step. For example, depending on the details (e.g., whether the proteinase activity is inhibited and activated), restriction endonuclease digestion may be carried out before or after bead digestion or proteinase digestion, or the bead digestion step can occur before or after the proteinase digestion without substantive difference.

In some implementations, it may be useful to stoichiometrically balance the 5′ and 3′ primer sets on the beads and antibodies or other agents, especially if each antibody/agent is bound to only one polynucleotide concatemer providing one (e.g., the 3′ primer) set, since the number of 5′ primers contributed by the beads will be contributed in far greater proportions per gene sequence. To accomplish this, the univ2 primers of FIG. 5A (middle) may be used to pre-amplify the polynucleotide concatenate inside of the microdroplets prior to restriction endonuclease digestion. The univ2 sequences may be the same, or they may be different, as long as the appropriate sense and anti-sense primers are used to accomplish pre-amplification.

In some implementations, the method can be applied with equal efficacy, with or without the univ2 adaptors, if instead of a single polynucleotide concatemer being bound to the antibody, multiple polynucleotide concatenates are bound to multiple binding sites on the antibody. If the number of binding sites equals the number of target loci and gene sequences, the number of elements in the polynucleotide concatemer N may be one, as long as a set of elements covering all of the gene sequences required is used. In this case, there would be no need to digest the antibody bound nucleotide with the restriction enzyme and instead, one may rely on the protease to release the units for amplification through digestion of the protein antibody.

In some implementations, the method may be applied to whole transcriptomic SCNGS. Here, instead of using a concatemer of “univ-bc-gene” elements, a single “univ-bc-rand” element may be utilized, where “rand” refers to a randomized N-mer oligonucleotide (where, e.g., N may be 6, 8, 12, 20, etc.). If a sufficient number of random sequences are present in the randomer, the nucleotide, once liberated from the antibody, will prime for most of the cDNA sequences generated by vendor-supplied bead libraries using poly dT as the “gene” sequence.

In some implementations, the antibodies used may be monoclonal or polyclonal. Polyclonals may provide assurance that human variation in epitope expression does not result in type II (false negative) results for a given donor to the mixed forensic, R&D, or clinical diagnostics sample. Monoclonals that demonstrate low type II error may be desirable for quality assurance purposes, since antibody-epitope binding can be more easily qualified and quantified.

In some implementations, the process may include the introduction of enzymes and reagents into microdroplets. The likelihood of two microdroplets or a micro and nanodroplet fusing is a function of, in part, their size and concentration. What is desirable is to minimize cell and bead containing microdroplets from fusing, but facilitate the smaller restriction enzyme containing nanodroplets fusing with the cell and bead containing microdroplets; one could utilize a concentration of the former in excess of the latter.

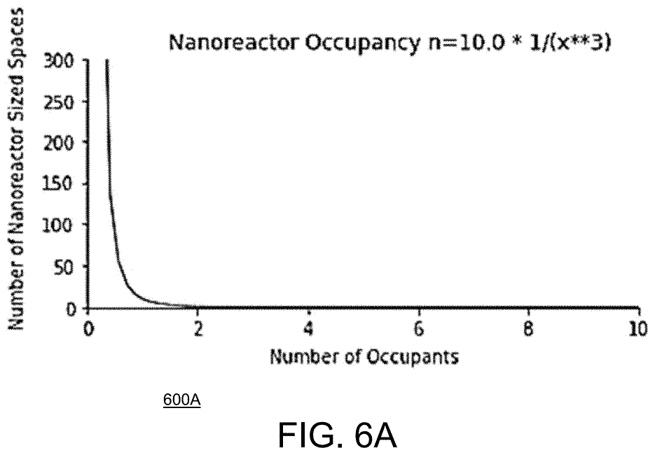

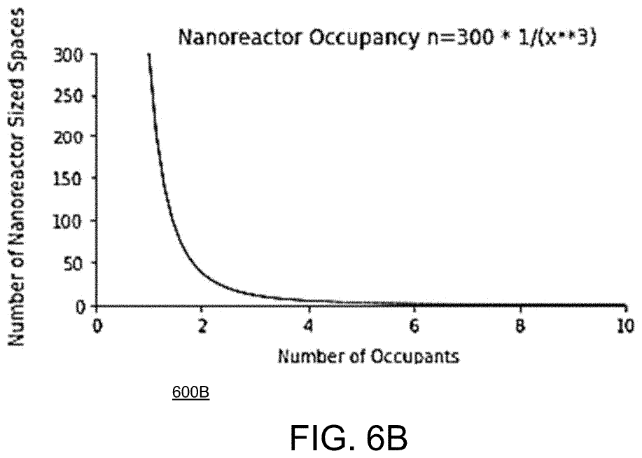

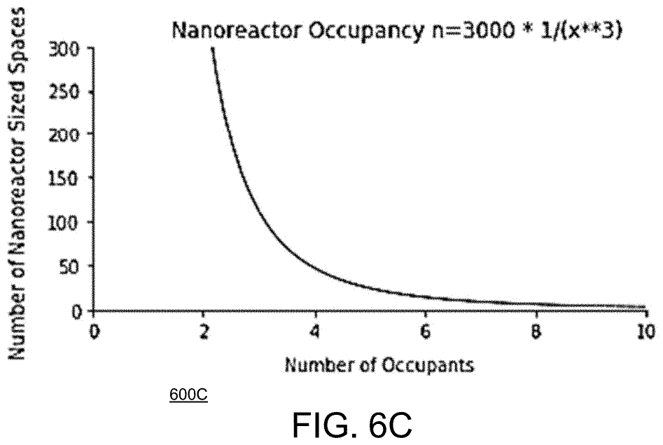

As part of the development of the present inventive work, a software algorithm for modeling statistical dilution of components of unequal size has been developed to help manage the fusion goals. FIGS. 6A, 6B, and 6C are plots 600A, 600B, and 600C of a function n*(1/x³) which show similar forms taken for various values of n as that obtained with the program written for the optimization of statistical dilution steps. Each one of plots 600A, 600B, and 600C depicts the relationship between the number of occupants and the number of nanoreactor-sized spaces for different values of n (e.g., where n is 10, 300, and 3000, respectively). What changes from plot to plot is the total number of microdroplet (microreactor or nanoreactor) sized spaces in solutions that are occupied with a microreactor and a lipid nanodroplet containing enzyme and reagent (“n”). The distribution of the data takes on the general form of n*1/x³.

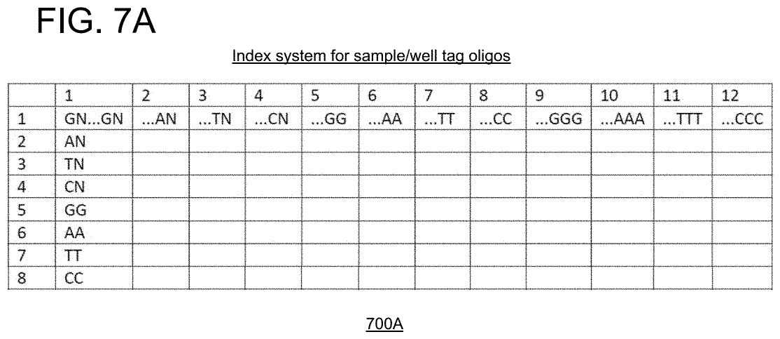

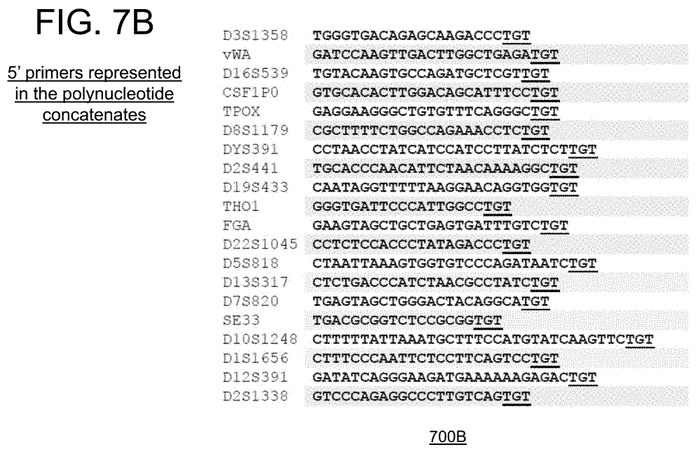

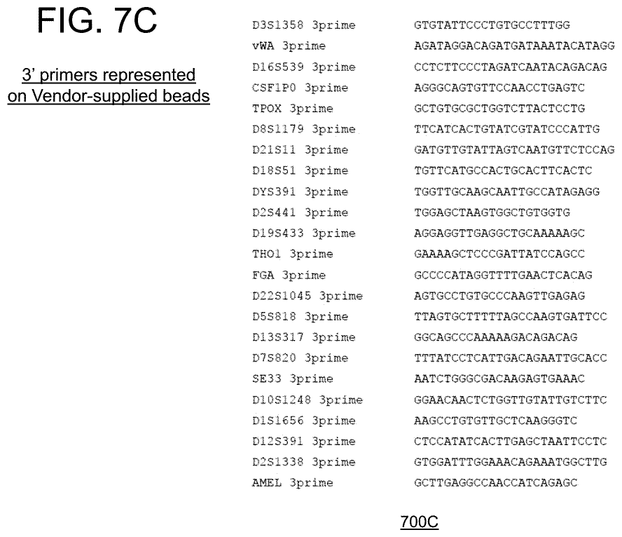

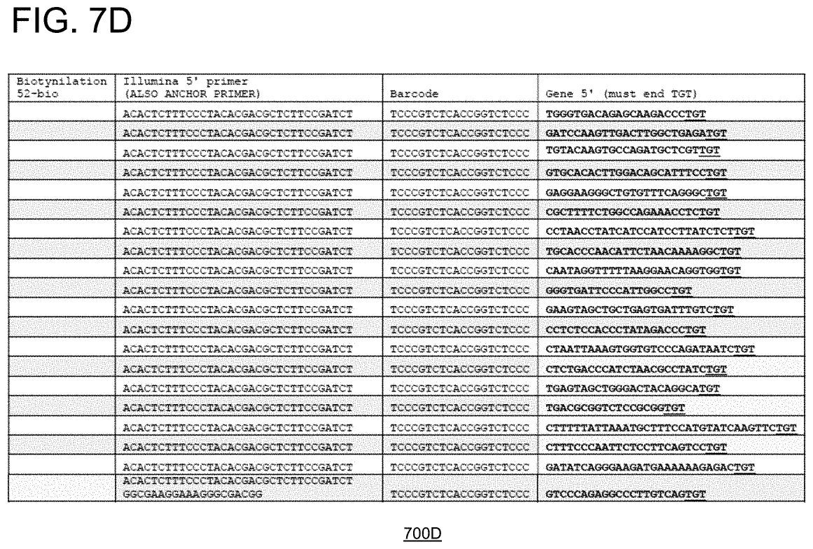

An index system incorporating a cipher may be utilized for selecting the random barcodes of the polynucleotide concatenates used to tag the sample identities. Such a selection may be performed so that bioinformatically-attributing sequences to the appropriate sample may be foolproof or guaranteed. For example, consider a grid 700A shown in FIG. 7A, which is indicated as an “Index system for sample/well tag oligios.” Here, the presence of a G and a nucleotide that is not G (N) in positions 1 and 2 of the barcode is indicative that the sequence belongs to a sample in row 1 of a 96-well plate. The presence of a GN, AN, TN or CN in positions 3 and 4 indicate the sequence belongs to a sample in columns 1, 2, 3 or 4, respectively. Thus, the barcode sequence GTAT would correspond to row 1, column 2, and the barcode sequence GGCC would correspond to row 5, column 8. The value of using such an index system when selecting random barcode sequences is that it standardizes the designation of sample identity in nucleotide form, such that downstream data quality may always be confirmed (even visually) and mistakes in downstream analyses, such as those which may arise due to software bugs, can be easily recognized (even visually).

In order to further demonstrate the creation of the polynucleotide concatenates, a software program has been written. In forensic science, the polynucleotide concatenates may incorporate Tm matched primer sequences for the CODIS set of STR loci, such as those shown in a table 700B of FIG. 7B (i.e., the 5′ primers) and a table 700C of FIG. 7C (i.e., the 3′ primers). In this example, the 5′ primers are incorporated into the polynucleotide concatenates (i.e., per FIG. 7B), and the 3′ primers are incorporated on the vendor-supplied beads (i.e., per FIG. 7C). The software program was designed to identify Tm matched primers at each of the CODIS STR loci, combine them with a commonly used “univ” sequence, twenty (20) random barcodes (e.g., see discussion above in relation to FIG. 7A), and assemble them into polynucleotide concatenates, where the univ-gene junctions contain a BsrG1 restriction digestion site (TGTACA).

Before concatenation in the polynucleotide, each primer may be joined with a universal and barcode sequence as shown in a table 700D of FIG. 7D. One will note that the terminal 3 nucleotides of each gene sequence is TGT, and the first three (3) nucleotides of each univ sequence is ACA, such that the union of each gene sequence with each universal sequence forms the TGTACA restriction site. One will also note that the barcode sequence is the same for each element. Accordingly, these elements are combined to produce a list of twenty (20) polynucleotide concatenate sequences, shown in the List of Polynucleotides at the end of the specification (“Polynucleotide 0” through “Polynucleotide 19”; one concatenate for each of the twenty barcodes). Polynucleotide 0 of this list is that formed by the precise elements of table 700D of FIG. 7D; the remaining nineteen (19) polynucleotides are the same except they incorporate a different barcode sequence. The type of polynucleotide concatenate used here is the (univ-be-gene)ₙ-univ2 type, where n=23, which is the number of CODIS loci. The example synthesis as revealed in the List of Polynucleotides at the end of the specification may allow for a microtiter well plate of twenty wells to be assembled with antibodies for multiplexing of twenty different forensic samples.

Continuing on with the disclosure, note that, in forensic science applications, so-called “touch DNA” samples may contain very low numbers of epithelial cells derived from human skin. Here, the cell numbers may be so low that it may be possible to use an antibody that recognizes a ubiquitously-expressed epithelial, or general mammalian cell membrane epitope. This may be realized without crowding the reagent/channel space too much to prevent the scale of “stacking” or multiplexing achievable with differentially-expressed epitopes, which allow for “skimming” of a sample.

In at least some cases, it may not be possible to isolate intact cells from forensic evidence, since it is usually presented in a dried format (e.g., a dried swab), and therefore, the inventive processes may be only useful in forensic science with nuclei, in which case the antibody epitope would need to be a nuclear epitope. The use of a nuclear epitope would likely make it much less likely or even impossible to use a differentially-expressed epitope for “skimming” of the sample, but the sparse nature of cells present on dried evidence swabs are almost always present in vanishingly small quantities. This would allow substantial stacking or multiplexing of samples without any one sample crowding too much of the “channel space” with its amplification products. The sparse cells present on dried evidence swabs may include, for example, epithelial cells from “touch DNA” evidence, white blood cells from whole blood evidence, and semen cells from Sexual Assault Evidence Kits (SAEKs).

The same applies to whole blood evidence, since only white blood cells contain nuclei, and white blood cells are present at low levels compared to red blood cells and platelets. Using a circulating B-cell epitope, such as CD138, will always allow for greater multiplexing capability and lower costs, however, without sacrificing quality since the nature of SCNGS in providing millions of reads per experiment, one still expects a large enough number of exemplars from each donor to be confident in the profile generated.

With fresh buccal or saliva samples, notorious for high numbers of cells per sample, epitopes corresponding to ubiquitously-expressed epithelial gene products would be advisable, such as cell adhesion molecule (CAM) epitopes, tight junction, desmosome, gap junction, or because all epithelial cells bind to the extracellular matrix through fibronectin via integrin proteins, integrin epitopes. Dried buccal samples would probably need nuclei dilution prior to adding to a set of samples, lest it potentially crowd out other samples, or consume too much of the channel space with its amplification products.

Thus, as described above, the inventive process may involve a way in which to “skim” or take a small fraction of cells of a given sample so that samples can be “stacked” or multiplexed prior to injection into microreactors, such that their amplification products contain barcodes enabling attribution to specific cells as well as the sample of origin. However, one might consider why the use of the agent-bound polynucleotide concatenate method is preferable to merely taking a small fraction of each sample, stacking or multiplexing these small fractions, and using the background/related art of lipid nanoreactor or antibody bound to a small tag oligonucleotide. Here, the small tag oligonucleotide contains a universal sequence and becomes incorporated into the amplification products generated by bead coated primers and ubiquitously provided primer partners.

In response to this inquiry, the latter, background/related art method may indeed be easier and involve fewer steps, but it does not allow for high-order stacking or multiplexing unless even numbers of cells from each sample are taken, which requires cell counting which is a laborious process. Without cell counting, the samples with higher cell densities will “hog” channel bandwidth relative to those with lower cell densities. And, due to the stochastic nature of polymerase chain reaction, cells from the sparser samples may be out-competed for polymerase and primers, resulting in their signal being lost in the NGS data. Indeed, this is likely the reason why the current vendors that practice this method of multiplexing only allow for eight-sample combinations in each channel.

In at least some implementations of the present disclosure, such problems of the background/related art may be avoided by using an agent-bound polynucleotide concatenate (e.g., referred to as “antibody-bound polynucleotide concatenate” for simplicity) as a gatekeeper, wherein only cells that bind an antibody-bound polynucleotide concatenate will produce amplification product. Since the moles or numbers of antibodies or antibody-bound polynucleotide concatenates in each well can be easily equilibrated, the use of antibody-bound polynucleotide concatenates effectively normalizes each samples contribution to the downstream amplification products, such that each sample consumes the same fraction of the channel bandwidth.

A good example of the benefit of this effective normalization is with SAEK commonly encountered in forensic science. With SAEKs, there is a need to separate sperm cells from epithelial cells. Swabs are taken from body orifices, and often contain far more victim epithelial cells than suspect sperm cells. For this reason, a differential lysis procedure is most commonly used to separate the two cell types prior to analysis with capillary electrophoresis. In at least some implementations of the present disclosure, by using antibodies with epitopes expressed on the surface of the sperm cell, this laborious process could be eliminated, since the epithelial cells in the sample mixture would not produce amplification products contributing to the SCNGS generated CODIS profiles. Further, since some items of evidence may contain a greater number of sperm cells than others, the use of the same number of antibody-bound polynucleotide concatenates for each sample ensures that each sample occupies an equal “slice” of the NGS analytical channel, such that the samples with high sperm counts do not swamp out those with low sperm counts. In this way, another marketable embodiment of the present inventive work would be quality-controlled 96, 384, or higher-well plates, within the wells of which are a constant number of moles of dried-down anti-human sperm antibody-polynucleotide concatenates, ready for cellular sample addition. Without the present inventive work, without expensive and laborious cell counting and normalization between samples, the background/related art method of multiplexing would likely produce data for some contributors of some samples rather than each contributor to each sample. The added labor of cell counting would vastly reduce throughput and increase costs.

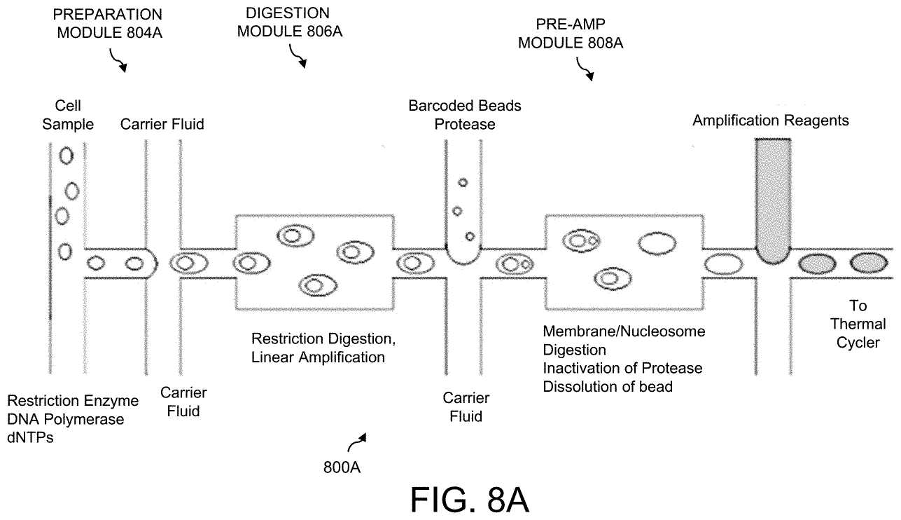

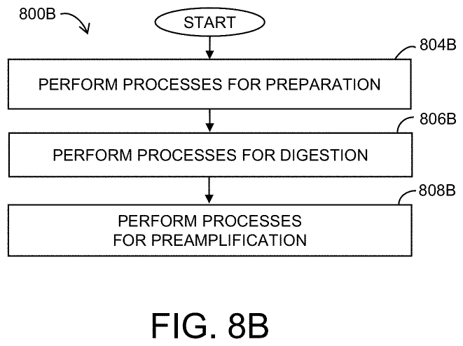

In FIG. 8A, a microfluidic device 800A may be provided for enhanced single cell or nucleus next generation sequencing according to some implementations of the present disclosure. The steps comprising the molecular biology workflow shown in FIG. 8A may be divided into three main modules associated with microfluidic device 800A, namely, a preparation module 804A, a digestion module 806A, and a pre-amplification module 808A. With respect to preparation module 804A, cells may be placed within microdroplet emulsions along with enzymes needed for polynucleotide concatenate digestion and converted into single strands. With respect to digestion module 806A, proteinase and bead bound primers may be added and the primers liberated from the beads. With respect to pre-amplification module 808A, the proteinase may be de-activated via thermal or other denaturation, and reagents and enzymes may be added for subsequent amplification from the genomic DNA.

To carry out these three modules, what is described is a three-step microfluidic process with respect to microfluidic device 800A of FIG. 8A, and with a flowchart 800B of FIG. 8B. Microfluidic device 800A may enable each of the modules to carry out the functions sequentially. With preparation module 804A, a process for preparation is performed (step 804B of FIG. 8B), which may include the generation of emulsions of solution within an immiscible carrier fluid, containing single cells with reagents and enzymes needed for carrying out a first set of molecular biology reactions, and the transition of the cells into an incubation chamber within which molecular biology reaction(s) take place. Next, with respect to digestion module 806A, a process for preparation is digestion is performed (step 806B of FIG. 8B), which may include the subsequent merging of the emulsions with a second set of reagents and/or enzymes, and transition to a second incubation chamber within which a second set of molecular biology reaction(s) takes place. Then, with respect to pre-amplification module 808A, a process for pre-amplification is performed (step 808B of FIG. 8B), which may include the subsequent merging of the emulsions with a third set of reagents and/or enzymes, and creating emulsions suitable for polymerase chain reaction in preparation for NGS sequencing.

As described, the foregoing methods rely on “positive skimming”, where an agent binds to and selects the cells that will proceed to be sequenced. In some situations, the process may be better served with use of “negative skimming,” where an agent is utilized to bind to the cells that will be removed and not proceed to sequencing. For example, if an entire collection of tumor cell types is desired to be read from a tumor sample, and the cell surface markers of these types is not known a-priori, biotinylated antibodies may be used that recognize normal epithelium, endothelium, immune cells, etc., bound to a streptavidin-coated bead, and removed from the sample using a magnet. The sub-sample of cells left behind would thus not need to be bound by a separate agent linked to a polynucleotide concatenate. Instead of the polynucleotide concatenates, simple oligonucleotides may be used that contain the sample-identifying barcodes and either the 5′ primers or the 3′ primers for the target loci, as partners for those that will be provided on the bead. Because the sample is a sub-sample after “negative skimming,” these primers may be supplied in free solution form, and no longer do they need to be bound to an agent to positively select cells for proceeding; all of the cells of the sub-sample may proceed to contribute to the SCNGS data without consuming too much of the channel bandwidth of the cartridge(s).

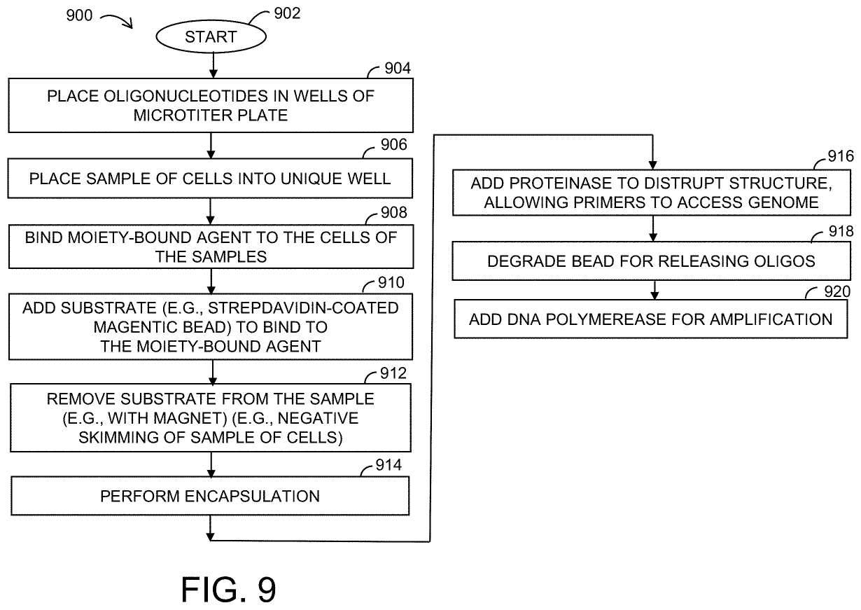

FIG. 9 is a flowchart 900 for describing an enhanced method of single cell or nucleus next generation sequencing according to some implementations of the present disclosure. The method pertains to the case of “negative skimming” as described above, where the following steps may be utilized, although the order may be varied. Note that the method described here may or may not utilize related or similar steps or techniques as described in relation to the above-described techniques.

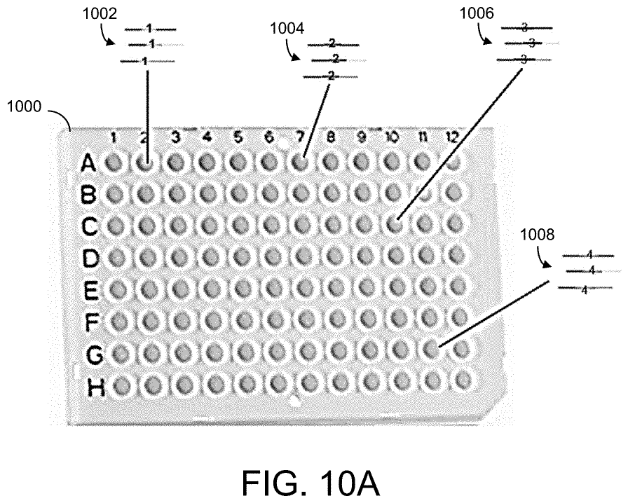

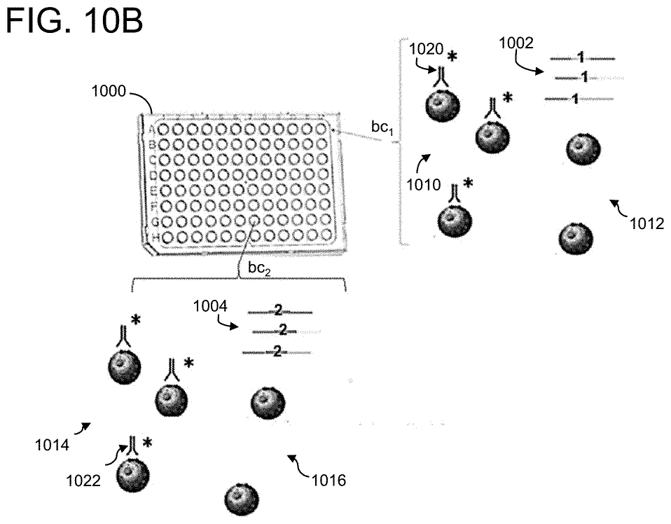

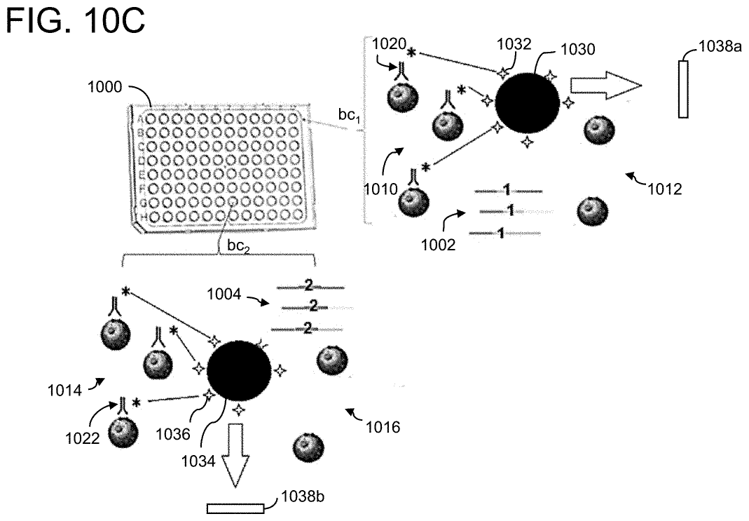

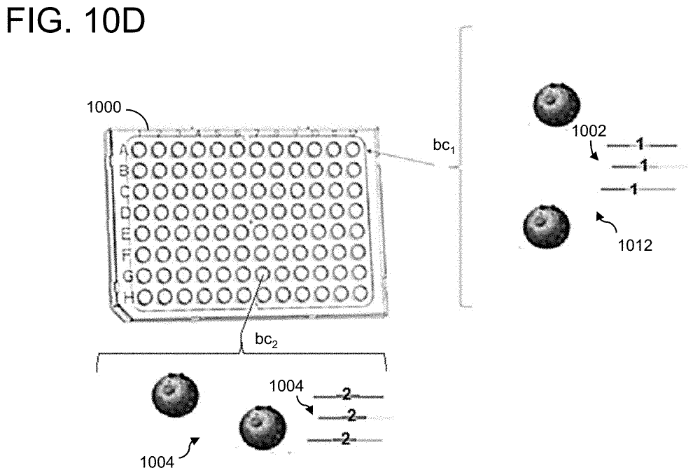

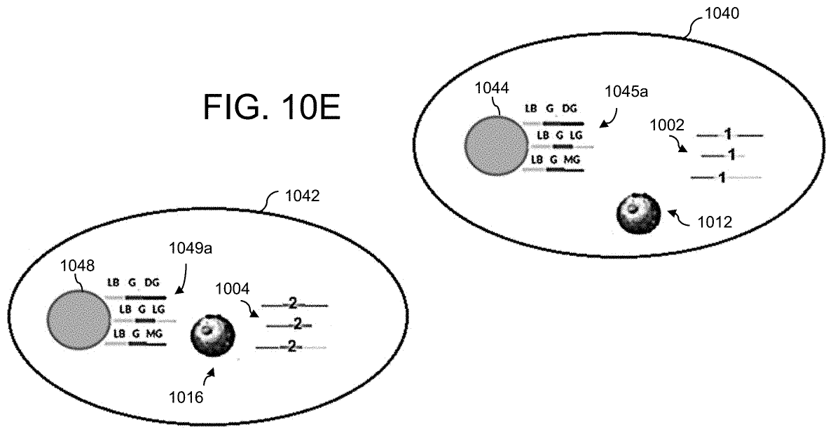

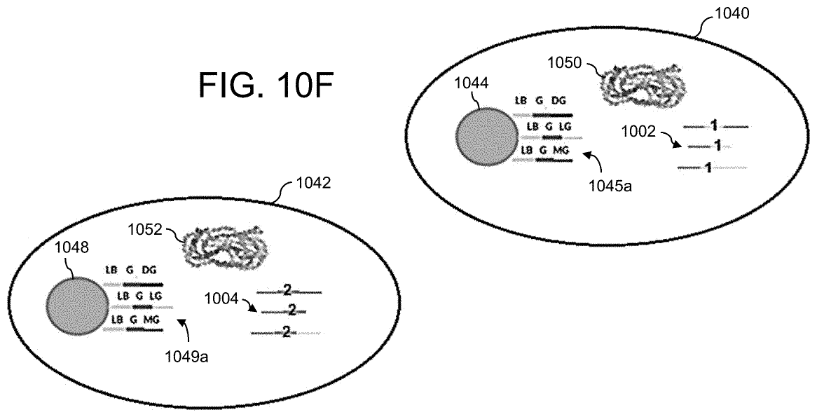

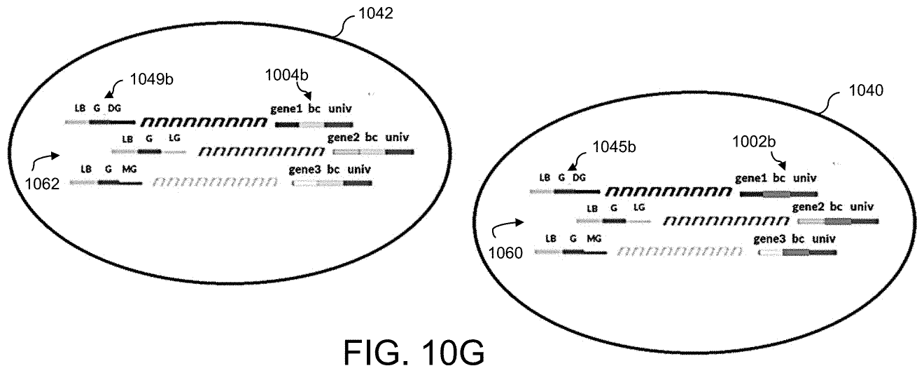

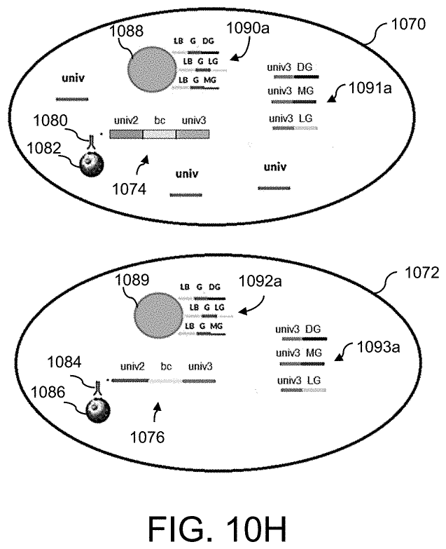

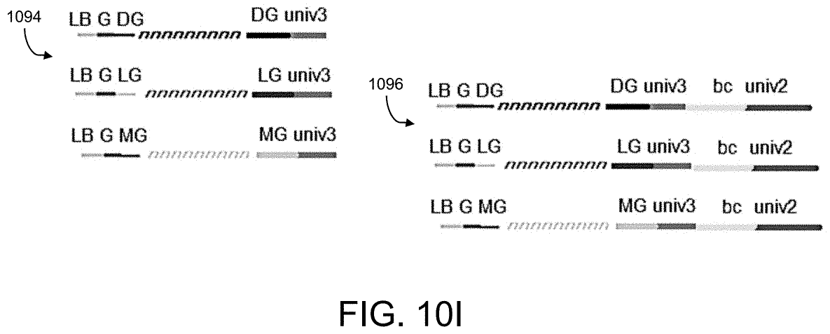

Beginning at a start block 902 of FIG. 9, a plate of oligonucleotides in solution form or dried-down may be provided, such that each well of the plate has the same “univ” and “gene” sequences but different barcodes (step 904 of FIG. 9). In FIG. 10A, what is shown is an example illustrative representation of a microtiter plate 1000, where a set of oligonucleotides 1002 (associated with barcode “1”) is provided in a first well, a set of oligonucleotides 1004 (associated with barcode “2”) is provided in a second well, a set of oligonucleotides 1006 (associated with barcode “3”) is provided in a third well, and a set of oligonucleotides 1008 (associated with barcode “4”) is provided in a fourth well.

Samples of cells may be placed in each well (step 906 of FIG. 9). A moiety-bound agent may be bound to the cells of the samples (step 908 of FIG. 9). The agent may recognize epitopes or other agents on the surface of the cells that are intended to be eliminated from the sample. In one example, biotin may be utilized as the moiety and the epitope may be one expressed on normal cells, thereby leaving, for example, tumor cells of a tumor sample unbound. In FIG. 10B, what is shown is an example illustrative representation of cells 1010 of a first sample provided in the first well with the set of oligonucleotides 1002 (associated with barcode “1”), and also cells 1014 of a second sample provided in the second well with the set of oligonucleotides 1004 (associated with barcode “2”). As shown, a moiety-bound agent 102 may be bound to some of cells 1010 of the first sample, but not to other cells 1012 of the first sample. Similarly, a moiety-bound agent 1022 may be bound to some of cells 1014 of the second sample, but not to other cells 1016 of the second sample.

A substrate, such as a streptavidin-coated magnetic bead, may be added to bind to the moiety-bound agent (step 910 of FIG. 9) and then removed from the sample, for example, with use of a magnet (step 912 of FIG. 9). In FIG. 10C, what is shown is an example illustrative representation of a magnetic bead 1030 having a streptavidin-coat 1032 to bind to moiety-bound agent 1020 of a cell of the first sample, where a magnet 1038a is used to remove cells 1010 of the first sample that are bound to magnetic bead 1030, leaving other cells 1012 remaining. Similarly, what is shown is a magnetic bead 1034 having a streptavidin-coat 1036 to bind to moiety-bound agent 1022 of a cell of the second sample, where a magnet 1038b is used to remove cells 1014 of the second sample that are bound to magnetic bead 1034, leaving other cells 1012 remaining. This leaves negatively-skimmed samples of cells. In FIG. 10D, what is shown is an example illustrative representation of other cells 1012 of the first sample remaining together with the set of oligonucleotides 1002 (associated with barcode “1”), and other cells 1004 of the second sample remaining together with the set of oligonucleotides 1004 (associated with barcode “2”).

The cells may be encapsulated into microdroplet emulsions along with beads bound to univ-bc-gene primers as described previously (step 914 of FIG. 9). In FIG. 10E, what is shown is an example illustrative representation of a microdroplet 1040 which encapsulates other cells 1012 of the first sample, the set of oligonucleotides 1002 (associated with barcode “1”), and a bead 1044 that is bound with univ-bc-gene primers 1045a. Similarly, what is shown is of a microdroplet 1042 which encapsulates other cells 1016 of the second sample, the set of oligonucleotides 1004 (associated with barcode “2”), and a bead 1048 that is bound with univ-bc-gene primers 1049a.

Next, proteinase may be added to the microdroplets for disrupting of the nucleosome/DNA structures (e.g., as well as the chromatin), thereby allowing access to the genome (step 916 of FIG. 9). Here, the nuclear membrane may be disrupted, either osmotically and/or with assistance of the proteinase, and the nucleosomal proteins may be digested, releasing the genomic DNA. In FIG. 10F, what is shown is an example illustrative representation of a proteinase 1050 which is added to microdroplet 1040, which disrupts the nucleosome/DNA structures for releasing the genomic DNA, and a proteinase 1052 which is added to microdroplet 1042, which disrupts the nucleosome/DNA structures for releasing the genomic DNA.

The bead may then be degraded or dissolved, for releasing the oligos or primers (step 918 of FIG. 9). A DNA polymerase may then be added, and targeted PCR is carried out for amplifying the target gene panel (step 920 of FIG. 9). In some implementations, genomic amplification may be performed according to standard practice. Amplification results in amplification products that are labelled with barcode tags indicative of both the cell of origin at one end, and the sample of origin at the other end. In FIG. 10G, what is shown is an example illustrative representation of amplification products (e.g., amplification products 1060 in microdroplet 1040 and amplification products 1062 in microdroplet 1042) which are the result of the primers being used with PCR on the released DNA or cDNA. With respect to microdroplet 1040, bead primers 1045b may constitute the 5′ set of primers for the target gene panel, whereas univ-bc-gene strands 1002b may constitute the 3′ set of primers for the target gene panel. Amplification products 1060 contain barcodes indicative of the cell of origin at their 5′ end, and indicative of the sample of origin at their 3′ end. Similarly, with respect to microdroplet 1042, bead primers 1049b may constitute the 5′ set of primers for the target gene panel, whereas univ-bc-gene strands 1004b may constitute the 3′ set of primers for the target gene panel. Amplification products 1062 contain barcodes indicative of the cell of origin at their 5′ end, and indicative of the sample of origin at their 3′ end.

Thus, what are produced are amplification products where the polynucleotide barcode integrated into the primer at the 3′ end (or 5′ end) informs as to the sample (e.g., microtiter plate well) and the bead library barcode integrated into the primer present at the 5′ end (or 3′ end) informs as to the cell within that sample. Notably, the amplification products of the sample multiplex are obtained from only a subset of cells in each sample.

Because of the reduction in the number of molecular biology steps, if used with a targeted DNA or genomic sequencing, such a negative skimming procedure can be used with a two-step microfluidics device such as those previously described in the background/related art (see, e.g., U.S. Pat. No. 10,501,739 to Eastburn) and if used with an RNA transcriptome, a one-step microfluidics device may be employed (see, e.g., U.S. Pat. No. 10,752,895 to Church et al.).

The foregoing therefore describes “negative skimming” and “positive skimming”. The “negative skimming” approach may be utilized such that no skimming takes place at all, resulting in all cells of each sample contributing to NGS reads, consuming more channel bandwidth. This would result in lower-order multiplexing than the skimming methods, because each sample would take up more of the channel bandwidth. In this case, however, the procedure is the same as steps 904 through 920 of FIG. 9 (with FIGS. 10A through 10G), except that steps 908, 910, and 912 of FIG. 9 (with FIGS. 10B and 10C) are omitted. Again, if used with targeted DNA or genomic sequencing, this procedure may be employed with a two-step microfluidics device, such as those previously described in the background/related art (see, e.g., U.S. Pat. No. 10,501,739 to Eastburn) and if used with an RNA transcriptome, a 1-step microfluidics device may be employed (see, e.g., U.S. Pat. No. 10,752,895 to Church et al.).

At least in some cases, however, both the negative and the non-skimming approaches of the present disclosure may suffer from the same limitations as the current state-of-the-art multiplexing approaches with SCNGS. In particular, the limitations of the acceptance and passing of cells to amplification and NGS, where some samples may potentially contribute far too many numbers of cells to the bandwidth than others, making it prudent to limit the multiplex factor. Without use of an agent to bind the cells selected to “pass” to amplification and NGS, there may be no suitable means to normalize them without laborious and time-consuming cell counting and dilution methods. Therefore, this approach may not be implemented in the type of high-throughput types of environments that are desired.

In some implementations, the objectives of the present disclosure may be achieved with use of other agent-bound polynucleotides. For example, instead of a polynucleotide concatenate, a single element of barcode and universal sequences (“univ2-bc-univ3”) may be utilized to tag the cells, if the universal sequence (“univ3”) is also present at the 5′ end of one half of the primers used to amplify target sequences. If the beads contribute the 5′ primers, each sample is apportioned to a well of a microtiter plate, then the 3′ primers without any barcode sequence may be contributed in bulk across all samples/wells. If the agent-bound “univ2-bc-univ3” elements are apportioned to each well in a manner such that each well gets an agent-bound element with a different barcode bc sequence, then once this agent binds the cells and these primers and cells are encapsulated into a microdroplet (see, e.g., FIG. 10H) and the necessary steps accomplished including liberating the primers, amplification may take place for producing two types of amplicons.

In FIG. 10H, what is shown is an example illustrative representation of a microdroplet 1070 which encapsulates univ3-gene elements 1091a (3′ primers), a bead 1088 with its oligios 1090a (5′ primers), and an univ2-bc-univ3 element 1074 with an agent 1080 that binds to the epitope of a cell 1082. In addition, FIG. 10H shows a microdroplet 1072 which encapsulates univ3-gene elements 1093a (3′ primers), a bead 1089 with its oligios 1092a (5′ primers), and an univ2-bc-univ3 element 1076 with an agent 1084 that binds to the epitope of a cell 1086.

In FIG. 10I, the two types of amplicons from amplification may be cell-tagged amplicons 1094 that have not incorporated the element and are not sample tagged, and cell-tagged amplicons 1096 that have incorporated the element and are sample tagged.

Unlike the polynucleotide concatenate method, this method only incorporates some of its amplicons with the sample tag, and due to the stochastic nature of polymerase chain reaction, the proportion of tagged to not-tagged will be different from cell to cell. To avoid not-tagged amplicons from consuming NGS channel space at the expense of tagged, another amplification step could be performed using the universal sequence on the first set of primers linked to the beads (see FIGS. 10H and 10I—LB) and the universal sequence linked to the agent-bound element (see FIGS. 10H and 10I—univ2).

The advantage of this approach is that, instead of partitioning agent-bound long concatenates with unique barcode sequences to wells of a plate, which would require 96 or 384 different long concatenates, agent-bound short elements may be apportioned to each well of a plate where each element contains a unique barcode. That is, one can save on expenses since 96 or 384 different short sequences are needed in contrast to long ones.

On the other hand, the disadvantage of this approach compared to the polynucleotide concatenate approach could be significant. Because the ratio of tagged to not-tagged varies, often substantially, from cell to cell, we may introduce an uncontrollable form of bias into the results. Here, the ratio of tagged to not-tagged target gene amplicon levels will vary from cell to cell, cellular proportions for certain unknowable gene targets may be unreliable, and after normalization PCR (designed to make the mass of DNA from each sample the same), some cells will “swamp out” other cells/samples and consume disproportionate amount of NGS cartridge channel space. This would provide misleading results on the existence and proportions of cell types in the samples. In contrast, with the polynucleotide concatenate method, every target sequence that is amplified contains a cell and sample tag from the beginning, and therefore the opportunity for this type of amplification bias and misleading result may be avoided.

Swab aspects of the present disclosure are now described. With respect to the background/related art, forensic casework most always uses cotton swabs. With cotton swabs there are often problems removing biological material from the cotton matrix; as the cotton swab dries after collection, the biological material can adhere to the swab. For example, due to the saccharic composition of the spermatocyte membrane, spermatocytes stick to solid supports, especially cotton. See, e.g., Lazzarino, M. F. et al., “DNA Recovery from Semen Swabs with the DNA IQ System,” 2008, Forensic Science Communications 10(1). In order to release the maximum amount of material from the swabs, a variety of buffers have been tested and compared to the standard differential extraction buffer. Use of detergents such as 1-2% sodium dodecyl sulfate (SDS) has shown to increase sperm cell or nucleus recovery (see, e.g., Norris, J. V. et al., “Expedited, chemically enhanced sperm cell or nucleus recovery from cotton swabs for rape kit analysis,” 2007, J Forensic Sci 52(4): 800-5) as well as the recovery of other cell or nucleus types but still, a not insignificant fraction of cases involve low levels of cellular material to begin with (see, e.g., “touch DNA” cases), and these often produce DNA results that are below the thresholds for reliable interpretation. Thus, it is important to maximize the release of cellular and/or genetic material from swabs prior to SCNGS analysis. Previous patent disclosures for nucleic acid purification have described this challenge and referenced the first uses of cellulase for recovery of genetic material from dried cotton swabs (see, e.g., U.S. Pat. No. 10,464,065 to Selden et al.,) and the addition of low amounts of cellulase has shown to release more epithelial and sperm cells from the cotton swab matrix than buffer elution alone. See, e.g., Voorhees, J. C. et al., “Enhanced elution of sperm from cotton swabs via enzymatic digestion for rape kit analysis.” 2006, J Forensic Sci 51(3): 574-9.

In addition to the use of cellulase, to facilitate the collection of discrete cells from swabs re-hydrated after desiccation, antiagglutinins such as antibodies can be used to prevent clumping of cells after they are released from the cellulose matrix. Various antiagglutinins are commercially available for this purpose, such as Goat Anti-PNA antiagglutinin of Vector Laboratories, and products are available for use with particular cell or nucleus types such as blood cells (anti-hemagglutinin).

Cellulase has not become an integral part of swab preparation protocols in the forensic science community. This is largely because the effects are modest, because the original descriptions used exocellulases, and were overly simplistic in the interpretation. Cellulases come in two varieties—exocellulases and endocellulases. For disrupting cotton swabs, which are made almost entirely of cellulose (see, e.g., entry for “Cotton” at Wikipedia) digestion at sites between the ends would be more valuable than digestion from the ends, primarily because there are far more of these sites.

Polyester swabs are also used in forensic science, but with the presently disclosed SCNGS inventive work, its likelihood of disruption of the current state-of-the-art in forensic and diagnostic science, and adoption, the relevance for complete release of cells and/or their nuclei from desiccated swabs as a precursor for SCNGS, it is likely that the present inventive work could contribute towards a new FBI and/or American National Standards Institute (ANSI) Quality Assurance Standard mandating cellulose swabs for the future in forensic science (with implications for analogous Clinical Laboratory Improvement Amendments or “CLIA” standards in diagnostics).

Here, what is described is the use of endocellulases for the extraction of cells and/or nuclei from, and preparation of dried forensic cotton swabs for horizontal SCNGS, with or without the use of lysis buffers that facilitate the action of the endonuclease, by enabling the release of intact cells or nuclei from within the swabs (such as those containing proteinase K, and/or hypotonic solutions/buffers). What is also described is the use of endo-cellulases for the digestion of cellulose in cotton swabs and release of cellular material for use in SCNGS workflows. What is further disclosed is a combination of cellulase and anti-agglutinin tailored to specific types of evidence swabs, such as buccal or blood swabs.

Accordingly, as described herein, the present inventive work was developed to effect a disruptive transformation of forensic genetics and diagnostics by enabling human identity and/or diagnostic profiles from individual contributors present in sample mixtures. At least some aspects of the present inventive work involve a “horizontal” approach to a recently introduced method of isolating and examining single cells in massively parallel formats, called SCNGS, as opposed to the “vertical” approach used today. In so doing, the present inventive work may enable larger numbers of samples to be analyzed for fewer numbers of cells per sample, reducing cost points such that forensic genetics and diagnostics applications of SCNGS become practical. Notably, the present inventive work may help solve, once and for all, the “mixture problem” which has plagued the forensic genetics and human identity market. In so doing, the present inventive work could represent an inflection point in the state-of-the-art in forensics and diagnostics as a fundamental, transformative disruption that brought about the effective, efficient and cost-compatible application of SCNGS to these fields.

### Definitions

In some implementations of the present disclosure, the following definitions may apply in relation to the above-described processes.

The term “nucleotide” is meant to convey any string of nucleosides forming any sequence, whether of many nucleosides (“oligonucleotide”) or a large number (“polynucleotides”). A “nucleoside” is a 2′-deoxy and/or 2′-hydroxyl form of a nucleic acid building block, whether naturally occurring or synthetic, the latter of which are commonly referred to as “analogs”. A nucleoside consists of a nitrogenous base covalently attached to a sugar (ribose or deoxyribose) but without the phosphate group. A nucleotide on the other hand consists of a nitrogenous base covalently attached to a sugar (ribose or deoxyribose) and from one to three phosphate groups. See, e.g., Alberts B, Johnson A, Lewis J, Morgan D, Raff M, Roberts, K, Walter P., “Molecular Biology of the Cell or Nucleus,” Sixth Edition, W. W. Norton & Company. The present inventive text considers the terms nucleosides, nucleotides, and analogues thereof as equivalent, represented as G, A, T, C or dG, dA, dT or dC, as long as they hybridize specifically to their cognate nucleotide, nucleoside, analog where G hybridizes with C and vice-versa, and A hybridizes with T and vice versa and will hereafter be referred to as “nucleotides”. Nucleotides may come in a 2′-deoxy or a 2′-hydroxyl forms where they are referred to as deoxynucleotide or a nucleotide, respectively. See, e.g., U.S. Pat. No. 10,501,739 to Eastburn, and U.S. Pat. No. 10,752,895 to Church et al. Examples of nucleotide analogues are given by Scheit. See, e.g., Scheit, K., “Nucleotide Analogues, Synthesis and Biological Function,” John Wiley, New York, 1980. Polynucleotides thereof with enhanced hybridization characteristics described by Uhlman and Peyman. See, e.g., Uhlmann E and Peyman A., “Antisense Oligonucleotides: A New Therapeutic Principle,” 1990, Chemical Reviews (90)4: 544. Modified nucleotides may include, for example diaminopurine, 5-fluorouracil, 5-bromouracil, . . . , etc. Nucleotides are polymerized into nucleic acid molecules via the process of DNA replication, which is the process referred to when using the terms “amplification” or “polymerization” and is extensively taught in various textbooks such as Kornberg 2006. See, e.g., Kornberg A., “DNA Replication,” 1980, WH Freeman & Co Ltd.; and Watson J, Baker T, Bell S, Gann A, Levine M, Losick R, “Molecular Biology of the Gene,” 7^{th} Edition, 2014, Pearson Publishers.

“Oligonucleotides” as used herein refer to single-stranded nucleic acid molecules comprised of a string or series of from 5 to 10, 15, 20 up to 100 nucleotide bases linked in a 5′ to 3′ orientation. A “polynucleotide” is a set of linked oligonucleotides, and/or a string of single-stranded nucleic acid molecules comprised of a string or series of from 100, 200, 1000, 2000 to 10,000 nucleotide bases linked in a 5′ to 3′ orientation.

The notations 5′ and 3′ are used to express one end versus the other end of target amplicons, and can be used interchangeably. For example, if the vendor “bead library” provides amplification primers for one end of an amplicon, the “antibody-concatenate” would provide amplification primers for the other end whether the first end is called 5′ or 3′, and whether the corresponding second end is called 3′ or 5′.

“Complimentary” or “substantially complementary” nucleotides are defined as those for which at least 80% or more of the nucleotides, usually 90 or 95% or more, appositionally overlap when iteratively compared, nucleotide by nucleotide along the length of at least one of the nucleotides, such that the nucleotides are capable of base-pairing or hybridizing to form a duplex of double-stranded DNA under any of a variety of salt and/or buffer conditions. Assessment of complementarity or substantial complementarity is accomplished via either visual registration of the sequences in various overlap configurations and/or with computer devices such as with the use of programs, written in any language, for example, Python programming language with the “Zip” function with lists, tuples or arrays, embedded or not in dictionaries, comprised of iterations of at least one sequence starting with one nucleotide to define a stretch or length of sequence, and progressing along the sequence, re-adjusting the alignment phase by one nucleotide until the end is reached.

The term “hybridization” refers to the non-covalent union of two single-stranded nucleotide sequences based on sequence complementarity, such that they anneal or non-covalently bind together to form a duplex of double-stranded DNA. This union may be accomplished at low (<100 mM) or high (>100 mM) salt concentrations, whether buffered or not, usually below 1M salt concentrations, typically below 500, 200 or 100 mM salt concentrations and are most often carried out under rigorous or stringent binding conditions such that sequence complementarity or substantial complementarity is required, and thus is at least substantially sequence-dependent. Longer oligo/polynucleotide complements require lower annealing temperatures than shorter complements, as do those of greater GC content, and sequence-specific, and therefore meaningful hybridization is typically carried out at temperatures of at least 22 degrees Celsius, to 30, 37, 40, up to 90 degrees Celsius as described fully in Sambrook et al. See, e.g., Sambrook, Fritsche and Maniatis, “Molecular Cloning: A Laboratory Manual,” 2^{nd} Edition, 1989, Cold Spring Harbor Press. When used, the term “hybridization” is intended to indicate the union, binding, annealing, of single-stranded nucleotide/oligonucleotide/polynucleotide sequences as a function of their sequence complementarity as complementary nucleotides under reasonably achievable solution conditions, and for this reason is considered a “sequence specific” event.

As used herein the term “barcode” indicates any nucleotide sequence used to unambiguously identify an oligonucleotide or polynucleotide sequence, or set of sequences, in which it is embedded or attached. The sequence may be randomly or non-randomly generated, but in either case must be long enough (n) such that its occurrence in the larger oligo/polynucleotide in which it is embedded or attached is not expected by chance within it as a function of the polynomial expansion of n elements given an equal or even a biased likelihood of occurrence of any of the four nucleotide types in any single position within the oligo/polynucleotide. The barcode may be a few, a dozen, or a few dozen nucleotides in length (e.g., from 5, 10, 15, 20, . . . , to about 50 or 75 or more nucleotides long). It is composed of dA, dT, dG and dC nucleotides in any combination or order, as well as any nucleotide derivatives or analogues thereof and in the context of the present inventive work, represents an intersection of the elements comprising the set of gene-specific nucleotide containing oligo/polynucleotides attached to a given, discrete bead. Barcode sequences are essentially non-complimentary, representing a minimally cross-hybridizing set. Their annealing temperatures (Tₘ) may be from 20 degrees Celsius, to 25, to 30, 35, 40, 45, 50, 55, 60, 65 up to about 90 degrees Celsius and each is defined by a unique sequence-dependent Tₘ.

“Beads” are nano or micrometer sized spheres that are capable of adsorbing to, or otherwise binding to oligonucleotides and/or polynucleotides. In certain embodiments the bead has a magnetic core or cortex, and may be functionalized with a moiety or a protein such as streptavidin which binds to moieties such as biotin often incorporated at the 5′ end of oligo/polynucleotides. Examples include Dynabeads from ThermoFisher Scientific (see, e.g., Immunoprecipitation Dynabeads Products, Thermo Fisher Scientific—US) which are available in various sizes and configurations such as Streptavidin coated M-280 (current gold standard for isolation of biotinylated nucleic acids) (see, e.g., Magnetic Beads: Life Science Applications; e.g., news-medical.net), M-270, MyOne C1, DynaMag-2, and Pierce tradenames, AMSBIO's MagSi magnetic beads, IBA's MagStrep Type3 XT, which most often are magnetic allowing for convenient purification from cellular debris, reaction salts and buffers as reviewed in Bosnes et al., 2013. See, e.g., Bosnes M, Deggerdal A, Rian A, Korsnes L, Larsen F., “Magnetic Separation in Molecular Biology,” In: Hafeli U, Schutt W, Teller J, Zborowski M, editors. “Scientific and Clinical Applications of Magnetic Carriers,” Springer Science & Business Media, 2013, pp. 269-286. The term “bead” is meant to be taken as equivalent to any other solid substrate including slides, beads, chips, particles, strands, gels, sheets, tubing, spheres, containers, capillaries, pads, slices, films, dishes, plates in any configuration 96-well, 28 well 384 well etc. made of any substance such as but not limited to paramagnetic materials, ceramic, plastic, glass, polystyrene, methylstyrene, acrylic polymers, titanium, any other metal, latex, sepharose, cellulose, nylon etc., such that might enable sequestration of multiplex oligo/polynucleotide primer sets with specific cells. The term “attach” is meant to mean the covalent sharing of electrons between atoms of two specific molecules, where one of the molecules is part of a solid substrate or a bead and the two can be said to be “bound”. Beads may be purified using any means, such as magnetic separation or centrifugation for example, and the nucleic acids bound or attached to the beads can be purified using any method for the purification of beads or nucleic acids bound to beads including via use of solid DNA binding substrates or old-school extraction and precipitation. See, e.g., Sambrook et al., “Molecular Cloning: A Laboratory Manual,” 4^{th} Edition, 2012, CSHL Press).

“Multiplexing” refers to the targeting of a set of unrelated, unlinked genetic positions, “loci”, or “genes”, during execution of a detection method, such as polymerase chain reaction or “amplification”. Multiplexing can take place inside of a cell or nucleus or outside of a cell, inside of a micro or nanometer sized reaction vessel or outside of one, or most commonly, in a reaction tube and can be carried out using primers in solution or bound to a solid substrate, such as a slide, plate, bead, semiconductor etc. Typically multiplexing is carried out by, instead of supplying a single 5′/3′ primer pair targeting a single genetic locus, providing a set or collection of 5′/3′ primer pairs during amplification set-up. “Amplification” means polymerase chain reaction (PCR) (see, e.g., Mullis et al., 1986, Cold Spring Harb Symp Quant Biol 51 Pt 1:263) and may include the use of gripped or anchored primers as in the case of “grip PCR”, or incorporate PCR in various schema for specific amplification purposes such as RACE PCR, Ligation Chain Reaction PCR, standard or nested PCR, real-time PCR and/or multiplexed PCR.

The term “sequencing” is meant to convey any method of sequence detection—such as, Sanger sequencing, sequencing by synthesis on solid substrate, or n solution, sequencing by hybridization (SBH), sequencing by ligation (SBL), TaqMan reporter probe digestion, pyrosequencing, etc.

The term “nanodroplet”, “vessel” and “reactor” are intended to signify the same meaning in the context of the present inventive work, indicating a micro or nanometer sized vesicle, or sphere which may contain a bead with nucleic acids attached to it, and may contain a cell, within which biochemical and molecular biology reactions take place including DNA replication, polymerization, amplification etc., such that the reactions are isolated from those taking place in other nanodroplets which are similarly isolated. By “isolated” it is meant that the nucleic acid oligo/polynucleotides in separate nanodroplets are unable to hybridize with those from another.

Thus, enhancements to single cell or nucleus next generation sequencing have been described herein. What are now described are the various approaches as presented above and herein.

In one illustrative example, a method of the present disclosure may involve obtaining a bead library which includes a bead coupled to a plurality of oligonucleotides, the plurality of oligonucleotides comprising a plurality of first primers of a set of primer pairs for a targeted set of loci, each oligonucleotide further including a first barcode sequence that is common to the plurality of oligonucleotides on the bead; binding a cell or nucleus of a sample to a polynucleotide concatenate via a binding agent, the polynucleotide concatenate comprising repeating units of univ-bc-gene sequences, represented as univ-bc-gene1, univ-bc-gene2, . . . , to univ-bc-geneN, wherein the univ represents a universal sequence, the be represents a second barcode sequence that is common to the repeating units of univ-bc-gene sequences, and the gene1, gene2, . . . , to geneN comprise a respective plurality of second primers of the set of primer pairs for the targeted set of loci, and wherein N represents the number of loci in the targeted set of loci; wherein the univ and the gene are selected at each gene-univ junction of the univ-bc-gene sequences so as to create a restriction enzyme binding site at each gene-univ junction; and encapsulating, into a microvesicle, the bead coupled to the plurality of oligonucleotides and the cell or nucleus of the sample that is bound to the polynucleotide concatenate.

In some implementations of the above-described method, the method may further involve adding restriction endonuclease to the microvesicle for digestion, for separating the univ-bc-gene sequences from the polynucleotide concatenate into a plurality of individual univ-bc-gene units; causing a membrane of the cell or nucleus to be digested in the microvesicle, for primer access to a genome of the cell or nucleus; causing the bead to be degraded or digested in the microvesicle, for releasing the plurality of oligonucleotides from the bead; and performing a PCRprocess for amplifying regions of the genome based on the primer pairs for the targeted set of loci, which generates a plurality of amplicons each of which incorporates the first barcode sequence and the second barcode sequence; and wherein, for each amplicon, the first barcode sequence uniquely identifies the cell or the nucleus and the second barcode sequence uniquely identifies the sample.

In some implementations of the above-described method, binding the cell or the nucleus of the sample to the polynucleotide concatenate is performed by placing the polynucleotide concatenate into a well of a microtiter plate; placing the binding agent into the well of the microtiter plate, such that the binding agent binds to an end of the polynucleotide concatenate; and placing the sample of the cell or the nucleus into the well of the microtiter plate, so that the binding agent binds to the membrane of the cell or the nucleus of the sample, thereby binding the cell or the nucleus of the sample to polynucleotide concatenate. In some implementations of the method, where the polynucleotide concatenate comprises a single-stranded polynucleotide concatenate, the method may further involve converting the single-stranded polynucleotide concatenate into a double-stranded polynucleotide concatenate. In some implementations of the method, the steps of placing are repeated for each one of a plurality of samples of cells or nuclei that are placed into different wells of the microtiter plate, one sample per well, for binding the cells or the nuclei of each sample to a different polynucleotide concatenate, and the method further involves pooling together the different samples of the different cells or the nuclei; and performing microvesicle encapsulation after pooling together the different samples of the cells or the nuclei.

In some implementations of the above-described method, the binding agent is for binding to different types of cells of the sample for targeting a fraction of the cells of the sample; or the binding agent comprises an antibody which binds to an epitope of the membrane of the cell or nucleus of the sample; or the binding agent comprises an antibody which binds to an epitope that is differentially expressed amongst cells of the sample for targeting a fraction of the cells of the sample; or the binding agent comprises an antibody which binds to an epitope of the membrane of the cell or nucleus of the sample, the polynucleotide concatenate is provided with a moiety on its end, the moiety comprises biotin which binds to streptavidin, and the antibody comprises a streptavidin-conjugated antibody; or the bead library comprises a vendor bead library; or the first primers of the set of primer pairs comprise 3′ primers and the second primers of the set of primer pairs comprise 5′ primers; or the first primers of the set the primer pairs comprise 5′ primers and the second primers of the primer pairs comprise 3′ primers, or the targeted set of loci comprise CODIS STRs.

In another illustrative example, a method of the present disclosure may involve obtaining a bead library which includes a bead coupled to a plurality of oligonucleotides, the plurality of oligonucleotides comprising a plurality of first primers of a set of primer pairs for a targeted set of loci, each oligonucleotide further including a first barcode sequence that is common to the plurality of oligonucleotides on the bead but different from bead to bead; generating a plurality of polynucleotide concatenates based on at least the bead library, each polynucleotide concatenate comprising repeating units of univ-bc-gene sequences, represented as univ-bc-gene1, univ-bc-gene2, . . . , to univ-bc-geneN, wherein the univ represents a universal sequence, the be represents a second barcode sequence that is common to the repeating units of univ-bc-gene sequences in the polynucleotide concatenate but different from the other polynucleotide concatenates, and the gene1, gene2, . . . , to geneN comprise a respective plurality of second primers of the set of primer pairs for the targeted set of loci, and wherein N represents the number of loci in the targeted set of loci; wherein the univ and the gene are selected at each gene-univ junction of the univ-bc-gene sequences of the plurality of polynucleotide concatenates so as to create a restriction enzyme binding site at each gene-univ junction; and for each one of the plurality of polynucleotide concatenates, placing the polynucleotide concatenate into a respective unique one of a plurality of wells of a microtiter plate, and converting the polynucleotide concatenate into a double-stranded polynucleotide concatenate, thereby producing a plurality of double-stranded polynucleotide concatenates; placing a binding agent into the plurality of wells, such that the binding agent binds to ends of the plurality of double-stranded polynucleotide concatenates; for each one of a plurality of samples of cells or nuclei, placing the sample into the respective unique one of the plurality of wells, so that the binding agent binds to a membrane of the cells or the nuclei of the sample, thereby binding the cell or the nucleus of the sample to the double-stranded polynucleotide concatenate, for thereby producing the plurality of samples of cells or nuclei that are respectively bound to the plurality of double-stranded polynucleotide concatenates; and pooling together the plurality of samples of cells or nuclei that are respectively bound to the plurality of double-stranded polynucleotide concatenates. The method may further involve, for each one of a plurality of different beads associated with the bead library, encapsulating, into a microvesicle, the bead coupled to the plurality of oligonucleotides and the cell or nucleus that is bound to the polynucleotide concatenate; adding restriction endonuclease to the microvesicle for digestion, for separating the univ-bc-gene sequences from the polynucleotide concatenate into a plurality of individual univ-bc-gene units; causing a membrane of the cell or nucleus to be digested in the microvesicle, for primer access to a genome of the cell or nucleus; causing the bead to be degraded or digested in the microvesicle, for releasing the plurality of oligonucleotides from the bead; and performing a PCR process for amplifying regions of the genome based on the primer pairs for the targeted set of loci, which generates a plurality of amplicons each of which incorporates the first barcode sequence and the second barcode sequence; wherein, for each amplicon, the first barcode sequence uniquely identifies the cell or nuclei and the second barcode sequence uniquely identifies the sample.

In yet another illustrative example, a method of the present disclosure may involve obtaining a bead library which includes a bead coupled to a plurality of oligonucleotides, the plurality of oligonucleotides comprising a plurality of first primers of a set of primer pairs for a targeted set of loci, each first primer including a first barcode sequence that is common to the plurality of first primers on the bead; obtaining a plurality of second primers of the set of primer pairs for the targeted set of loci, in bulk, each being represented as univY-gene, wherein the univY represents a universal sequence Y and each gene represents one of the plurality of second primers; binding a cell or nucleus of a sample to a polynucleotide sequence via a binding agent, the polynucleotide sequence comprising a univX-bc-univY sequence, wherein the univX represents a universal sequence X, the be represents a second barcode sequence, the univY represents the universal sequence Y; encapsulating, into a microvesicle, the bead coupled to the plurality of oligonucleotides comprising the plurality of first primers of the set of primer pairs, the plurality of second primers of the set of primer pairs, and the cell or the nucleus of the sample that is bound to the polynucleotide sequence.

In some implementations of the above-described method, the method may further involve causing a membrane of the cell or nucleus to be digested in the microvesicle, for primer access to a genome of the cell or nucleus; causing the bead to be degraded or digested in the microvesicle, for releasing the plurality of oligonucleotides from the bead; and performing a PCR process for amplifying regions of the genome based on the primer pairs for the targeted set of loci, which generates a plurality of amplicons each of which incorporates the first barcode sequence and the second barcode sequence; wherein, for each one of at least some of the amplicons, the first barcode sequence uniquely identifies the cell or the nucleus and the second barcode sequence uniquely identifies the sample. In some implementations of the above-method, binding the cell or the nucleus of the sample to the polynucleotide concatenate is performed by placing the polynucleotide sequence into a well of a microtiter plate; placing the binding agent into the well of the microtiter plate, such that the binding agent binds to an end of the polynucleotide sequence; and placing the sample of the cell or the nucleus into the well of the microtiter plate, so that the binding agent binds to the membrane of the cell or the nucleus of the sample, thereby binding the cell or the nucleus of the sample to polynucleotide sequence. In some implementations of the above-method, the polynucleotide concatenate comprises a single-stranded polynucleotide concatenate, and the method further involves converting the single-stranded polynucleotide concatenate into a double-stranded polynucleotide concatenate. In some implementations of the above-method, the steps of placing are repeated for each one of a plurality of samples of cells or nuclei that are placed into different wells of the microtiter plate, one sample per well, for binding the cells or the nuclei of each sample to a different polynucleotide concatenate, and the method further involves pooling together the different samples of the different cells or the nuclei; and performing microvesicle encapsulation after pooling together the different samples of the cells or the nuclei.

In some implementations of the above-method, the binding agent is for binding to different types of cells of the sample for targeting a fraction of the cells of the sample; or the binding agent comprises an antibody which binds to an epitope of the membrane of the cell or nucleus of the sample; or the binding agent comprises an antibody which binds to an epitope that is differentially expressed amongst cells of the sample for targeting a fraction of the cells of the sample; or the binding agent comprises an antibody which binds to an epitope of the membrane of the cell or nucleus of the sample; or the polynucleotide sequence is provided with a moiety on its end, the moiety comprises biotin which binds to streptavidin, and the antibody comprises a streptavidin-conjugated antibody; or the bead library comprises a vendor bead library; or the first primers of the set the primer pairs comprise 5′ primers and the second primers of the primer pairs comprise 3′ primers; or the targeted set of loci comprise CODIS STRs.

In even yet another illustrative example, a process of the present disclosure (e.g., a three-step microfluidic process) is described for single cell next generation sequencing with use of a microfluidic device configured for enabling a generation of emulsions of solution within an immiscible carrier fluid, where the generation of the emulsions include single cells with a first set of reagents and/or enzymes desired for carrying out a set of molecular biology reactions, and where the process involves transitioning the single cells into a first incubation chamber within which a first set of molecular biology reactions take place; merging the emulsions with a second set of reagents and/or enzymes; transitioning to a second incubation chamber within which a second set of molecular biology reactions take place; and merging the emulsions with a third set of reagents and/or enzymes, for creating the emulsions for polymerase chain reaction in preparation for sequencing.

In some implementations of the above-described process, the solution is aqueous or non-aqueous, and wherein the multi-step process is for enabling multiplexed single cell next generation sequencing through tagging of the cells within a collection of samples. In some implementations of the above-described process, the process is for use with a simple set of antibody-bound or bead-bound barcode tags or types, and/or bead libraries, for enabling the tagging of cells within microreactors or vessels such that only a fraction of the single cells of the sample are tagged, which constitutes a skimming of cellular diversity from the sample. In some implementations of the above-described process, the first set of molecular biology reactions is restriction endonuclease digestion and/or linear amplification using a DNA polymerase. In some implementations of the above-described process, the second set of molecular biology reactions comprise proteinase digestion. In some implementations of the above-described process, the third set of molecular biology reactions enables geometric DNA amplification using a DNA polymerase.

In another illustrative example, a method of the present disclosure which is for multiplexed SCNGS may involve encapsulating a single nucleotide bound bead with a single cell or cell nucleus within a microvessel or on a solid substrate, within the context of a plurality of vessels or substrate binding sites, each containing a single bead and nucleus, where the cells or nuclei of a sample are tagged for sample identity by virtue of their being bound to an agent that contains a linked polynucleotide of repeating units in the form of a concatemer, where a restriction endonuclease binding site exists at each of the junctions between repeating units, and where within each vessel, the nucleotides are liberated from the beads and agent and extended from cDNA or genomic DNA from the cell via primer extension and/or polymerase chain reaction, to create polymerase chain amplicons such that the liberated bead bound primers and liberated cell tagging primers are both incorporated and suitable for next generation or massively parallel sequencing.

In some implementations of the above-described method, the nucleotide or genetic barcoding of the cells is contributed by the bead primers, and the nucleotide or genetic barcoding the sample is contributed by the agent bound nucleotides. In some implementations, the repeating unit is an oligonucleotide element of univ-bc-gene structure, where univ corresponds to a universal or common sequence, be corresponds to a specific barcode sequence, and gene corresponds to a nucleotide region of a gene. In some implementations, a second and/or a third universal binding site is incorporated at the 5′ and 3′ ends of the concatemer. In some implementations, the agent binds to all or substantially all of the cells of a sample. In some implementations, the agent binds to only a fraction of the cells of a sample. In some implementations, the preferred agent is an antibody that recognizes an epitope or binding site on the surface of the cells or nuclei.

In some implementations of the above-described method, the method uses a single or collection of polyclonal antibodies that recognize an epitope or binding site on the surface of the cells or nuclei. In some implementations, the method uses a single or collection of monoclonal antibodies that recognize an epitope or binding site on the surface of the cells or nuclei. In some implementations, the method uses a mixture of polyclonal and monoclonal antibodies that recognize an epitope or binding site on the surface of the cells or nuclei. In some implementations, the preferred agent is a liposome that merges or fuses indiscriminately with all of the cells or nuclei. In some implementations, the agent is a protein or mixture of proteins that binds to proteins, sugars or other entities on the surface of the cells or nuclei. In some implementations, the agent is a peptide or mixture of peptides that binds to proteins, sugars or other entities on the surface of the cells or nuclei. In some implementations, the polynucleotide concatenate contains a biotin at its 5′ end, and binds to the agent through strepavidin molecules linked to the agent. In some implementations, the agent bound nucleotides comprise a nucleotide containing one unit, or a polynucleotide constituting a concatemer containing repeating units or segments, of a first common or universal primer binding sequence, a unique barcode sequence, and a sequence common to a class of nucleotides, such as poly dT, and where a restriction endonuclease binding site exists at each of the junctions between repeating units.

In some implementations of the above-described method, the number of repeating univ-bc-gene units is 1, and one such element is bound to the agent. In some implementations, the number of repeating univ-bc-gene units is 2 through 12, and one such element is bound to the agent. In some implementations, the number of repeating univ-bc-gene units is 13 through N, and one such element is bound to the agent, where N is the maximum number of units that may be reasonably incorporated into a synthesized polynucleotide with state-of-the-art polynucleotide synthesis methods. In some implementations, the number of repeating univ-bc-gene units is 1, 2, or any number that is reasonably incorporated in a synthesized polynucleotide, and the units are bound to multiple sites on the agent. In some implementations, the number of repeating univ-bc-gene units is more than two dozen.

In some implementations of the above-described method, the repeating units are the same, targeting bulk cDNA, or the same genomic DNA sequence. In some implementations, the repeating units are different, targeting different cDNA or genomic DNA sequences. In some implementations, the concatenate is synthesized synthetically as a single polynucleotide. In some implementations, the first univ-bc-gene unit of the polynucleotide concatenate is a dummy unit that remains bound to the agent after restriction endonuclease digestion, and its gene sequence is not part of the gene sequence set needed for target loci amplification. In some implementations, additional universal or common sequences at the 5′ and 3′ ends of the polynucleotide concatenate are incorporated, enabling the concatenate to be amplified prior to restriction endonuclease digestion. In some implementations, the method involves libraries of a plurality of agent bound polynucleotides. In some implementations, the libraries of a plurality of agent bound polynucleotides are converted to double-stranded DNA.

In yet another illustrative example, what is claimed is a process of co-localizing an agent bound polynucleotide containing a single element or a concatenate of elements with beads coupled to other primers, and single cells within a micro or nanometer sized space, such as a micro or nanodroplet, well, or vesicle (“microreactor” or “nanodroplet”), such that the nucleotide element or elements that results from restriction digestion of the concatenate serve as half of the primers for DNA amplification (e.g., the 3′ primers) and the primers contributed by the bead represent the other half of the primers (e.g., the 5′ primers). In some implementations, what is further claimed is a process of digesting the polynucleotide, inside of a micro or nanometer sized space, bound to the agent or released from it, using a restriction endonuclease capable of binding to the restriction endonuclease binding sites present at each of the junctions between repeating units in the concatemer of 1. In some implementations, what is further claimed is a process of utilizing an organized distribution of barcoded primers linked to the agent for tracking the sample of origin for amplification products generated inside of emulsions, microvessels, microreactors etc to the sample of origin. In some implementations, what is further claimed is a process of utilizing a cipher to create barcodes linking amplification products generated inside of emulsions, microvessels, microreactors etc to the sample of origin. In some implementations, what is further claimed is a process of isolating such polynucleotide-linked, agent-bound cells in the preceding into micro or nanodroplets comprised of lipids or agarose.

In another illustrative example, what is claimed is a process of isolating such polynucleotide-linked, agent-bound cells into micro or nanodroplets comprised of droplets or wells, etched or not, into a solid 2-dimensional surface or substrate. In some implementations, what is further claimed is a process of co-localizing polynucleotide-linked, agent-bound cells into micro or nanometer sized spaces with single cells, captured through photon-assisted or electrokinetic gating. In some implementations, what is further claimed is a process where the primers constituted by the elements of the polynucleotides target specific human identity loci, such that each element contains a universal primer binding site, a discrete barcode and a set of gene specific primers or nucleotide class specific primer(s). In some implementations, what is claimed is further allowing a multiplexing of samples prior to integration of cells into the vessels, thereby reducing per sample costs for each SCNGS run. In some implementations, the method may be applied in the forensic sciences, or in routine diagnostics, or in R&D, or in Drug Discovery or Development.

In a further illustrative example, a method of the present disclosure for multiplexed SCNGS may involve encapsulating a single nucleotide bound bead with a single cell or cell nucleus within a microvessel or on a solid substrate, within the context of a plurality of vessels or substrate binding sites, each containing a single bead linked to one set of primers, and a single cell or nucleus, where the other set of primers creating pairs for each targeted DNA site is provided in bulk without any barcode, but contains a universal sequence at its 5′ end (univY-gene), where the cells or nuclei of a sample are tagged for sample identity by virtue of their being bound to an agent that contains a linked polynucleotide of a single unit in the form of a different universal, barcode and a universal region (univX-bc-univY), the nucleotides are liberated from the beads and agent and combined with bulk primers containing the partners extended from cDNA or genomic DNA from the cell via primer extension and/or polymerase chain reaction, to create polymerase chain amplicons, such that the liberated bead bound primers and liberated cell tagging primers are both incorporated and suitable for next generation or massively parallel sequencing.

In some implementations of the above-described method, the nucleotide or genetic barcoding of the cells is contributed by the bead primers, and the nucleotide or genetic barcoding the sample is contributed by the agent bound nucleotides. In some implementations, the primers containing nucleotide or genetic barcoding of the cells represent one half of the primer pair for each targeted sequence, the 3′ primers are provided in bulk and represent the other half of the primer pair for each targeted sequence, and the nucleotide or genetic barcoding the sample is contributed by the agent bound nucleotides. In some implementations, the agent bound polynucleotide contains, in order, a barcode sequence linked to a universal sequence, where the universal sequence is contained by each of the bulk primers at their 5′ ends. In some implementations, the agent bound polynucleotide contains, in order, a universal sequence linked to a barcode sequence linked to a universal sequence, where the universal sequence is contained by each of the bulk primers at their 5′ ends.

In some implementations of the above-described method, the polynucleotide is single-stranded. In some implementations, the polynucleotide is double-stranded. In some implementations, the agent binds to all or substantially all of the cells of a sample. In some implementations, the agent binds to only a fraction of the cells of a sample. In some implementations, the preferred agent is an antibody that recognizes an epitope or binding site on the surface of the cells or nuclei. In some implementations, the method may use a single or collection of polyclonal antibodies that recognize an epitope or binding site on the surface of the cells or nuclei. In some implementations, the method may use a single or collection of monoclonal antibodies that recognize an epitope or binding site on the surface of the cells or nuclei. In some implementations, the method may use a mixture of polyclonal and monoclonal antibodies that recognize an epitope or binding site on the surface of the cells or nuclei. In some implementations, the preferred agent is a liposome that merges or fuses indiscriminately with all of the cells or nuclei. In some implementations, the agent is a protein or mixture of proteins that binds to proteins, sugars or other entities on the surface of the cells or nuclei. In some implementations, the agent is a peptide or mixture of peptides that binds to proteins, sugars or other entities on the surface of the cells or nuclei. In some implementations, the polynucleotide concatenate contains a biotin at its 5′ end, and binds to the agent through strepavidin molecules linked to the agent. In some implementations, the polynucleotide represents a concatenate of multiple units separated at their junction by a restriction endonuclease site. In some implementations, the repeating units are different, targeting different cDNA or genomic DNA sequences. In some implementations, the concatenate is synthesized synthetically as a single polynucleotide as opposed to created through ligation. In some implementations, the first unit of the polynucleotide concatenate is a dummy unit that remains bound to the agent after restriction endonuclease digestion.

In some implementations of the above-described method, the method involves incorporating additional universal or common sequences at the 5′ and 3′ ends of the unit(s), enabling subsequent common amplification in separate steps after gene amplification. In some implementations, the method involves libraries of a plurality of agent bound polynucleotides. In some implementations, the method involves libraries of a plurality of agent bound polynucleotides that are converted to double-stranded DNA. In some implementations, what is further claimed is a process of co-localizing an agent bound polynucleotide containing a single element or a concatenate of elements, with beads coupled to other primers, and bulk primers, and single cells within a micro or nanometer sized space, such as a micro or nanodroplet, well, or vesicle (“microreactor” or “nanodroplet”), such that the nucleotide element or elements that results from DNA amplification contain both a cell barcode nucleotide tag and a sample barcode nucleotide tag. In some implementations, what is further claimed is a process of digesting the polynucleotide, inside of a micro or nanometer sized space, bound to the agent or released from it, using a restriction endonuclease capable of binding to the restriction endonuclease binding sites present at each of the junctions between repeating units in the concatemer of 1. In some implementations, what is further claimed is a process of utilizing an organized distribution of barcoded primers linked to the agent for tracking the sample of origin for amplification products generated inside of emulsions, microvessels, microreactors etc to the sample of origin. In some implementations, what is further claimed is a process of utilizing a cipher to create barcodes linking amplification products generated inside of emulsions, microvessels, microreactors etc to the sample of origin. In some implementations, what is further claimed is a process of isolating such polynucleotide-linked, agent-bound cells in the preceding into micro or nanodroplets comprised of lipids or agarose.

In even another illustrative example, what is claimed is a process of isolating such polynucleotide-linked, agent-bound cells into micro or nanodroplets comprised of droplets or wells, etched or not, into a solid 2-dimensional surface or substrate. In some implementations, what is further claimed is a process of co-localizing polynucleotide-linked, agent-bound cells into micro or nanometer sized spaces with single cells, captured through photon-assisted or electrokinetic gating. In some implementations, what is further claimed is a process where the primers constituted by the elements of the polynucleotides target specific human identity loci, such that each element contains a universal primer binding site, a discrete barcode and a set of gene specific primers or nucleotide class specific primer(s). In some implementations, what is further claimed is a process allowing a multiplexing of samples prior to integration of cells into the vessels, thereby reducing per sample costs for each SCNGS run. In some implementations, the method may be applied in the Forensic Sciences, or in routine diagnostics, or in Research and Development (R&D), or in Drug Discovery or Development.

In yet even another illustrative example, a three-step SCNGS process of the present disclosure is provided, which involves a microfluidic device enabling the generation of emulsions of solution within an immiscible carrier fluid, the creation of such emulsions containing single cells with reagents and enzymes needed for carrying out a set of molecular biology reactions, transition of the cells into an incubation chamber within which molecular biology reactions take place, the subsequent merging of the emulsions with a second set of reagents and/or enzymes, transition to a second incubation chamber within which a second set of molecular biology reaction(s) takes place, the subsequent merging of the emulsions with a third set of reagents and/or enzymes, creating emulsions suitable for polymerase chain reaction in preparation for sequencing.

In some implementations of the above-described process, the process is for enabling multiplexed SCNGS approach through the tagging of cells within a collection of samples. In some implementations, the process is used with a relatively simple set of antibody bound or bead bound barcode tags or types, and/or bead libraries, that enable the tagging of cells within microreactors or vessels such that only a fraction of the cells of the sample are tagged, constituting a “skimming” of the cellular diversity from the sample. In some implementations, the solution is aqueous, or is non-aqueous. In some implementations, the first set of molecular biology reactions is restriction endonuclease digestion and/or linear amplification using a DNA polymerase. In some implementations, the second set of molecular biology reactions is proteinase digestion. In some implementations, the third set of molecular biology reactions enables geometric DNA amplification using a DNA polymerase.

In another illustrative example, what is claimed is the use of a plate or array of barcoded primers with an agent that recognizes and binds to cells of a sample differentially for the purposes of negatively ‘skimming’ a sample of cells for multiplexed SCNGS via negative selection, in that the antibody epitope is expressed on cells of known identity in the sample, the antibody is linked to a chemical entity that binds a solid substrate, enabling these cells to be removed from the sample, leaving only those that do not express the epitope for multiplexed SCNGS analysis, thereby skimming the sample and obviating the need for positive ‘skimming’ using an agent-bound polynucleotide concatenate.

In some implementations of the use of the plate or array, the agent is an antibody recognizing a differentially-expressed epitope. In some implementations, solid substrate is a magnetic bead. In some implementations, the chemical entity is biotin. In some implementations, pre-prepared plates or other substrates containing wells/chambers/vessels are utilized, to segregate samples defined by oligonucleotides that contain the same sequences in each well except a unique identifier or barcode which is different in each well/chamber/vessel.

In yet another illustrative example of the present disclosure, what is claimed is the use of an array, grid or matrix of barcoded primers in 2D or 3D space, to enable tracking of sample identity among cells of a sample of cells that is not depleted of any cells, for the purposes of multiplexing samples for SCNGS. In some implementations, pre-prepared plates or other substrates containing wells/chambers/vessels are utilized, to segregate samples defined by oligonucleotides that contain the same sequences in each well except a unique identifier or barcode which is different in each well/chamber/vessel.

In yet a further illustrative example of the present disclosure, what may be provided is a computer-readable non-transitory storage medium storing instructions which, when executed by one or more computing devices, cause the one or more computing devices to perform any method described herein. In some implementations, a computer system may be configured to perform the techniques and/or methods for the analysis or process according to the embodiments described herein. A system of the present disclosure may include or be associated with multiple subsystems such as, for example, one or more machines, one or more computer systems, and one or more data repositories. In some implementations, the various subsystems of the system may be communicatively connected over one or more networks, which may include packet-switching or other types of network infrastructure devices (e.g., routers, switches, etc.) that are configured to facilitate information exchange between remote systems. In one embodiment, the system may be a device in which the various subsystems (e.g., such as machine(s), computer system(s), and possibly a data repository) are components that are communicatively and/or operatively coupled and integrated within the device. In some of these operational contexts, the data repository and/or computer system(s) of the embodiments may be configured within a cloud computing environment. In a cloud computing environment, the storage devices comprising a data repository and/or the computing devices comprising a computer system may be allocated and instantiated for use as a utility and on-demand; thus, the cloud computing environment provides as services the infrastructure (e.g., physical and virtual machines, raw/block storage, firewalls, load-balancers, aggregators, networks, storage clusters, etc.), the platforms (e.g., a computing device and/or a solution stack that may include an operating system, a programming language execution environment, a database server, a web server, an application server, etc.), and the software (e.g., applications, application programming interfaces or Application Programming Interfaces or APIs, etc.) necessary to perform any storage-related and/or computing tasks. Here, it is noted that in various embodiments, the techniques described herein can be performed by various systems and devices that include some or all of the above subsystems and components (e.g., such as sequencing machines, computer systems, and data repositories) in various configurations and form factors; thus, the example embodiments and configurations as described are to be regarded in an illustrative rather than a restrictive sense.

List of Polynucleotides

Polynucleotide 0
5 primer biotinylation-
ACACCGTGCGATAACACTCGATCAATCTTCCCGTCTCACCGGTCTCCCTGGGTGACAGAGCAAGACCCTGTACACCG
TGCGATAACACTCGATCAATCTTCCCGTCTCACCGGTCTCCCGATCCAAGTTGACTTGGCTGAGATGTACACCGTGC
GATAACACTCGATCAATCTTCCCGTCTCACCGGTCTCCCGCGTTTGTGTGTGCATCTGTAAGCATGTACACCGTGCG
ATAACACTCGATCAATCTTCCCGTCTCACCGGTCTCCCGTGCACACTTGGACAGCATTTCCTGTACACCGTGCGATA
ACACTCGATCAATCTTCCCGTCTCACCGGTCTCCCGAGGAAGGGCTGTGTTTCAGGGCTGTACACCGTGCGATAACA
CTCGATCAATCTTCCCGTCTCACCGGTCTCCCCGCTTTTCTGGCCAGAAACCTCTGTACACCGTGCGATAACACTCG
ATCAATCTTCCCGTCTCACCGGTCTCCCCCAGCTTCCCTGATTCTTCAGCTTGTACACCGTGCGATAACACTCGATC
AATCTTCCCGTCTCACCGGTCTCCCAAAATTAACTTCTCTGGTGTGTGGAGATGTACACCGTGCGATAACACTCGAT
CAATCTTCCCGTCTCACCGGTCTCCCCCTAACCTATCATCCATCCTTATCTCTTGTACACCGTGCGATAACACTCGA
TCAATCTTCCCGTCTCACCGGTCTCCCTGCACCCAACATTCTAACAAAAGGCTGTACACCGTGCGATAACACTCGAT
CAATCTTCCCGTCTCACCGGTCTCCCCAATAGGTTTTTAAGGAACAGGTGGTGTACACCGTGCGATAACACTCGATC
AATCTTCCCGTCTCACCGGTCTCCCGGGTGATTCCCATTGGCCTGTACACCGTGCGATAACACTCGATCAATCTTCC
CGTCTCACCGGTCTCCCGAAGTAGCTGCTGAGTGATTTGTCTGTACACCGTGCGATAACACTCGATCAATCTTCCCG
TCTCACCGGTCTCCCCCTCTCCACCCTATAGACCCTGTACACCGTGCGATAACACTCGATCAATCTTCCCGTCTCAC
CGGTCTCCCCTAATTAAAGTGGTGTCCCAGATAATCTGTACACCGTGCGATAACACTCGATCAATCTTCCCGTCTCA
CCGGTCTCCCCTCTGACCCATCTAACGCCTATCTGTACACCGTGCGATAACACTCGATCAATCTTCCCGTCTCACCG
GTCTCCCTGAGTAGCTGGGACTACAGGCATGTACACCGTGCGATAACACTCGATCAATCTTCCCGTCTCACCGGTCT
CCCTGACGCGGTCTCCGCGGTGTACACCGTGCGATAACACTCGATCAATCTTCCCGTCTCACCGGTCTCCCCTTTTT
ATTAAATGCTTTCCATGTATCAAGTTCTGTACACCGTGCGATAACACTCGATCAATCTTCCCGTCTCACCGGTCTCC
CCTTTCCCAATTCTCCTTCAGTCCTGTACACCGTGCGATAACACTCGATCAATCTTCCCGTCTCACCGGTCTCCCGA
TATCAGGGAAGATGAAAAAAGAGACTGTACACCGTGCGATAACACTCGATCAATCTTCCCGTCTCACCGGTCTCCCG
TCCCAGAGGCCCTTGTCAGTGTACACCGTGCGATAACACTCGATCAATCTTCCCGTCTCACCGGTCTCCCCCTGGGC
TCTGTAAAGAATAGTGTACACGAACCGATTGGTGCAGTCTTC
Number of TGTACAs found: 23
No Internal TGTACAs Exist-just Adaptors
Polynucleotide 1
5 primer biotinylation-
ACACCGTGCGATAACACTCGATCAATCTGAGGGATATGCATCACCTTTTGGGTGACAGAGCAAGACCCTGTACACCG
TGCGATAACACTCGATCAATCTGAGGGATATGCATCACCTTTGATCCAAGTTGACTTGGCTGAGATGTACACCGTGC
GATAACACTCGATCAATCTGAGGGATATGCATCACCTTTGCGTTTGTGTGTGCATCTGTAAGCATGTACACCGTGCG
ATAACACTCGATCAATCTGAGGGATATGCATCACCTTTGTGCACACTTGGACAGCATTTCCTGTACACCGTGCGATA
ACACTCGATCAATCTGAGGGATATGCATCACCTTTGAGGAAGGGCTGTGTTTCAGGGCTGTACACCGTGCGATAACA
CTCGATCAATCTGAGGGATATGCATCACCTTTCGCTTTTCTGGCCAGAAACCTCTGTACACCGTGCGATAACACTCG
ATCAATCTGAGGGATATGCATCACCTTTCCAGCTTCCCTGATTCTTCAGCTTGTACACCGTGCGATAACACTCGATC
AATCTGAGGGATATGCATCACCTTTAAAATTAACTTCTCTGGTGTGTGGAGATGTACACCGTGCGATAACACTCGAT
CAATCTGAGGGATATGCATCACCTTTCCTAACCTATCATCCATCCTTATCTCTTGTACACCGTGCGATAACACTCGA
TCAATCTGAGGGATATGCATCACCTTTTGCACCCAACATTCTAACAAAAGGCTGTACACCGTGCGATAACACTCGAT
CAATCTGAGGGATATGCATCACCTTTCAATAGGTTTTTAAGGAACAGGTGGTGTACACCGTGCGATAACACTCGATC
AATCTGAGGGATATGCATCACCTTTGGGTGATTCCCATTGGCCTGTACACCGTGCGATAACACTCGATCAATCTGAG
GGATATGCATCACCTTTGAAGTAGCTGCTGAGTGATTTGTCTGTACACCGTGCGATAACACTCGATCAATCTGAGGG
ATATGCATCACCTTTCCTCTCCACCCTATAGACCCTGTACACCGTGCGATAACACTCGATCAATCTGAGGGATATGC
ATCACCTTTCTAATTAAAGTGGTGTCCCAGATAATCTGTACACCGTGCGATAACACTCGATCAATCTGAGGGATATG
CATCACCTTTCTCTGACCCATCTAACGCCTATCTGTACACCGTGCGATAACACTCGATCAATCTGAGGGATATGCAT
CACCTTTTGAGTAGCTGGGACTACAGGCATGTACACCGTGCGATAACACTCGATCAATCTGAGGGATATGCATCACC
TTTTGACGCGGTCTCCGCGGTGTACACCGTGCGATAACACTCGATCAATCTGAGGGATATGCATCACCTTTCTTTTT
ATTAAATGCTTTCCATGTATCAAGTTCTGTACACCGTGCGATAACACTCGATCAATCTGAGGGATATGCATCACCTT
TCTTTCCCAATTCTCCTTCAGTCCTGTACACCGTGCGATAACACTCGATCAATCTGAGGGATATGCATCACCTTTGA
TATCAGGGAAGATGAAAAAAGAGACTGTACACCGTGCGATAACACTCGATCAATCTGAGGGATATGCATCACCTTTG
TCCCAGAGGCCCTTGTCAGTGTACACCGTGCGATAACACTCGATCAATCTGAGGGATATGCATCACCTTTCCTGGGC
TCTGTAAAGAATAGTGTACACGAACCGATTGGTGCAGTCTTC
Number of TGTACAs found: 23
No Internal TGTACAs Exist-just Adaptors
Polynucleotide 2
5 primer biotinylation-
ACACCGTGCGATAACACTCGATCAATCTAGCACACGTAATCGTTTCCGTGGGTGACAGAGCAAGACCCTGTACACCG
TGCGATAACACTCGATCAATCTAGCACACGTAATCGTTTCCGGATCCAAGTTGACTTGGCTGAGATGTACACCGTGC
GATAACACTCGATCAATCTAGCACACGTAATCGTTTCCGGCGTTTGTGTGTGCATCTGTAAGCATGTACACCGTGCG
ATAACACTCGATCAATCTAGCACACGTAATCGTTTCCGGTGCACACTTGGACAGCATTTCCTGTACACCGTGCGATA
ACACTCGATCAATCTAGCACACGTAATCGTTTCCGGAGGAAGGGCTGTGTTTCAGGGCTGTACACCGTGCGATAACA
CTCGATCAATCTAGCACACGTAATCGTTTCCGCGCTTTTCTGGCCAGAAACCTCTGTACACCGTGCGATAACACTCG
ATCAATCTAGCACACGTAATCGTTTCCGCCAGCTTCCCTGATTCTTCAGCTTGTACACCGTGCGATAACACTCGATC
AATCTAGCACACGTAATCGTTTCCGAAAATTAACTTCTCTGGTGTGTGGAGATGTACACCGTGCGATAACACTCGAT
CAATCTAGCACACGTAATCGTTTCCGCCTAACCTATCATCCATCCTTATCTCTTGTACACCGTGCGATAACACTCGA
TCAATCTAGCACACGTAATCGTTTCCGTGCACCCAACATTCTAACAAAAGGCTGTACACCGTGCGATAACACTCGAT
CAATCTAGCACACGTAATCGTTTCCGCAATAGGTTTTTAAGGAACAGGTGGTGTACACCGTGCGATAACACTCGATC
AATCTAGCACACGTAATCGTTTCCGGGGTGATTCCCATTGGCCTGTACACCGTGCGATAACACTCGATCAATCTAGC
ACACGTAATCGTTTCCGGAAGTAGCTGCTGAGTGATTTGTCTGTACACCGTGCGATAACACTCGATCAATCTAGCAC
ACGTAATCGTTTCCGCCTCTCCACCCTATAGACCCTGTACACCGTGCGATAACACTCGATCAATCTAGCACACGTAA
TCGTTTCCGCTAATTAAAGTGGTGTCCCAGATAATCTGTACACCGTGCGATAACACTCGATCAATCTAGCACACGTA
ATCGTTTCCGCTCTGACCCATCTAACGCCTATCTGTACACCGTGCGATAACACTCGATCAATCTAGCACACGTAATC
GTTTCCGTGAGTAGCTGGGACTACAGGCATGTACACCGTGCGATAACACTCGATCAATCTAGCACACGTAATCGTTT
CCGTGACGCGGTCTCCGCGGTGTACACCGTGCGATAACACTCGATCAATCTAGCACACGTAATCGTTTCCGCTTTTT
ATTAAATGCTTTCCATGTATCAAGTTCTGTACACCGTGCGATAACACTCGATCAATCTAGCACACGTAATCGTTTCC
GCTTTCCCAATTCTCCTTCAGTCCTGTACACCGTGCGATAACACTCGATCAATCTAGCACACGTAATCGTTTCCGGA
TATCAGGGAAGATGAAAAAAGAGACTGTACACCGTGCGATAACACTCGATCAATCTAGCACACGTAATCGTTTCCGG
TCCCAGAGGCCCTTGTCAGTGTACACCGTGCGATAACACTCGATCAATCTAGCACACGTAATCGTTTCCGCCTGGGC
TCTGTAAAGAATAGTGTACACGAACCGATTGGTGCAGTCTTC
Number of TGTACAs found: 23
No Internal TGTACAs Exist-just Adaptors
Polynucleotide 3
5 primer biotinylation-
ACACCGTGCGATAACACTCGATCAATCTATGTGGGTGAGGTCCGACCCTGGGTGACAGAGCAAGACCCTGTACACCG
TGCGATAACACTCGATCAATCTATGTGGGTGAGGTCCGACCCGATCCAAGTTGACTTGGCTGAGATGTACACCGTGC
GATAACACTCGATCAATCTATGTGGGTGAGGTCCGACCCGCGTTTGTGTGTGCATCTGTAAGCATGTACACCGTGCG
ATAACACTCGATCAATCTATGTGGGTGAGGTCCGACCCGTGCACACTTGGACAGCATTTCCTGTACACCGTGCGATA
ACACTCGATCAATCTATGTGGGTGAGGTCCGACCCGAGGAAGGGCTGTGTTTCAGGGCTGTACACCGTGCGATAACA
CTCGATCAATCTATGTGGGTGAGGTCCGACCCCGCTTTTCTGGCCAGAAACCTCTGTACACCGTGCGATAACACTCG
ATCAATCTATGTGGGTGAGGTCCGACCCCCAGCTTCCCTGATTCTTCAGCTTGTACACCGTGCGATAACACTCGATC
AATCTATGTGGGTGAGGTCCGACCCAAAATTAACTTCTCTGGTGTGTGGAGATGTACACCGTGCGATAACACTCGAT
CAATCTATGTGGGTGAGGTCCGACCCCCTAACCTATCATCCATCCTTATCTCTTGTACACCGTGCGATAACACTCGA
TCAATCTATGTGGGTGAGGTCCGACCCTGCACCCAACATTCTAACAAAAGGCTGTACACCGTGCGATAACACTCGAT
CAATCTATGTGGGTGAGGTCCGACCCCAATAGGTTTTTAAGGAACAGGTGGTGTACACCGTGCGATAACACTCGATC
AATCTATGTGGGTGAGGTCCGACCCGGGTGATTCCCATTGGCCTGTACACCGTGCGATAACACTCGATCAATCTATG
TGGGTGAGGTCCGACCCGAAGTAGCTGCTGAGTGATTTGTCTGTACACCGTGCGATAACACTCGATCAATCTATGTG
GGTGAGGTCCGACCCCCTCTCCACCCTATAGACCCTGTACACCGTGCGATAACACTCGATCAATCTATGTGGGTGAG
GTCCGACCCCTAATTAAAGTGGTGTCCCAGATAATCTGTACACCGTGCGATAACACTCGATCAATCTATGTGGGTGA
GGTCCGACCCCTCTGACCCATCTAACGCCTATCTGTACACCGTGCGATAACACTCGATCAATCTATGTGGGTGAGGT
CCGACCCTGAGTAGCTGGGACTACAGGCATGTACACCGTGCGATAACACTCGATCAATCTATGTGGGTGAGGTCCGA
CCCTGACGCGGTCTCCGCGGTGTACACCGTGCGATAACACTCGATCAATCTATGTGGGTGAGGTCCGACCCCTTTTT
ATTAAATGCTTTCCATGTATCAAGTTCTGTACACCGTGCGATAACACTCGATCAATCTATGTGGGTGAGGTCCGACC
CCTTTCCCAATTCTCCTTCAGTCCTGTACACCGTGCGATAACACTCGATCAATCTATGTGGGTGAGGTCCGACCCGA
TATCAGGGAAGATGAAAAAAGAGACTGTACACCGTGCGATAACACTCGATCAATCTATGTGGGTGAGGTCCGACCCG
TCCCAGAGGCCCTTGTCAGTGTACACCGTGCGATAACACTCGATCAATCTATGTGGGTGAGGTCCGACCCCCTGGGC
TCTGTAAAGAATAGTGTACACGAACCGATTGGTGCAGTCTTC
Number of TGTACAs found: 23
No Internal TGTACAs Exist-just Adaptors
Polynucleotide 4
5 primer biotinylation-
ACACCGTGCGATAACACTCGATCAATCTAATATTCCATGTTATTCGTCTGGGTGACAGAGCAAGACCCTGTACACCG
TGCGATAACACTCGATCAATCTAATATTCCATGTTATTCGTCGATCCAAGTTGACTTGGCTGAGATGTACACCGTGC
GATAACACTCGATCAATCTAATATTCCATGTTATTCGTCGCGTTTGTGTGTGCATCTGTAAGCATGTACACCGTGCG
ATAACACTCGATCAATCTAATATTCCATGTTATTCGTCGTGCACACTTGGACAGCATTTCCTGTACACCGTGCGATA
ACACTCGATCAATCTAATATTCCATGTTATTCGTCGAGGAAGGGCTGTGTTTCAGGGCTGTACACCGTGCGATAACA
CTCGATCAATCTAATATTCCATGTTATTCGTCCGCTTTTCTGGCCAGAAACCTCTGTACACCGTGCGATAACACTCG
ATCAATCTAATATTCCATGTTATTCGTCCCAGCTTCCCTGATTCTTCAGCTTGTACACCGTGCGATAACACTCGATC
AATCTAATATTCCATGTTATTCGTCAAAATTAACTTCTCTGGTGTGTGGAGATGTACACCGTGCGATAACACTCGAT
CAATCTAATATTCCATGTTATTCGTCCCTAACCTATCATCCATCCTTATCTCTTGTACACCGTGCGATAACACTCGA
TCAATCTAATATTCCATGTTATTCGTCTGCACCCAACATTCTAACAAAAGGCTGTACACCGTGCGATAACACTCGAT
CAATCTAATATTCCATGTTATTCGTCCAATAGGTTTTTAAGGAACAGGTGGTGTACACCGTGCGATAACACTCGATC
AATCTAATATTCCATGTTATTCGTCGGGTGATTCCCATTGGCCTGTACACCGTGCGATAACACTCGATCAATCTAAT
ATTCCATGTTATTCGTCGAAGTAGCTGCTGAGTGATTTGTCTGTACACCGTGCGATAACACTCGATCAATCTAATAT
TCCATGTTATTCGTCCCTCTCCACCCTATAGACCCTGTACACCGTGCGATAACACTCGATCAATCTAATATTCCATG
TTATTCGTCCTAATTAAAGTGGTGTCCCAGATAATCTGTACACCGTGCGATAACACTCGATCAATCTAATATTCCAT
GTTATTCGTCCTCTGACCCATCTAACGCCTATCTGTACACCGTGCGATAACACTCGATCAATCTAATATTCCATGTT
ATTCGTCTGAGTAGCTGGGACTACAGGCATGTACACCGTGCGATAACACTCGATCAATCTAATATTCCATGTTATTC
GTCTGACGCGGTCTCCGCGGTGTACACCGTGCGATAACACTCGATCAATCTAATATTCCATGTTATTCGTCCTTTTT
ATTAAATGCTTTCCATGTATCAAGTTCTGTACACCGTGCGATAACACTCGATCAATCTAATATTCCATGTTATTCGT
CCTTTCCCAATTCTCCTTCAGTCCTGTACACCGTGCGATAACACTCGATCAATCTAATATTCCATGTTATTCGTCGA
TATCAGGGAAGATGAAAAAAGAGACTGTACACCGTGCGATAACACTCGATCAATCTAATATTCCATGTTATTCGTCG
TCCCAGAGGCCCTTGTCAGTGTACACCGTGCGATAACACTCGATCAATCTAATATTCCATGTTATTCGTCCCTGGGC
TCTGTAAAGAATAGTGTACACGAACCGATTGGTGCAGTCTTC
Number of TGTACAs found: 23
No Internal TGTACAs Exist-just Adaptors
Polynucleotide 5
5 primer biotinylation-
ACACCGTGCGATAACACTCGATCAATCTTTTGTAGAAATACGGGGCCTTGGGTGACAGAGCAAGACCCTGTACACCG
TGCGATAACACTCGATCAATCTTTTGTAGAAATACGGGGCCTGATCCAAGTTGACTTGGCTGAGATGTACACCGTGC
GATAACACTCGATCAATCTTTTGTAGAAATACGGGGCCTGCGTTTGTGTGTGCATCTGTAAGCATGTACACCGTGCG
ATAACACTCGATCAATCTTTTGTAGAAATACGGGGCCTGTGCACACTTGGACAGCATTTCCTGTACACCGTGCGATA
ACACTCGATCAATCTTTTGTAGAAATACGGGGCCTGAGGAAGGGCTGTGTTTCAGGGCTGTACACCGTGCGATAACA
CTCGATCAATCTTTTGTAGAAATACGGGGCCTCGCTTTTCTGGCCAGAAACCTCTGTACACCGTGCGATAACACTCG
ATCAATCTTTTGTAGAAATACGGGGCCTCCAGCTTCCCTGATTCTTCAGCTTGTACACCGTGCGATAACACTCGATC
AATCTTTTGTAGAAATACGGGGCCTAAAATTAACTTCTCTGGTGTGTGGAGATGTACACCGTGCGATAACACTCGAT
CAATCTTTTGTAGAAATACGGGGCCTCCTAACCTATCATCCATCCTTATCTCTTGTACACCGTGCGATAACACTCGA
TCAATCTTTTGTAGAAATACGGGGCCTTGCACCCAACATTCTAACAAAAGGCTGTACACCGTGCGATAACACTCGAT
CAATCTTTTGTAGAAATACGGGGCCTCAATAGGTTTTTAAGGAACAGGTGGTGTACACCGTGCGATAACACTCGATC
AATCTTTTGTAGAAATACGGGGCCTGGGTGATTCCCATTGGCCTGTACACCGTGCGATAACACTCGATCAATCTTTT
GTAGAAATACGGGGCCTGAAGTAGCTGCTGAGTGATTTGTCTGTACACCGTGCGATAACACTCGATCAATCTTTTGT
AGAAATACGGGGCCTCCTCTCCACCCTATAGACCCTGTACACCGTGCGATAACACTCGATCAATCTTTTGTAGAAAT
ACGGGGCCTCTAATTAAAGTGGTGTCCCAGATAATCTGTACACCGTGCGATAACACTCGATCAATCTTTTGTAGAAA
TACGGGGCCTCTCTGACCCATCTAACGCCTATCTGTACACCGTGCGATAACACTCGATCAATCTTTTGTAGAAATAC
GGGGCCTTGAGTAGCTGGGACTACAGGCATGTACACCGTGCGATAACACTCGATCAATCTTTTGTAGAAATACGGGG
CCTTGACGCGGTCTCCGCGGTGTACACCGTGCGATAACACTCGATCAATCTTTTGTAGAAATACGGGGCCTCTTTTT
ATTAAATGCTTTCCATGTATCAAGTTCTGTACACCGTGCGATAACACTCGATCAATCTTTTGTAGAAATACGGGGCC
TCTTTCCCAATTCTCCTTCAGTCCTGTACACCGTGCGATAACACTCGATCAATCTTTTGTAGAAATACGGGGCCTGA
TATCAGGGAAGATGAAAAAAGAGACTGTACACCGTGCGATAACACTCGATCAATCTTTTGTAGAAATACGGGGCCTG
TCCCAGAGGCCCTTGTCAGTGTACACCGTGCGATAACACTCGATCAATCTTTTGTAGAAATACGGGGCCTCCTGGGC
TCTGTAAAGAATAGTGTACACGAACCGATTGGTGCAGTCTTC
Number of TGTACAs found: 23
No Internal TGTACAs Exist-just Adaptors
Polynucleotide 6
5 primer biotinylation-
ACACCGTGCGATAACACTCGATCAATCTCGAGCACAACCCTAAGTTTATGGGTGACAGAGCAAGACCCTGTACACCG
TGCGATAACACTCGATCAATCTCGAGCACAACCCTAAGTTTAGATCCAAGTTGACTTGGCTGAGATGTACACCGTGC
GATAACACTCGATCAATCTCGAGCACAACCCTAAGTTTAGCGTTTGTGTGTGCATCTGTAAGCATGTACACCGTGCG
ATAACACTCGATCAATCTCGAGCACAACCCTAAGTTTAGTGCACACTTGGACAGCATTTCCTGTACACCGTGCGATA
ACACTCGATCAATCTCGAGCACAACCCTAAGTTTAGAGGAAGGGCTGTGTTTCAGGGCTGTACACCGTGCGATAACA
CTCGATCAATCTCGAGCACAACCCTAAGTTTACGCTTTTCTGGCCAGAAACCTCTGTACACCGTGCGATAACACTCG
ATCAATCTCGAGCACAACCCTAAGTTTACCAGCTTCCCTGATTCTTCAGCTTGTACACCGTGCGATAACACTCGATC
AATCTCGAGCACAACCCTAAGTTTAAAAATTAACTTCTCTGGTGTGTGGAGATGTACACCGTGCGATAACACTCGAT
CAATCTCGAGCACAACCCTAAGTTTACCTAACCTATCATCCATCCTTATCTCTTGTACACCGTGCGATAACACTCGA
TCAATCTCGAGCACAACCCTAAGTTTATGCACCCAACATTCTAACAAAAGGCTGTACACCGTGCGATAACACTCGAT
CAATCTCGAGCACAACCCTAAGTTTACAATAGGTTTTTAAGGAACAGGTGGTGTACACCGTGCGATAACACTCGATC
AATCTCGAGCACAACCCTAAGTTTAGGGTGATTCCCATTGGCCTGTACACCGTGCGATAACACTCGATCAATCTCGA
GCACAACCCTAAGTTTAGAAGTAGCTGCTGAGTGATTTGTCTGTACACCGTGCGATAACACTCGATCAATCTCGAGC
ACAACCCTAAGTTTACCTCTCCACCCTATAGACCCTGTACACCGTGCGATAACACTCGATCAATCTCGAGCACAACC
CTAAGTTTACTAATTAAAGTGGTGTCCCAGATAATCTGTACACCGTGCGATAACACTCGATCAATCTCGAGCACAAC
CCTAAGTTTACTCTGACCCATCTAACGCCTATCTGTACACCGTGCGATAACACTCGATCAATCTCGAGCACAACCCT
AAGTTTATGAGTAGCTGGGACTACAGGCATGTACACCGTGCGATAACACTCGATCAATCTCGAGCACAACCCTAAGT
TTATGACGCGGTCTCCGCGGTGTACACCGTGCGATAACACTCGATCAATCTCGAGCACAACCCTAAGTTTACTTTTT
ATTAAATGCTTTCCATGTATCAAGTTCTGTACACCGTGCGATAACACTCGATCAATCTCGAGCACAACCCTAAGTTT
ACTTTCCCAATTCTCCTTCAGTCCTGTACACCGTGCGATAACACTCGATCAATCTCGAGCACAACCCTAAGTTTAGA
TATCAGGGAAGATGAAAAAAGAGACTGTACACCGTGCGATAACACTCGATCAATCTCGAGCACAACCCTAAGTTTAG
TCCCAGAGGCCCTTGTCAGTGTACACCGTGCGATAACACTCGATCAATCTCGAGCACAACCCTAAGTTTACCTGGGC
TCTGTAAAGAATAGTGTACACGAACCGATTGGTGCAGTCTTC
Number of TGTACAs found: 23
No Internal TGTACAs Exist-just Adaptors
Polynucleotide 7
5 primer biotinylation-
ACACCGTGCGATAACACTCGATCAATCTACCGCCGGTTTATGGTGGTGTGGGTGACAGAGCAAGACCCTGTACACCG
TGCGATAACACTCGATCAATCTACCGCCGGTTTATGGTGGTGGATCCAAGTTGACTTGGCTGAGATGTACACCGTGC
GATAACACTCGATCAATCTACCGCCGGTTTATGGTGGTGGCGTTTGTGTGTGCATCTGTAAGCATGTACACCGTGCG
ATAACACTCGATCAATCTACCGCCGGTTTATGGTGGTGGTGCACACTTGGACAGCATTTCCTGTACACCGTGCGATA
ACACTCGATCAATCTACCGCCGGTTTATGGTGGTGGAGGAAGGGCTGTGTTTCAGGGCTGTACACCGTGCGATAACA
CTCGATCAATCTACCGCCGGTTTATGGTGGTGCGCTTTTCTGGCCAGAAACCTCTGTACACCGTGCGATAACACTCG
ATCAATCTACCGCCGGTTTATGGTGGTGCCAGCTTCCCTGATTCTTCAGCTTGTACACCGTGCGATAACACTCGATC
AATCTACCGCCGGTTTATGGTGGTGAAAATTAACTTCTCTGGTGTGTGGAGATGTACACCGTGCGATAACACTCGAT
CAATCTACCGCCGGTTTATGGTGGTGCCTAACCTATCATCCATCCTTATCTCTTGTACACCGTGCGATAACACTCGA
TCAATCTACCGCCGGTTTATGGTGGTGTGCACCCAACATTCTAACAAAAGGCTGTACACCGTGCGATAACACTCGAT
CAATCTACCGCCGGTTTATGGTGGTGCAATAGGTTTTTAAGGAACAGGTGGTGTACACCGTGCGATAACACTCGATC
AATCTACCGCCGGTTTATGGTGGTGGGGTGATTCCCATTGGCCTGTACACCGTGCGATAACACTCGATCAATCTACC
GCCGGTTTATGGTGGTGGAAGTAGCTGCTGAGTGATTTGTCTGTACACCGTGCGATAACACTCGATCAATCTACCGC
CGGTTTATGGTGGTGCCTCTCCACCCTATAGACCCTGTACACCGTGCGATAACACTCGATCAATCTACCGCCGGTTT
ATGGTGGTGCTAATTAAAGTGGTGTCCCAGATAATCTGTACACCGTGCGATAACACTCGATCAATCTACCGCCGGTT
TATGGTGGTGCTCTGACCCATCTAACGCCTATCTGTACACCGTGCGATAACACTCGATCAATCTACCGCCGGTTTAT
GGTGGTGTGAGTAGCTGGGACTACAGGCATGTACACCGTGCGATAACACTCGATCAATCTACCGCCGGTTTATGGTG
GTGTGACGCGGTCTCCGCGGTGTACACCGTGCGATAACACTCGATCAATCTACCGCCGGTTTATGGTGGTGCTTTTT
ATTAAATGCTTTCCATGTATCAAGTTCTGTACACCGTGCGATAACACTCGATCAATCTACCGCCGGTTTATGGTGGT
GCTTTCCCAATTCTCCTTCAGTCCTGTACACCGTGCGATAACACTCGATCAATCTACCGCCGGTTTATGGTGGTGGA
TATCAGGGAAGATGAAAAAAGAGACTGTACACCGTGCGATAACACTCGATCAATCTACCGCCGGTTTATGGTGGTGG
TCCCAGAGGCCCTTGTCAGTGTACACCGTGCGATAACACTCGATCAATCTACCGCCGGTTTATGGTGGTGCCTGGGC
TCTGTAAAGAATAGTGTACACGAACCGATTGGTGCAGTCTTC
Number of TGTACAs found: 23
No Internal TGTACAs Exist-just Adaptors
Polynucleotide 8
5 primer biotinylation-
ACACCGTGCGATAACACTCGATCAATCTGTCAGCTCCGAAGAGCAGTTTGGGTGACAGAGCAAGACCCTGTACACCG
TGCGATAACACTCGATCAATCTGTCAGCTCCGAAGAGCAGTTGATCCAAGTTGACTTGGCTGAGATGTACACCGTGC
GATAACACTCGATCAATCTGTCAGCTCCGAAGAGCAGTTGCGTTTGTGTGTGCATCTGTAAGCATGTACACCGTGCG
ATAACACTCGATCAATCTGTCAGCTCCGAAGAGCAGTTGTGCACACTTGGACAGCATTTCCTGTACACCGTGCGATA
ACACTCGATCAATCTGTCAGCTCCGAAGAGCAGTTGAGGAAGGGCTGTGTTTCAGGGCTGTACACCGTGCGATAACA
CTCGATCAATCTGTCAGCTCCGAAGAGCAGTTCGCTTTTCTGGCCAGAAACCTCTGTACACCGTGCGATAACACTCG
ATCAATCTGTCAGCTCCGAAGAGCAGTTCCAGCTTCCCTGATTCTTCAGCTTGTACACCGTGCGATAACACTCGATC
AATCTGTCAGCTCCGAAGAGCAGTTAAAATTAACTTCTCTGGTGTGTGGAGATGTACACCGTGCGATAACACTCGAT
CAATCTGTCAGCTCCGAAGAGCAGTTCCTAACCTATCATCCATCCTTATCTCTTGTACACCGTGCGATAACACTCGA
TCAATCTGTCAGCTCCGAAGAGCAGTTTGCACCCAACATTCTAACAAAAGGCTGTACACCGTGCGATAACACTCGAT
CAATCTGTCAGCTCCGAAGAGCAGTTCAATAGGTTTTTAAGGAACAGGTGGTGTACACCGTGCGATAACACTCGATC
AATCTGTCAGCTCCGAAGAGCAGTTGGGTGATTCCCATTGGCCTGTACACCGTGCGATAACACTCGATCAATCTGTC
AGCTCCGAAGAGCAGTTGAAGTAGCTGCTGAGTGATTTGTCTGTACACCGTGCGATAACACTCGATCAATCTGTCAG
CTCCGAAGAGCAGTTCCTCTCCACCCTATAGACCCTGTACACCGTGCGATAACACTCGATCAATCTGTCAGCTCCGA
AGAGCAGTTCTAATTAAAGTGGTGTCCCAGATAATCTGTACACCGTGCGATAACACTCGATCAATCTGTCAGCTCCG
AAGAGCAGTTCTCTGACCCATCTAACGCCTATCTGTACACCGTGCGATAACACTCGATCAATCTGTCAGCTCCGAAG
AGCAGTTTGAGTAGCTGGGACTACAGGCATGTACACCGTGCGATAACACTCGATCAATCTGTCAGCTCCGAAGAGCA
GTTTGACGCGGTCTCCGCGGTGTACACCGTGCGATAACACTCGATCAATCTGTCAGCTCCGAAGAGCAGTTCTTTTT
ATTAAATGCTTTCCATGTATCAAGTTCTGTACACCGTGCGATAACACTCGATCAATCTGTCAGCTCCGAAGAGCAGT
TCTTTCCCAATTCTCCTTCAGTCCTGTACACCGTGCGATAACACTCGATCAATCTGTCAGCTCCGAAGAGCAGTTGA
TATCAGGGAAGATGAAAAAAGAGACTGTACACCGTGCGATAACACTCGATCAATCTGTCAGCTCCGAAGAGCAGTTG
TCCCAGAGGCCCTTGTCAGTGTACACCGTGCGATAACACTCGATCAATCTGTCAGCTCCGAAGAGCAGTTCCTGGGC
TCTGTAAAGAATAGTGTACACGAACCGATTGGTGCAGTCTTC
Number of TGTACAs found: 23
No Internal TGTACAs Exist-just Adaptors
Polynucleotide 9
5 primer biotinylation-
ACACCGTGCGATAACACTCGATCAATCTTCGTATGCAGGCGTGGTCGATGGGTGACAGAGCAAGACCCTGTACACCG
TGCGATAACACTCGATCAATCTTCGTATGCAGGCGTGGTCGAGATCCAAGTTGACTTGGCTGAGATGTACACCGTGC
GATAACACTCGATCAATCTTCGTATGCAGGCGTGGTCGAGCGTTTGTGTGTGCATCTGTAAGCATGTACACCGTGCG
ATAACACTCGATCAATCTTCGTATGCAGGCGTGGTCGAGTGCACACTTGGACAGCATTTCCTGTACACCGTGCGATA
ACACTCGATCAATCTTCGTATGCAGGCGTGGTCGAGAGGAAGGGCTGTGTTTCAGGGCTGTACACCGTGCGATAACA
CTCGATCAATCTTCGTATGCAGGCGTGGTCGACGCTTTTCTGGCCAGAAACCTCTGTACACCGTGCGATAACACTCG
ATCAATCTTCGTATGCAGGCGTGGTCGACCAGCTTCCCTGATTCTTCAGCTTGTACACCGTGCGATAACACTCGATC
AATCTTCGTATGCAGGCGTGGTCGAAAAATTAACTTCTCTGGTGTGTGGAGATGTACACCGTGCGATAACACTCGAT
CAATCTTCGTATGCAGGCGTGGTCGACCTAACCTATCATCCATCCTTATCTCTTGTACACCGTGCGATAACACTCGA
TCAATCTTCGTATGCAGGCGTGGTCGATGCACCCAACATTCTAACAAAAGGCTGTACACCGTGCGATAACACTCGAT
CAATCTTCGTATGCAGGCGTGGTCGACAATAGGTTTTTAAGGAACAGGTGGTGTACACCGTGCGATAACACTCGATC
AATCTTCGTATGCAGGCGTGGTCGAGGGTGATTCCCATTGGCCTGTACACCGTGCGATAACACTCGATCAATCTTCG
TATGCAGGCGTGGTCGAGAAGTAGCTGCTGAGTGATTTGTCTGTACACCGTGCGATAACACTCGATCAATCTTCGTA
TGCAGGCGTGGTCGACCTCTCCACCCTATAGACCCTGTACACCGTGCGATAACACTCGATCAATCTTCGTATGCAGG
CGTGGTCGACTAATTAAAGTGGTGTCCCAGATAATCTGTACACCGTGCGATAACACTCGATCAATCTTCGTATGCAG
GCGTGGTCGACTCTGACCCATCTAACGCCTATCTGTACACCGTGCGATAACACTCGATCAATCTTCGTATGCAGGCG
TGGTCGATGAGTAGCTGGGACTACAGGCATGTACACCGTGCGATAACACTCGATCAATCTTCGTATGCAGGCGTGGT
CGATGACGCGGTCTCCGCGGTGTACACCGTGCGATAACACTCGATCAATCTTCGTATGCAGGCGTGGTCGACTTTTT
ATTAAATGCTTTCCATGTATCAAGTTCTGTACACCGTGCGATAACACTCGATCAATCTTCGTATGCAGGCGTGGTCG
ACTTTCCCAATTCTCCTTCAGTCCTGTACACCGTGCGATAACACTCGATCAATCTTCGTATGCAGGCGTGGTCGAGA
TATCAGGGAAGATGAAAAAAGAGACTGTACACCGTGCGATAACACTCGATCAATCTTCGTATGCAGGCGTGGTCGAG
TCCCAGAGGCCCTTGTCAGTGTACACCGTGCGATAACACTCGATCAATCTTCGTATGCAGGCGTGGTCGACCTGGGC
TCTGTAAAGAATAGTGTACACGAACCGATTGGTGCAGTCTTC
Number of TGTACAs found: 23
No Internal TGTACAs Exist-just Adaptors
Polynucleotide 10
5 primer biotinylation-
ACACCGTGCGATAACACTCGATCAATCTGCTCACGGATATGCGGTTCATGGGTGACAGAGCAAGACCCTGTACACCG
TGCGATAACACTCGATCAATCTGCTCACGGATATGCGGTTCAGATCCAAGTTGACTTGGCTGAGATGTACACCGTGC
GATAACACTCGATCAATCTGCTCACGGATATGCGGTTCAGCGTTTGTGTGTGCATCTGTAAGCATGTACACCGTGCG
ATAACACTCGATCAATCTGCTCACGGATATGCGGTTCAGTGCACACTTGGACAGCATTTCCTGTACACCGTGCGATA
ACACTCGATCAATCTGCTCACGGATATGCGGTTCAGAGGAAGGGCTGTGTTTCAGGGCTGTACACCGTGCGATAACA
CTCGATCAATCTGCTCACGGATATGCGGTTCACGCTTTTCTGGCCAGAAACCTCTGTACACCGTGCGATAACACTCG
ATCAATCTGCTCACGGATATGCGGTTCACCAGCTTCCCTGATTCTTCAGCTTGTACACCGTGCGATAACACTCGATC
AATCTGCTCACGGATATGCGGTTCAAAAATTAACTTCTCTGGTGTGTGGAGATGTACACCGTGCGATAACACTCGAT
CAATCTGCTCACGGATATGCGGTTCACCTAACCTATCATCCATCCTTATCTCTTGTACACCGTGCGATAACACTCGA
TCAATCTGCTCACGGATATGCGGTTCATGCACCCAACATTCTAACAAAAGGCTGTACACCGTGCGATAACACTCGAT
CAATCTGCTCACGGATATGCGGTTCACAATAGGTTTTTAAGGAACAGGTGGTGTACACCGTGCGATAACACTCGATC
AATCTGCTCACGGATATGCGGTTCAGGGTGATTCCCATTGGCCTGTACACCGTGCGATAACACTCGATCAATCTGCT
CACGGATATGCGGTTCAGAAGTAGCTGCTGAGTGATTTGTCTGTACACCGTGCGATAACACTCGATCAATCTGCTCA
CGGATATGCGGTTCACCTCTCCACCCTATAGACCCTGTACACCGTGCGATAACACTCGATCAATCTGCTCACGGATA
TGCGGTTCACTAATTAAAGTGGTGTCCCAGATAATCTGTACACCGTGCGATAACACTCGATCAATCTGCTCACGGAT
ATGCGGTTCACTCTGACCCATCTAACGCCTATCTGTACACCGTGCGATAACACTCGATCAATCTGCTCACGGATATG
CGGTTCATGAGTAGCTGGGACTACAGGCATGTACACCGTGCGATAACACTCGATCAATCTGCTCACGGATATGCGGT
TCATGACGCGGTCTCCGCGGTGTACACCGTGCGATAACACTCGATCAATCTGCTCACGGATATGCGGTTCACTTTTT
ATTAAATGCTTTCCATGTATCAAGTTCTGTACACCGTGCGATAACACTCGATCAATCTGCTCACGGATATGCGGTTC
ACTTTCCCAATTCTCCTTCAGTCCTGTACACCGTGCGATAACACTCGATCAATCTGCTCACGGATATGCGGTTCAGA
TATCAGGGAAGATGAAAAAAGAGACTGTACACCGTGCGATAACACTCGATCAATCTGCTCACGGATATGCGGTTCAG
TCCCAGAGGCCCTTGTCAGTGTACACCGTGCGATAACACTCGATCAATCTGCTCACGGATATGCGGTTCACCTGGGC
TCTGTAAAGAATAGTGTACACGAACCGATTGGTGCAGTCTTC
Number of TGTACAs found: 23
No Internal TGTACAs Exist-just Adaptors
Polynucleotide 11
5 primer biotinylation-
ACACCGTGCGATAACACTCGATCAATCTATGCGTGACGCTAAACTGCTTGGGTGACAGAGCAAGACCCTGTACACCG
TGCGATAACACTCGATCAATCTATGCGTGACGCTAAACTGCTGATCCAAGTTGACTTGGCTGAGATGTACACCGTGC
GATAACACTCGATCAATCTATGCGTGACGCTAAACTGCTGCGTTTGTGTGTGCATCTGTAAGCATGTACACCGTGCG
ATAACACTCGATCAATCTATGCGTGACGCTAAACTGCTGTGCACACTTGGACAGCATTTCCTGTACACCGTGCGATA
ACACTCGATCAATCTATGCGTGACGCTAAACTGCTGAGGAAGGGCTGTGTTTCAGGGCTGTACACCGTGCGATAACA
CTCGATCAATCTATGCGTGACGCTAAACTGCTCGCTTTTCTGGCCAGAAACCTCTGTACACCGTGCGATAACACTCG
ATCAATCTATGCGTGACGCTAAACTGCTCCAGCTTCCCTGATTCTTCAGCTTGTACACCGTGCGATAACACTCGATC
AATCTATGCGTGACGCTAAACTGCTAAAATTAACTTCTCTGGTGTGTGGAGATGTACACCGTGCGATAACACTCGAT
CAATCTATGCGTGACGCTAAACTGCTCCTAACCTATCATCCATCCTTATCTCTTGTACACCGTGCGATAACACTCGA
TCAATCTATGCGTGACGCTAAACTGCTTGCACCCAACATTCTAACAAAAGGCTGTACACCGTGCGATAACACTCGAT
CAATCTATGCGTGACGCTAAACTGCTCAATAGGTTTTTAAGGAACAGGTGGTGTACACCGTGCGATAACACTCGATC
AATCTATGCGTGACGCTAAACTGCTGGGTGATTCCCATTGGCCTGTACACCGTGCGATAACACTCGATCAATCTATG
CGTGACGCTAAACTGCTGAAGTAGCTGCTGAGTGATTTGTCTGTACACCGTGCGATAACACTCGATCAATCTATGCG
TGACGCTAAACTGCTCCTCTCCACCCTATAGACCCTGTACACCGTGCGATAACACTCGATCAATCTATGCGTGACGC
TAAACTGCTCTAATTAAAGTGGTGTCCCAGATAATCTGTACACCGTGCGATAACACTCGATCAATCTATGCGTGACG
CTAAACTGCTCTCTGACCCATCTAACGCCTATCTGTACACCGTGCGATAACACTCGATCAATCTATGCGTGACGCTA
AACTGCTTGAGTAGCTGGGACTACAGGCATGTACACCGTGCGATAACACTCGATCAATCTATGCGTGACGCTAAACT
GCTTGACGCGGTCTCCGCGGTGTACACCGTGCGATAACACTCGATCAATCTATGCGTGACGCTAAACTGCTCTTTTT
ATTAAATGCTTTCCATGTATCAAGTTCTGTACACCGTGCGATAACACTCGATCAATCTATGCGTGACGCTAAACTGC
TCTTTCCCAATTCTCCTTCAGTCCTGTACACCGTGCGATAACACTCGATCAATCTATGCGTGACGCTAAACTGCTGA
TATCAGGGAAGATGAAAAAAGAGACTGTACACCGTGCGATAACACTCGATCAATCTATGCGTGACGCTAAACTGCTG
TCCCAGAGGCCCTTGTCAGTGTACACCGTGCGATAACACTCGATCAATCTATGCGTGACGCTAAACTGCTCCTGGGC
TCTGTAAAGAATAGTGTACACGAACCGATTGGTGCAGTCTTC
Number of TGTACAs found: 23
No Internal TGTACAs Exist-just Adaptors
Polynucleotide 12
5 primer biotinylation-
ACACCGTGCGATAACACTCGATCAATCTGTGTCTTCGCGTTTACGGACTGGGTGACAGAGCAAGACCCTGTACACCG
TGCGATAACACTCGATCAATCTGTGTCTTCGCGTTTACGGACGATCCAAGTTGACTTGGCTGAGATGTACACCGTGC
GATAACACTCGATCAATCTGTGTCTTCGCGTTTACGGACGCGTTTGTGTGTGCATCTGTAAGCATGTACACCGTGCG
ATAACACTCGATCAATCTGTGTCTTCGCGTTTACGGACGTGCACACTTGGACAGCATTTCCTGTACACCGTGCGATA
ACACTCGATCAATCTGTGTCTTCGCGTTTACGGACGAGGAAGGGCTGTGTTTCAGGGCTGTACACCGTGCGATAACA
CTCGATCAATCTGTGTCTTCGCGTTTACGGACCGCTTTTCTGGCCAGAAACCTCTGTACACCGTGCGATAACACTCG
ATCAATCTGTGTCTTCGCGTTTACGGACCCAGCTTCCCTGATTCTTCAGCTTGTACACCGTGCGATAACACTCGATC
AATCTGTGTCTTCGCGTTTACGGACAAAATTAACTTCTCTGGTGTGTGGAGATGTACACCGTGCGATAACACTCGAT
CAATCTGTGTCTTCGCGTTTACGGACCCTAACCTATCATCCATCCTTATCTCTTGTACACCGTGCGATAACACTCGA
TCAATCTGTGTCTTCGCGTTTACGGACTGCACCCAACATTCTAACAAAAGGCTGTACACCGTGCGATAACACTCGAT
CAATCTGTGTCTTCGCGTTTACGGACCAATAGGTTTTTAAGGAACAGGTGGTGTACACCGTGCGATAACACTCGATC
AATCTGTGTCTTCGCGTTTACGGACGGGTGATTCCCATTGGCCTGTACACCGTGCGATAACACTCGATCAATCTGTG
TCTTCGCGTTTACGGACGAAGTAGCTGCTGAGTGATTTGTCTGTACACCGTGCGATAACACTCGATCAATCTGTGTC
TTCGCGTTTACGGACCCTCTCCACCCTATAGACCCTGTACACCGTGCGATAACACTCGATCAATCTGTGTCTTCGCG
TTTACGGACCTAATTAAAGTGGTGTCCCAGATAATCTGTACACCGTGCGATAACACTCGATCAATCTGTGTCTTCGC
GTTTACGGACCTCTGACCCATCTAACGCCTATCTGTACACCGTGCGATAACACTCGATCAATCTGTGTCTTCGCGTT
TACGGACTGAGTAGCTGGGACTACAGGCATGTACACCGTGCGATAACACTCGATCAATCTGTGTCTTCGCGTTTACG
GACTGACGCGGTCTCCGCGGTGTACACCGTGCGATAACACTCGATCAATCTGTGTCTTCGCGTTTACGGACCTTTTT
ATTAAATGCTTTCCATGTATCAAGTTCTGTACACCGTGCGATAACACTCGATCAATCTGTGTCTTCGCGTTTACGGA
CCTTTCCCAATTCTCCTTCAGTCCTGTACACCGTGCGATAACACTCGATCAATCTGTGTCTTCGCGTTTACGGACGA
TATCAGGGAAGATGAAAAAAGAGACTGTACACCGTGCGATAACACTCGATCAATCTGTGTCTTCGCGTTTACGGACG
TCCCAGAGGCCCTTGTCAGTGTACACCGTGCGATAACACTCGATCAATCTGTGTCTTCGCGTTTACGGACCCTGGGC
TCTGTAAAGAATAGTGTACACGAACCGATTGGTGCAGTCTTC
Number of TGTACAs found: 23
No Internal TGTACAs Exist-just Adaptors
Polynucleotide 13
5 primer biotinylation-
ACACCGTGCGATAACACTCGATCAATCTCTGATGCGACTAACATAATATGGGTGACAGAGCAAGACCCTGTACACCG
TGCGATAACACTCGATCAATCTCTGATGCGACTAACATAATAGATCCAAGTTGACTTGGCTGAGATGTACACCGTGC
GATAACACTCGATCAATCTCTGATGCGACTAACATAATAGCGTTTGTGTGTGCATCTGTAAGCATGTACACCGTGCG
ATAACACTCGATCAATCTCTGATGCGACTAACATAATAGTGCACACTTGGACAGCATTTCCTGTACACCGTGCGATA
ACACTCGATCAATCTCTGATGCGACTAACATAATAGAGGAAGGGCTGTGTTTCAGGGCTGTACACCGTGCGATAACA
CTCGATCAATCTCTGATGCGACTAACATAATACGCTTTTCTGGCCAGAAACCTCTGTACACCGTGCGATAACACTCG
ATCAATCTCTGATGCGACTAACATAATACCAGCTTCCCTGATTCTTCAGCTTGTACACCGTGCGATAACACTCGATC
AATCTCTGATGCGACTAACATAATAAAAATTAACTTCTCTGGTGTGTGGAGATGTACACCGTGCGATAACACTCGAT
CAATCTCTGATGCGACTAACATAATACCTAACCTATCATCCATCCTTATCTCTTGTACACCGTGCGATAACACTCGA
TCAATCTCTGATGCGACTAACATAATATGCACCCAACATTCTAACAAAAGGCTGTACACCGTGCGATAACACTCGAT
CAATCTCTGATGCGACTAACATAATACAATAGGTTTTTAAGGAACAGGTGGTGTACACCGTGCGATAACACTCGATC
AATCTCTGATGCGACTAACATAATAGGGTGATTCCCATTGGCCTGTACACCGTGCGATAACACTCGATCAATCTCTG
ATGCGACTAACATAATAGAAGTAGCTGCTGAGTGATTTGTCTGTACACCGTGCGATAACACTCGATCAATCTCTGAT
GCGACTAACATAATACCTCTCCACCCTATAGACCCTGTACACCGTGCGATAACACTCGATCAATCTCTGATGCGACT
AACATAATACTAATTAAAGTGGTGTCCCAGATAATCTGTACACCGTGCGATAACACTCGATCAATCTCTGATGCGAC
TAACATAATACTCTGACCCATCTAACGCCTATCTGTACACCGTGCGATAACACTCGATCAATCTCTGATGCGACTAA
CATAATATGAGTAGCTGGGACTACAGGCATGTACACCGTGCGATAACACTCGATCAATCTCTGATGCGACTAACATA
ATATGACGCGGTCTCCGCGGTGTACACCGTGCGATAACACTCGATCAATCTCTGATGCGACTAACATAATACTTTTT
ATTAAATGCTTTCCATGTATCAAGTTCTGTACACCGTGCGATAACACTCGATCAATCTCTGATGCGACTAACATAAT
ACTTTCCCAATTCTCCTTCAGTCCTGTACACCGTGCGATAACACTCGATCAATCTCTGATGCGACTAACATAATAGA
TATCAGGGAAGATGAAAAAAGAGACTGTACACCGTGCGATAACACTCGATCAATCTCTGATGCGACTAACATAATAG
TCCCAGAGGCCCTTGTCAGTGTACACCGTGCGATAACACTCGATCAATCTCTGATGCGACTAACATAATACCTGGGC
TCTGTAAAGAATAGTGTACACGAACCGATTGGTGCAGTCTTC
Number of TGTACAs found: 23
No Internal TGTACAs Exist-just Adaptors
Polynucleotide 14
5 primer biotinylation-
ACACCGTGCGATAACACTCGATCAATCTAACGACCCCGTCCATGGGTATGGGTGACAGAGCAAGACCCTGTACACCG
TGCGATAACACTCGATCAATCTAACGACCCCGTCCATGGGTAGATCCAAGTTGACTTGGCTGAGATGTACACCGTGC
GATAACACTCGATCAATCTAACGACCCCGTCCATGGGTAGCGTTTGTGTGTGCATCTGTAAGCATGTACACCGTGCG
ATAACACTCGATCAATCTAACGACCCCGTCCATGGGTAGTGCACACTTGGACAGCATTTCCTGTACACCGTGCGATA
ACACTCGATCAATCTAACGACCCCGTCCATGGGTAGAGGAAGGGCTGTGTTTCAGGGCTGTACACCGTGCGATAACA
CTCGATCAATCTAACGACCCCGTCCATGGGTACGCTTTTCTGGCCAGAAACCTCTGTACACCGTGCGATAACACTCG
ATCAATCTAACGACCCCGTCCATGGGTACCAGCTTCCCTGATTCTTCAGCTTGTACACCGTGCGATAACACTCGATC
AATCTAACGACCCCGTCCATGGGTAAAAATTAACTTCTCTGGTGTGTGGAGATGTACACCGTGCGATAACACTCGAT
CAATCTAACGACCCCGTCCATGGGTACCTAACCTATCATCCATCCTTATCTCTTGTACACCGTGCGATAACACTCGA
TCAATCTAACGACCCCGTCCATGGGTATGCACCCAACATTCTAACAAAAGGCTGTACACCGTGCGATAACACTCGAT
CAATCTAACGACCCCGTCCATGGGTACAATAGGTTTTTAAGGAACAGGTGGTGTACACCGTGCGATAACACTCGATC
AATCTAACGACCCCGTCCATGGGTAGGGTGATTCCCATTGGCCTGTACACCGTGCGATAACACTCGATCAATCTAAC
GACCCCGTCCATGGGTAGAAGTAGCTGCTGAGTGATTTGTCTGTACACCGTGCGATAACACTCGATCAATCTAACGA
CCCCGTCCATGGGTACCTCTCCACCCTATAGACCCTGTACACCGTGCGATAACACTCGATCAATCTAACGACCCCGT
CCATGGGTACTAATTAAAGTGGTGTCCCAGATAATCTGTACACCGTGCGATAACACTCGATCAATCTAACGACCCCG
TCCATGGGTACTCTGACCCATCTAACGCCTATCTGTACACCGTGCGATAACACTCGATCAATCTAACGACCCCGTCC
ATGGGTATGAGTAGCTGGGACTACAGGCATGTACACCGTGCGATAACACTCGATCAATCTAACGACCCCGTCCATGG
GTATGACGCGGTCTCCGCGGTGTACACCGTGCGATAACACTCGATCAATCTAACGACCCCGTCCATGGGTACTTTTT
ATTAAATGCTTTCCATGTATCAAGTTCTGTACACCGTGCGATAACACTCGATCAATCTAACGACCCCGTCCATGGGT
ACTTTCCCAATTCTCCTTCAGTCCTGTACACCGTGCGATAACACTCGATCAATCTAACGACCCCGTCCATGGGTAGA
TATCAGGGAAGATGAAAAAAGAGACTGTACACCGTGCGATAACACTCGATCAATCTAACGACCCCGTCCATGGGTAG
TCCCAGAGGCCCTTGTCAGTGTACACCGTGCGATAACACTCGATCAATCTAACGACCCCGTCCATGGGTACCTGGGC
TCTGTAAAGAATAGTGTACACGAACCGATTGGTGCAGTCTTC
Number of TGTACAs found: 23
No Internal TGTACAs Exist-just Adaptors
Polynucleotide 15
5 primer biotinylation-
ACACCGTGCGATAACACTCGATCAATCTTCTTTACGCGTTGATTCCTATGGGTGACAGAGCAAGACCCTGTACACCG
TGCGATAACACTCGATCAATCTTCTTTACGCGTTGATTCCTAGATCCAAGTTGACTTGGCTGAGATGTACACCGTGC
GATAACACTCGATCAATCTTCTTTACGCGTTGATTCCTAGCGTTTGTGTGTGCATCTGTAAGCATGTACACCGTGCG
ATAACACTCGATCAATCTTCTTTACGCGTTGATTCCTAGTGCACACTTGGACAGCATTTCCTGTACACCGTGCGATA
ACACTCGATCAATCTTCTTTACGCGTTGATTCCTAGAGGAAGGGCTGTGTTTCAGGGCTGTACACCGTGCGATAACA
CTCGATCAATCTTCTTTACGCGTTGATTCCTACGCTTTTCTGGCCAGAAACCTCTGTACACCGTGCGATAACACTCG
ATCAATCTTCTTTACGCGTTGATTCCTACCAGCTTCCCTGATTCTTCAGCTTGTACACCGTGCGATAACACTCGATC
AATCTTCTTTACGCGTTGATTCCTAAAAATTAACTTCTCTGGTGTGTGGAGATGTACACCGTGCGATAACACTCGAT
CAATCTTCTTTACGCGTTGATTCCTACCTAACCTATCATCCATCCTTATCTCTTGTACACCGTGCGATAACACTCGA
TCAATCTTCTTTACGCGTTGATTCCTATGCACCCAACATTCTAACAAAAGGCTGTACACCGTGCGATAACACTCGAT
CAATCTTCTTTACGCGTTGATTCCTACAATAGGTTTTTAAGGAACAGGTGGTGTACACCGTGCGATAACACTCGATC
AATCTTCTTTACGCGTTGATTCCTAGGGTGATTCCCATTGGCCTGTACACCGTGCGATAACACTCGATCAATCTTCT
TTACGCGTTGATTCCTAGAAGTAGCTGCTGAGTGATTTGTCTGTACACCGTGCGATAACACTCGATCAATCTTCTTT
ACGCGTTGATTCCTACCTCTCCACCCTATAGACCCTGTACACCGTGCGATAACACTCGATCAATCTTCTTTACGCGT
TGATTCCTACTAATTAAAGTGGTGTCCCAGATAATCTGTACACCGTGCGATAACACTCGATCAATCTTCTTTACGCG
TTGATTCCTACTCTGACCCATCTAACGCCTATCTGTACACCGTGCGATAACACTCGATCAATCTTCTTTACGCGTTG
ATTCCTATGAGTAGCTGGGACTACAGGCATGTACACCGTGCGATAACACTCGATCAATCTTCTTTACGCGTTGATTC
CTATGACGCGGTCTCCGCGGTGTACACCGTGCGATAACACTCGATCAATCTTCTTTACGCGTTGATTCCTACTTTTT
ATTAAATGCTTTCCATGTATCAAGTTCTGTACACCGTGCGATAACACTCGATCAATCTTCTTTACGCGTTGATTCCT
ACTTTCCCAATTCTCCTTCAGTCCTGTACACCGTGCGATAACACTCGATCAATCTTCTTTACGCGTTGATTCCTAGA
TATCAGGGAAGATGAAAAAAGAGACTGTACACCGTGCGATAACACTCGATCAATCTTCTTTACGCGTTGATTCCTAG
TCCCAGAGGCCCTTGTCAGTGTACACCGTGCGATAACACTCGATCAATCTTCTTTACGCGTTGATTCCTACCTGGGC
TCTGTAAAGAATAGTGTACACGAACCGATTGGTGCAGTCTTC
Number of TGTACAs found: 23
No Internal TGTACAs Exist-just Adaptors
Polynucleotide 16
5 primer biotinylation-
ACACCGTGCGATAACACTCGATCAATCTCCTAGTAGTGACCTCCCGTCTGGGTGACAGAGCAAGACCCTGTACACCG
TGCGATAACACTCGATCAATCTCCTAGTAGTGACCTCCCGTCGATCCAAGTTGACTTGGCTGAGATGTACACCGTGC
GATAACACTCGATCAATCTCCTAGTAGTGACCTCCCGTCGCGTTTGTGTGTGCATCTGTAAGCATGTACACCGTGCG
ATAACACTCGATCAATCTCCTAGTAGTGACCTCCCGTCGTGCACACTTGGACAGCATTTCCTGTACACCGTGCGATA
ACACTCGATCAATCTCCTAGTAGTGACCTCCCGTCGAGGAAGGGCTGTGTTTCAGGGCTGTACACCGTGCGATAACA
CTCGATCAATCTCCTAGTAGTGACCTCCCGTCCGCTTTTCTGGCCAGAAACCTCTGTACACCGTGCGATAACACTCG
ATCAATCTCCTAGTAGTGACCTCCCGTCCCAGCTTCCCTGATTCTTCAGCTTGTACACCGTGCGATAACACTCGATC
AATCTCCTAGTAGTGACCTCCCGTCAAAATTAACTTCTCTGGTGTGTGGAGATGTACACCGTGCGATAACACTCGAT
CAATCTCCTAGTAGTGACCTCCCGTCCCTAACCTATCATCCATCCTTATCTCTTGTACACCGTGCGATAACACTCGA
TCAATCTCCTAGTAGTGACCTCCCGTCTGCACCCAACATTCTAACAAAAGGCTGTACACCGTGCGATAACACTCGAT
CAATCTCCTAGTAGTGACCTCCCGTCCAATAGGTTTTTAAGGAACAGGTGGTGTACACCGTGCGATAACACTCGATC
AATCTCCTAGTAGTGACCTCCCGTCGGGTGATTCCCATTGGCCTGTACACCGTGCGATAACACTCGATCAATCTCCT
AGTAGTGACCTCCCGTCGAAGTAGCTGCTGAGTGATTTGTCTGTACACCGTGCGATAACACTCGATCAATCTCCTAG
TAGTGACCTCCCGTCCCTCTCCACCCTATAGACCCTGTACACCGTGCGATAACACTCGATCAATCTCCTAGTAGTGA
CCTCCCGTCCTAATTAAAGTGGTGTCCCAGATAATCTGTACACCGTGCGATAACACTCGATCAATCTCCTAGTAGTG
ACCTCCCGTCCTCTGACCCATCTAACGCCTATCTGTACACCGTGCGATAACACTCGATCAATCTCCTAGTAGTGACC
TCCCGTCTGAGTAGCTGGGACTACAGGCATGTACACCGTGCGATAACACTCGATCAATCTCCTAGTAGTGACCTCCC
GTCTGACGCGGTCTCCGCGGTGTACACCGTGCGATAACACTCGATCAATCTCCTAGTAGTGACCTCCCGTCCTTTTT
ATTAAATGCTTTCCATGTATCAAGTTCTGTACACCGTGCGATAACACTCGATCAATCTCCTAGTAGTGACCTCCCGT
CCTTTCCCAATTCTCCTTCAGTCCTGTACACCGTGCGATAACACTCGATCAATCTCCTAGTAGTGACCTCCCGTCGA
TATCAGGGAAGATGAAAAAAGAGACTGTACACCGTGCGATAACACTCGATCAATCTCCTAGTAGTGACCTCCCGTCG
TCCCAGAGGCCCTTGTCAGTGTACACCGTGCGATAACACTCGATCAATCTCCTAGTAGTGACCTCCCGTCCCTGGGC
TCTGTAAAGAATAGTGTACACGAACCGATTGGTGCAGTCTTC
Number of TGTACAs found: 23
No Internal TGTACAs Exist-just Adaptors
Polynucleotide 17
5 primer biotinylation-
ACACCGTGCGATAACACTCGATCAATCTATGCGGTATAATATGTTGGCTGGGTGACAGAGCAAGACCCTGTACACCG
TGCGATAACACTCGATCAATCTATGCGGTATAATATGTTGGCGATCCAAGTTGACTTGGCTGAGATGTACACCGTGC
GATAACACTCGATCAATCTATGCGGTATAATATGTTGGCGCGTTTGTGTGTGCATCTGTAAGCATGTACACCGTGCG
ATAACACTCGATCAATCTATGCGGTATAATATGTTGGCGTGCACACTTGGACAGCATTTCCTGTACACCGTGCGATA
ACACTCGATCAATCTATGCGGTATAATATGTTGGCGAGGAAGGGCTGTGTTTCAGGGCTGTACACCGTGCGATAACA
CTCGATCAATCTATGCGGTATAATATGTTGGCCGCTTTTCTGGCCAGAAACCTCTGTACACCGTGCGATAACACTCG
ATCAATCTATGCGGTATAATATGTTGGCCCAGCTTCCCTGATTCTTCAGCTTGTACACCGTGCGATAACACTCGATC
AATCTATGCGGTATAATATGTTGGCAAAATTAACTTCTCTGGTGTGTGGAGATGTACACCGTGCGATAACACTCGAT
CAATCTATGCGGTATAATATGTTGGCCCTAACCTATCATCCATCCTTATCTCTTGTACACCGTGCGATAACACTCGA
TCAATCTATGCGGTATAATATGTTGGCTGCACCCAACATTCTAACAAAAGGCTGTACACCGTGCGATAACACTCGAT
CAATCTATGCGGTATAATATGTTGGCCAATAGGTTTTTAAGGAACAGGTGGTGTACACCGTGCGATAACACTCGATC
AATCTATGCGGTATAATATGTTGGCGGGTGATTCCCATTGGCCTGTACACCGTGCGATAACACTCGATCAATCTATG
CGGTATAATATGTTGGCGAAGTAGCTGCTGAGTGATTTGTCTGTACACCGTGCGATAACACTCGATCAATCTATGCG
GTATAATATGTTGGCCCTCTCCACCCTATAGACCCTGTACACCGTGCGATAACACTCGATCAATCTATGCGGTATAA
TATGTTGGCCTAATTAAAGTGGTGTCCCAGATAATCTGTACACCGTGCGATAACACTCGATCAATCTATGCGGTATA
ATATGTTGGCCTCTGACCCATCTAACGCCTATCTGTACACCGTGCGATAACACTCGATCAATCTATGCGGTATAATA
TGTTGGCTGAGTAGCTGGGACTACAGGCATGTACACCGTGCGATAACACTCGATCAATCTATGCGGTATAATATGTT
GGCTGACGCGGTCTCCGCGGTGTACACCGTGCGATAACACTCGATCAATCTATGCGGTATAATATGTTGGCCTTTTT
ATTAAATGCTTTCCATGTATCAAGTTCTGTACACCGTGCGATAACACTCGATCAATCTATGCGGTATAATATGTTGG
CCTTTCCCAATTCTCCTTCAGTCCTGTACACCGTGCGATAACACTCGATCAATCTATGCGGTATAATATGTTGGCGA
TATCAGGGAAGATGAAAAAAGAGACTGTACACCGTGCGATAACACTCGATCAATCTATGCGGTATAATATGTTGGCG
TCCCAGAGGCCCTTGTCAGTGTACACCGTGCGATAACACTCGATCAATCTATGCGGTATAATATGTTGGCCCTGGGC
TCTGTAAAGAATAGTGTACACGAACCGATTGGTGCAGTCTTC
Number of TGTACAs found: 23
No Internal TGTACAs Exist-just Adaptors
Polynucleotide 18
5 primer biotinylation-
ACACCGTGCGATAACACTCGATCAATCTACTTTGAATCCAAGCATTGTTGGGTGACAGAGCAAGACCCTGTACACCG
TGCGATAACACTCGATCAATCTACTTTGAATCCAAGCATTGTGATCCAAGTTGACTTGGCTGAGATGTACACCGTGC
GATAACACTCGATCAATCTACTTTGAATCCAAGCATTGTGCGTTTGTGTGTGCATCTGTAAGCATGTACACCGTGCG
ATAACACTCGATCAATCTACTTTGAATCCAAGCATTGTGTGCACACTTGGACAGCATTTCCTGTACACCGTGCGATA
ACACTCGATCAATCTACTTTGAATCCAAGCATTGTGAGGAAGGGCTGTGTTTCAGGGCTGTACACCGTGCGATAACA
CTCGATCAATCTACTTTGAATCCAAGCATTGTCGCTTTTCTGGCCAGAAACCTCTGTACACCGTGCGATAACACTCG
ATCAATCTACTTTGAATCCAAGCATTGTCCAGCTTCCCTGATTCTTCAGCTTGTACACCGTGCGATAACACTCGATC
AATCTACTTTGAATCCAAGCATTGTAAAATTAACTTCTCTGGTGTGTGGAGATGTACACCGTGCGATAACACTCGAT
CAATCTACTTTGAATCCAAGCATTGTCCTAACCTATCATCCATCCTTATCTCTTGTACACCGTGCGATAACACTCGA
TCAATCTACTTTGAATCCAAGCATTGTTGCACCCAACATTCTAACAAAAGGCTGTACACCGTGCGATAACACTCGAT
CAATCTACTTTGAATCCAAGCATTGTCAATAGGTTTTTAAGGAACAGGTGGTGTACACCGTGCGATAACACTCGATC
AATCTACTTTGAATCCAAGCATTGTGGGTGATTCCCATTGGCCTGTACACCGTGCGATAACACTCGATCAATCTACT
TTGAATCCAAGCATTGTGAAGTAGCTGCTGAGTGATTTGTCTGTACACCGTGCGATAACACTCGATCAATCTACTTT
GAATCCAAGCATTGTCCTCTCCACCCTATAGACCCTGTACACCGTGCGATAACACTCGATCAATCTACTTTGAATCC
AAGCATTGTCTAATTAAAGTGGTGTCCCAGATAATCTGTACACCGTGCGATAACACTCGATCAATCTACTTTGAATC
CAAGCATTGTCTCTGACCCATCTAACGCCTATCTGTACACCGTGCGATAACACTCGATCAATCTACTTTGAATCCAA
GCATTGTTGAGTAGCTGGGACTACAGGCATGTACACCGTGCGATAACACTCGATCAATCTACTTTGAATCCAAGCAT
TGTTGACGCGGTCTCCGCGGTGTACACCGTGCGATAACACTCGATCAATCTACTTTGAATCCAAGCATTGTCTTTTT
ATTAAATGCTTTCCATGTATCAAGTTCTGTACACCGTGCGATAACACTCGATCAATCTACTTTGAATCCAAGCATTG
TCTTTCCCAATTCTCCTTCAGTCCTGTACACCGTGCGATAACACTCGATCAATCTACTTTGAATCCAAGCATTGTGA
TATCAGGGAAGATGAAAAAAGAGACTGTACACCGTGCGATAACACTCGATCAATCTACTTTGAATCCAAGCATTGTG
TCCCAGAGGCCCTTGTCAGTGTACACCGTGCGATAACACTCGATCAATCTACTTTGAATCCAAGCATTGTCCTGGGC
TCTGTAAAGAATAGTGTACACGAACCGATTGGTGCAGTCTTC
Number of TGTACAs found: 23
No Internal TGTACAs Exist-just Adaptors
Polynucleotide 19
5 primer biotinylation-
ACACCGTGCGATAACACTCGATCAATCTCCAGGAGTGCGGGCCCACCATGGGTGACAGAGCAAGACCCTGTACACCG
TGCGATAACACTCGATCAATCTCCAGGAGTGCGGGCCCACCAGATCCAAGTTGACTTGGCTGAGATGTACACCGTGC
GATAACACTCGATCAATCTCCAGGAGTGCGGGCCCACCAGCGTTTGTGTGTGCATCTGTAAGCATGTACACCGTGCG
ATAACACTCGATCAATCTCCAGGAGTGCGGGCCCACCAGTGCACACTTGGACAGCATTTCCTGTACACCGTGCGATA
ACACTCGATCAATCTCCAGGAGTGCGGGCCCACCAGAGGAAGGGCTGTGTTTCAGGGCTGTACACCGTGCGATAACA
CTCGATCAATCTCCAGGAGTGCGGGCCCACCACGCTTTTCTGGCCAGAAACCTCTGTACACCGTGCGATAACACTCG
ATCAATCTCCAGGAGTGCGGGCCCACCACCAGCTTCCCTGATTCTTCAGCTTGTACACCGTGCGATAACACTCGATC
AATCTCCAGGAGTGCGGGCCCACCAAAAATTAACTTCTCTGGTGTGTGGAGATGTACACCGTGCGATAACACTCGAT
CAATCTCCAGGAGTGCGGGCCCACCACCTAACCTATCATCCATCCTTATCTCTTGTACACCGTGCGATAACACTCGA
TCAATCTCCAGGAGTGCGGGCCCACCATGCACCCAACATTCTAACAAAAGGCTGTACACCGTGCGATAACACTCGAT
CAATCTCCAGGAGTGCGGGCCCACCACAATAGGTTTTTAAGGAACAGGTGGTGTACACCGTGCGATAACACTCGATC
AATCTCCAGGAGTGCGGGCCCACCAGGGTGATTCCCATTGGCCTGTACACCGTGCGATAACACTCGATCAATCTCCA
GGAGTGCGGGCCCACCAGAAGTAGCTGCTGAGTGATTTGTCTGTACACCGTGCGATAACACTCGATCAATCTCCAGG
AGTGCGGGCCCACCACCTCTCCACCCTATAGACCCTGTACACCGTGCGATAACACTCGATCAATCTCCAGGAGTGCG
GGCCCACCACTAATTAAAGTGGTGTCCCAGATAATCTGTACACCGTGCGATAACACTCGATCAATCTCCAGGAGTGC
GGGCCCACCACTCTGACCCATCTAACGCCTATCTGTACACCGTGCGATAACACTCGATCAATCTCCAGGAGTGCGGG
CCCACCATGAGTAGCTGGGACTACAGGCATGTACACCGTGCGATAACACTCGATCAATCTCCAGGAGTGCGGGCCCA
CCATGACGCGGTCTCCGCGGTGTACACCGTGCGATAACACTCGATCAATCTCCAGGAGTGCGGGCCCACCACTTTTT
ATTAAATGCTTTCCATGTATCAAGTTCTGTACACCGTGCGATAACACTCGATCAATCTCCAGGAGTGCGGGCCCACC
ACTTTCCCAATTCTCCTTCAGTCCTGTACACCGTGCGATAACACTCGATCAATCTCCAGGAGTGCGGGCCCACCAGA
TATCAGGGAAGATGAAAAAAGAGACTGTACACCGTGCGATAACACTCGATCAATCTCCAGGAGTGCGGGCCCACCAG
TCCCAGAGGCCCTTGTCAGTGTACACCGTGCGATAACACTCGATCAATCTCCAGGAGTGCGGGCCCACCACCTGGGC
TCTGTAAAGAATAGTGTACACGAACCGATTGGTGCAGTCTTC
Number of TGTACAs found: 23

What is claimed is:

1. A method comprising:
obtaining a bead library which includes a bead coupled to a plurality of oligonucleotides, the plurality of oligonucleotides comprising a plurality of first primers of a set of primer pairs for a targeted set of loci, each oligonucleotide further including a first barcode sequence that is common to the plurality of oligonucleotides on the bead;
binding a cell or nucleus of a sample to a polynucleotide concatenate via a binding agent, the polynucleotide concatenate comprising repeating units of univ-bc-gene sequences, represented as
univ-bc-gene1, univ-bc-gene2, . . . , to univ-bc-geneN,
wherein the univ represents a universal sequence, the be represents a second barcode sequence that is common to the repeating units of univ-bc-gene sequences, and the gene1, gene2, . . . , to geneN comprise a respective plurality of second primers of the set of primer pairs for the targeted set of loci, and wherein N represents the number of loci in the targeted set of loci;
wherein the univ and the gene are selected at each gene-univ junction of the univ-bc-gene sequences so as to create a restriction enzyme binding site at each gene-univ junction; and
encapsulating, into a microvesicle, the bead coupled to the plurality of oligonucleotides and the cell or nucleus of the sample that is bound to the polynucleotide concatenate.

2. The method of claim 1, further comprising:
adding restriction endonuclease to the microvesicle for digestion, for separating the univ-bc-gene sequences from the polynucleotide concatenate into a plurality of individual univ-bc-gene units;
causing a membrane of the cell or nucleus to be digested in the microvesicle, for primer access to a genome of the cell or nucleus;
causing the bead to be degraded or digested in the microvesicle, for releasing the plurality of oligonucleotides from the bead; and
performing a polymerase chain reaction (PCR) process for amplifying regions of the genome based on the primer pairs for the targeted set of loci, which generates a plurality of amplicons each of which incorporates the first barcode sequence and the second barcode sequence,
wherein, for each amplicon, the first barcode sequence uniquely identifies the cell or the nucleus and the second barcode sequence uniquely identifies the sample.

3. The method of claim 1, wherein binding the cell or the nucleus of the sample to the polynucleotide concatenate is performed by:
placing the polynucleotide concatenate into a well of a microtiter plate;
placing the binding agent into the well of the microtiter plate, such that the binding agent binds to an end of the polynucleotide concatenate; and
placing the sample of the cell or the nucleus into the well of the microtiter plate, so that the binding agent binds to the membrane of the cell or the nucleus of the sample, thereby binding the cell or the nucleus of the sample to polynucleotide concatenate.

4. The method of claim 3, wherein the polynucleotide concatenate comprises a single-stranded polynucleotide concatenate, the method further comprising:
converting the single-stranded polynucleotide concatenate into a double-stranded polynucleotide concatenate.

5. The method of claim 3, wherein the steps of placing are repeated for each one of a plurality of samples of cells or nuclei that are placed into different wells of the microtiter plate, one sample per well, for binding the cells or the nuclei of each sample to a different polynucleotide concatenate, the method further comprising:
pooling together the different samples of the different cells or the nuclei; and
performing microvesicle encapsulation after pooling together the different samples of the cells or the nuclei.

6. The method of claim 3, wherein:
the binding agent is for binding to different types of cells of the sample for targeting a fraction of the cells of the sample, or
the binding agent comprises an antibody which binds to an epitope of the membrane of the cell or nucleus of the sample, or
the binding agent comprises an antibody which binds to an epitope that is differentially expressed amongst cells of the sample for targeting a fraction of the cells of the sample, or
the binding agent comprises an antibody which binds to an epitope of the membrane of the cell or nucleus of the sample, the polynucleotide concatenate is provided with a moiety on its end, the moiety comprises biotin which binds to streptavidin, and the antibody comprises a streptavidin-conjugated antibody, or
the bead library comprises a vendor bead library, or
wherein the first primers of the set of primer pairs comprise 3′ primers and the second primers of the set of primer pairs comprise 5′ primers, or
wherein the first primers of the set the primer pairs comprise 5′ primers and the second primers of the primer pairs comprise 3′ primers, or
the targeted set of loci comprise Combined DNA Index System (CODIS) Short Tandem Repeats (STRs).

7. The method of claim 1 which is a Placeholder claim, further comprising:
obtaining a bead library which includes a bead coupled to a plurality of oligonucleotides, the plurality of oligonucleotides comprising a plurality of first primers of a set of primer pairs for a targeted set of loci, each oligonucleotide further including a first barcode sequence that is common to the plurality of oligonucleotides on the bead but different from bead to bead;
generating a plurality of polynucleotide concatenates based on at least the bead library, each polynucleotide concatenate comprising repeating units of univ-bc-gene sequences, represented as
univ-bc-gene1, univ-bc-gene2, . . . , to univ-bc-geneN,
wherein the univ represents a universal sequence, the be represents a second barcode sequence that is common to the repeating units of univ-bc-gene sequences in the polynucleotide concatenate but different from the other polynucleotide concatenates, and the gene1, gene2, . . . , to geneN comprise a respective plurality of second primers of the set of primer pairs for the targeted set of loci, and wherein N represents the number of loci in the targeted set of loci;
wherein the univ and the gene are selected at each gene-univ junction of the univ-bc-gene sequences of the plurality of polynucleotide concatenates so as to create a restriction enzyme binding site at each gene-univ junction;
for each one of the plurality of polynucleotide concatenates, placing the polynucleotide concatenate into a respective unique one of a plurality of wells of a microtiter plate, and converting the polynucleotide concatenate into a double-stranded polynucleotide concatenate, thereby producing a plurality of double-stranded polynucleotide concatenates;
placing a binding agent into the plurality of wells, such that the binding agent binds to ends of the plurality of double-stranded polynucleotide concatenates;
for each one of a plurality of samples of cells or nuclei, placing the sample into the respective unique one of the plurality of wells, so that the binding agent binds to a membrane of the cells or the nuclei of the sample, thereby binding the cell or the nucleus of the sample to the double-stranded polynucleotide concatenate, for thereby producing the plurality of samples of cells or nuclei that are respectively bound to the plurality of double-stranded polynucleotide concatenates; and
pooling together the plurality of samples of cells or nuclei that are respectively bound to the plurality of double-stranded polynucleotide concatenates.

8. The method of claim 7, which is also a Placeholder claim, wherein for each one of a plurality of different beads associated with the bead library:
encapsulating, into a microvesicle, the bead coupled to the plurality of oligonucleotides and the cell or nucleus that is bound to the polynucleotide concatenate;
adding restriction endonuclease to the microvesicle for digestion, for separating the univ-bc-gene sequences from the polynucleotide concatenate into a plurality of individual univ-bc-gene units;
causing a membrane of the cell or nucleus to be digested in the microvesicle, for primer access to a genome of the cell or nucleus;
causing the bead to be degraded or digested in the microvesicle, for releasing the plurality of oligonucleotides from the bead; and
performing a polymerase chain reaction (PCR) process for amplifying regions of the genome based on the primer pairs for the targeted set of loci, which generates a plurality of amplicons each of which incorporates the first barcode sequence and the second barcode sequence,
wherein, for each amplicon, the first barcode sequence uniquely identifies the cell or nuclei and the second barcode sequence uniquely identifies the sample.

9. A method comprising:
obtaining a bead library which includes a bead coupled to a plurality of oligonucleotides, the plurality of oligonucleotides comprising a plurality of first primers of a set of primer pairs for a targeted set of loci, each first primer including a first barcode sequence that is common to the plurality of first primers on the bead;
obtaining a plurality of second primers of the set of primer pairs for the targeted set of loci, in bulk, each being represented as univY-gene, wherein the univY represents a universal sequence Y and each gene represents one of the plurality of second primers;
binding a cell or nucleus of a sample to a polynucleotide sequence via a binding agent, the polynucleotide sequence comprising a univX-bc-univY sequence, wherein the univX represents a universal sequence X, the be represents a second barcode sequence, the univY represents the universal sequence Y;
encapsulating, into a microvesicle, the bead coupled to the plurality of oligonucleotides comprising the plurality of first primers of the set of primer pairs, the plurality of second primers of the set of primer pairs, and the cell or the nucleus of the sample that is bound to the polynucleotide sequence.

10. The method of claim 9, further comprising:
causing a membrane of the cell or nucleus to be digested in the microvesicle, for primer access to a genome of the cell or nucleus;
causing the bead to be degraded or digested in the microvesicle, for releasing the plurality of oligonucleotides from the bead; and
performing a polymerase chain reaction (PCR) process for amplifying regions of the genome based on the primer pairs for the targeted set of loci, which generates a plurality of amplicons each of which incorporates the first barcode sequence and the second barcode sequence,
wherein, for each one of at least some of the amplicons, the first barcode sequence uniquely identifies the cell or the nucleus and the second barcode sequence uniquely identifies the sample.

11. The method of claim 9, wherein binding the cell or the nucleus of the sample to the polynucleotide concatenate is performed by:
placing the polynucleotide sequence into a well of a microtiter plate;
placing the binding agent into the well of the microtiter plate, such that the binding agent binds to an end of the polynucleotide sequence; and
placing the sample of the cell or the nucleus into the well of the microtiter plate, so that the binding agent binds to the membrane of the cell or the nucleus of the sample, thereby binding the cell or the nucleus of the sample to polynucleotide sequence.

12. The method of claim 11, wherein the polynucleotide concatenate comprises a single-stranded polynucleotide concatenate, the method further comprising:
converting the single-stranded polynucleotide concatenate into a double-stranded polynucleotide concatenate.

13. The method of claim 11, wherein the steps of placing are repeated for each one of a plurality of samples of cells or nuclei that are placed into different wells of the microtiter plate, one sample per well, for binding the cells or the nuclei of each sample to a different polynucleotide concatenate, the method further comprising:
pooling together the different samples of the different cells or the nuclei; and
performing microvesicle encapsulation after pooling together the different samples of the cells or the nuclei.

14. The method of claim 9, wherein:
the binding agent is for binding to different types of cells of the sample for targeting a fraction of the cells of the sample, or
the binding agent comprises an antibody which binds to an epitope of the membrane of the cell or nucleus of the sample, or
the binding agent comprises an antibody which binds to an epitope that is differentially expressed amongst cells of the sample for targeting a fraction of the cells of the sample, or
the binding agent comprises an antibody which binds to an epitope of the membrane of the cell or nucleus of the sample, the polynucleotide sequence is provided with a moiety on its end, the moiety comprises biotin which binds to streptavidin, and the antibody comprises a streptavidin-conjugated antibody, or
the bead library comprises a vendor bead library, or
wherein the first primers of the set the primer pairs comprise 5′ primers and the second primers of the primer pairs comprise 3′ primers, or
the targeted set of loci comprise Combined DNA Index System (CODIS) Short Tandem Repeats (STRs).

15. A process for single cell next generation sequencing with use of a microfluidic device configured for enabling a generation of emulsions of solution within an immiscible carrier fluid, wherein the generation of the emulsions include single cells with a first set of reagents and/or enzymes desired for carrying out a set of molecular biology reactions, further comprising:
transitioning the single cells into a first incubation chamber within which a first set of molecular biology reactions take place;
merging the emulsions with a second set of reagents and/or enzymes;
transitioning to a second incubation chamber within which a second set of molecular biology reactions take place; and
merging the emulsions with a third set of reagents and/or enzymes, for creating the emulsions for polymerase chain reaction in preparation for sequencing.

16. The process of claim 15, wherein the solution is aqueous or non-aqueous, and wherein the multi-step process is for enabling multiplexed single cell next generation sequencing through tagging of the cells within a collection of samples.

17. The process of claim 15, for use with a simple set of antibody-bound or bead-bound barcode tags or types, and/or bead libraries, for enabling the tagging of cells within microreactors or vessels such that only a fraction of the single cells of the sample are tagged, which constitutes a skimming of cellular diversity from the sample.

18. The process of claim 15, wherein the first set of molecular biology reactions is restriction endonuclease digestion and/or linear amplification using a deoxyribonucleic acid (DNA) polymerase.

19. The process of claim 15, wherein the second set of molecular biology reactions comprise proteinase digestion.

20. The process of claim 15, wherein the third set of molecular biology reactions enables geometric deoxyribonucleic acid (DNA) amplification using a DNA polymerase.
